(19) United States
(12) Patent Application Publication
(10) Pub. No.: US 2002/0146750 A1
(43) Pub. Date: Oct. 10, 2002

(54) Mucin-1 specific binding members and methods of use thereof

(76) Inventors: Hendricus R.J.M. Hoogenboom, Hertogsingel (NL); Maria P.G. Henderikx, Wijngaardstraat (BE)

Correspondence Address: YANKWICH & ASSOCIATES, 130 BISHOP ALLEN DRIVE, CAMBRIDGE, MA, 02139, US

(21) Appl. No.: 09/822,698

(22) Filed: Mar. 30, 2001

Related U.S. Application Data
(63) Continuation-in-part of application No. 09/538,913, filed on Mar. 30, 2000.

(51) Int. Cl. G01N 33/574; C12P 21/02; A61K 39/395; C07K 14/52

(52) U.S. Cl. 435/7.23; 424/155.1; 435/69.5; 530/351; 424/85.1

(57) ABSTRACT

MUC1-specific binding members for cancer-associated MUC1 protein comprise a MUC1 binding domain, or portion thereof, for binding to an epitope of the protein core of MUC1. The MUC1-specific binding members comprise various antibody molecules and fragments thereof, including Fab antibodies; scFv antibodies; double scFv antibodies; diabodies; recombinant, full-length immunoglobulins; and immunocytokine fusion proteins; that are used in methods of diagnosing and treating cancer in various tissues, including breast, ovary, bladder, and lung, and in methods of purifying or isolating MUC1 protein. Polynucleotide molecules encoding MUC1-specific binding members, or portions thereof, are also described.

### FIELD OF THE INVENTION

This invention is generally in the field of the detection and treatment of cancer. In particular, the invention describes molecules that specifically bind to an epitope of the protein core of tumor-associated antigen mucin-1 (MUC-1), which is overexpressed and underglycosylated in human cancers of diverse origins, including breast, ovary, bladder, and lung tissues.

### BACKGROUND OF THE INVENTION

An increasing amount of evidence indicating that cytotoxic T cells, which recognize tumor associated/specific antigens (“TAA”), can selectively kill tumor cells, makes active immunotherapy an attractive option for therapy of cancer (reviewed by Boon et al., Immunol. Today, 18: 267-8 (1997)). The tumor associated glycoprotein mucin-1 (“MUC1”, “MUC-1”), also known as polymorphic epithelial mucin (“PEM”), is one of the most intensively studied targets because, in contrast with normal tissues, it is abundantly present in a non-polar fashion in adenocarcinoma (Burchell et al., Cancer Res.,47: 5476-5482 (1987)). The protein core consists of a high and variable number of tandem repeats (“VNTR”) of 20 amino acids (Gendler et al., J. Biol. Chem. Sep., 263: 12820-12823 (1988)). The tandem repeats are exposed as new peptide epitopes of MUC1 in adenocarcinoma because of their reduced glycosylation compared to MUC1 on normal tissues (Burchell et al., Cancer Res., 47: 5476-5482 (1987)). Murine monoclonal antibodies (“MAb”) against MUC1 have successfully been used to target adenocarcinoma, supporting the potency of MUC1 as a tumor target (Granowska et al., Eur J Nucl Med., 20: 483-489 (1993), Perkins et al., Nucl. Med. Commun., 14: 578-586 (1993), Maraveyas et al., Cancer Res., 55: 1060-1069 (1995), Mariani et al., Cancer Res., 55: 5911s-5915s (1995), Kramer et al., J. Nucl. Med., 34: 1067-74 (1993)).

A cellular cytotoxic response towards MUC1 has been demonstrated in breast cancer and ovarian cancer patients (Ioannides et al., J. Immunol., 151: 3693-703 (1993), Jerome et al., Cancer Res., 51: 2908-16 (1991), Plunkett et al., Cancer Treat Rev., 24: 55-67 (1998)). This response has been associated with a better protection against breast cancer (Jerome et al., Cancer Immunol Immunother., 43: 355-60 (1997)). Active immunotherapy related to MUC1 (reviewed in Plunkett et al., Cancer Treat Rev., 24: 55-67 (1998) and Miles et al., Pharmacol. Ther., 82: 97-106 (1999)) has been studied with variable success in humans and has mainly involved active immunization with non-glycosylated MUC1 peptides containing a VNTR as a source of epitopes that become exposed when MUC1 is expressed in an underglycosylated form by cancer cells. Immunization in humans with (MUC1)₅+Bacillus Calmette-Guérin (BCG) (Goydos et al., J. Surg. Res.,63: 298-304 (1996)) or in animal models with MUC1 presenting dendritic cells (e.g., in mice (Gong et al., Proc. Natl. Acad. Sci. USA., 95: 6279-83 (1998)) or in chimpanzees (Pecher et al., Proc. Natl. Acad. Sci. USA, 93: 1699-704 (1996)) showed, respectively, that it is possible to restore T cell function and to increase the cytotoxic T cell precursor frequency to MUC1. In spite of these reports, and in contrast to results obtained in mice, a poor cytotoxic T cell response and high antibody titers were observed by immunization with MUC1-mannan fusion proteins in humans (Karanikas et al., J. Clin. Invest., 100: 2783-92 (1997)). The B cell response is thought to be related to the presence in humans of natural anti-α-galactosyl (1→3) galactose antibodies which cross-react with MUC1 (Apostolopoulos et al., Nat. Med., 4: 315-20 (1998)). Moreover, amongst its many biological functions, MUC1 inhibits T cell proliferation and it has been postulated that this could be one of the reasons for the presence of anergic tumor infiltrating lymphocytes (TIL) in adenocarcinoma patients (Agrawal et al., Nat. Med., 4: 43-9 (1998), Agrawal et al., Mol. Med Today, 4: 397-403 (1998)). This immunosuppressive effect or anergy may be due either to the direct interaction of soluble or surface bound MUC1 expressed by tumor cells with multiple T cell-receptor molecules (Plunkett et al., Cancer Treat. Rev., 24: 55-67 (1998), Agrawal et al., Nat. Med., 4: 43-9 (1998)), or by the interaction by other, MUC1-associated components, which are not yet identified (Paul et al., Cancer Immunol. Immunother., 48: 22-8 (1999)). Such anergy can be reversed by IL-2 (Agrawal et al., Nat. Med., 4: 43-9 (1998)), and it has been proposed that active immunization with a MUC1 peptide (without any repeats) together with IL-2 administration would be able to stimulate MUC1-specific cytotoxic T lymphocytes (CTLs) (Agrawal et al., Mol. Med. Today, 4: 397-403 (1998)). However, systemic IL-2 administration is known to cause an undesirable nonspecific activation of T cells, and is also associated with dose-dependent toxicity, whose symptoms are known to include malaise, nausea, multi organ failure, shock, and even death (Rosenberg et al., Ann. Surg., 210: 474-84; see, discussion 484-5 (1989)).

It has been demonstrated that IL-2 targeting by immunocytokines (i.e., antibody-cytokine fusion proteins) efficiently impairs growth of other tumor cells due to the induction of CD8⁺ T cell and NK-cell mediated anti-tumor responses (reviewed in Reisfeld et al., J. Clin. Lab. Anal., 10: 160-6 (1996) and Melani et al., Cancer Res., 58: 4146-54 (1998)). In contrast to active therapy using defined TAA-derived molecules, such hybrid fusion proteins may not only stimulate T cells specific for one TAA but also other specific TIL present in the microenvironment of the tumor (Becker et al., Proc. Natl. Acad. Sci. USA., 93: 7826-31 (1996)). Moreover, tumor specific anergic T cells, which are often present in the carcinomas, could be rescued with the IL-2 part of the molecule (Beverly et al., Int. Immunol., 4: 661-671 (1992)).

### SUMMARY OF THE INVENTION

This invention provides various antibody molecules and derivatives thereof, including immuoglobulin molecules and immunocytokine fusion proteins, which are binding members that specifically bind an epitope of the protein core of mucin-1 (MUC1). Such MUC1-specific binding members may be used in the diagnosis and/or treatment of cancer in various tissues, such as adenocarcinomas present in various tissues, especially breast, ovary, bladder, and lung. Variant forms of the MUC1-specific binding members are also provided which possess an additional feature or moiety, which enables the member to be especially useful in diagnosis, imaging, or treatment of cancers. Variants include fusion proteins that possess additional properties, such as MUC1-specific immunocytokine molecules, which have a MUC1 binding domain and a cytokine domain, which provides an additional therapeutic or prophylactic effect on the development or spread of a cancer.

In one embodiment of the invention, MUC1-specific binding members are provided that contain a MUC1 antigen binding domain (MUC1 binding domain) formed from a Fab antibody light chain variable region (V_{L}) and from an antibody heavy chain variable region (V_{H}), or portions thereof. For example, a MUC1-specific binding member of the invention may comprise a V_{L} amino acid sequence of SEQ ID NO:1, and/or a V_{H} amino acid sequence of SEQ ID NO:3, or portions thereof, especially those portions encoding complementarity determining regions (CDRs). Thus, the invention also provides isolated CDRs from MUC1-specific binding domains, such as RSSQSLLHSNGYTYLD (amino acids 24 to 39 of SEQ ID NO:1) for a V_{L} CDR1; SGSHRAS (amino acids 55 to 61 of SEQ ID NO:1), for a V_{L} CDR2; MQGLQSPFT (amino acids 94 to 102 of SEQ ID NO:1) for a V_{L} CDR3; SNAMG (amino acids 31 to 35 of SEQ ID NO:3) for a V_{H} CDR1; GISGSGGSTYYADSVKG (amino acids 50 to 66 of SEQ ID NO:3) for a V_{H} CDR2; HTGGGVWDPIDY (amino acids 99 to 110 of SEQ ID NO:3) for a V_{H} CDR3. One or more of these CDRs may be used to form MUC1 binding domains in a variety of MUC1-specific binding members of the invention.

In another embodiment, the invention provides an isolated MUC1-specific binding member comprising an antigen binding domain, wherein the antigen binding domain comprises an amino acid sequence of the formula:

X₁ X₂ His Thr Gly X₃ Gly Val Trp X₄ Pro X₅ X₆ X₇ (SEQ ID NO:28),

wherein

X₁ is Ala, Ser, Thr, or Val;

X₂ is Lys, Ile Arg, or Gln;

X₃ is Gly, Arg, Val, Glu, Ser, or Ala;

X₄ is Asp or Asn;

X₅ is Ile, Leu, Met, Phe, or Val;

X₆ is Asp, Gly, Lys, Asn, Ala, His, Arg, Ser, Val, or Tyr; and

X₇ is Tyr, His, Lys, Asn, Asp, Ser, Pro.

In a preferred embodiment, the invention provides MUC1-specific binding members comprising an antigen binding domain, wherein the antigen binding domain comprises any of the amino acid sequences listed in Table 9.

In yet another embodiment, the invention provides MUC1-specific binding members comprising a V_{H} region, or CDR thereof, from the DP47 V_{H} germ line and/or a V_{L} region, or CDR thereof, from the DPK15 V_{L} germ line.

In another embodiment, the invention provides MUC1-specific binding members formed by inserting one or more of the CDRs described herein into the framework regions (FRs) of antigen binding domains from other germ lines or from other antibodies.

In still another embodiment, the MUC1-specific binding members of the invention have a MUC1-specific binding domain comprising a V_{L} and/or V_{H} region, or portions thereof, as described above, and is an antibody molecule selected from the group consisting of full-length immunoglobulin molecules (such as, IgG, IgM, IgA, IgE), Fab antibodies, F(ab′)₂ antibodies, diabodies, single chain antibody (scFv) molecules, Fv molecules, double-scFv molecules, domain antibody (dAb) molecules, and immunocytokines. MUC1-specific, full-length immunoglobulin molecules of the invention include recombinant immunoglobulin proteins in which the V_{L} and/or V_{H} region of a MUC1-specific Fab antibody has been genetically engineered into a complete, human immunoglobulin molecule, such as a human antibody of isotype IgG1. The benefits of such a recombinant, full-length, human immunoglobulin with MUC1 binding specificity derived from a Fab antibody include the presence of two contiguous MUC1 binding sites, a decreased immunogenicity to avoid the classic HAMA response in humans, an enhanced half-life in humans, and a significantly enhanced affinity for MUC1 expressed on cancer cells and tissues, particularly ovarian and breast cancer cells and tissues, compared to the single MUC1 binding site of the corresponding Fab antibody. The MUC1-specific immunoglobulins of the invention include isotypic variants and allotypic variants.

Preferred embodiments of MUC1-specific immunoglobulins provided by the invention include immunoglobulin molecules comprising a V_{L} having the amino acid sequence of SEQ ID NO:1 and a V_{H} having the amino acid sequence of SEQ ID NO:3. In another preferred embodiment, the invention provides a recombinant, human immunoglobulin, which comprises a light chain (i.e., V_{L} and C_{L} kappa light chain constant region) having the amino acid sequence of SEQ ID NO:24 and a heavy chain (V_{H} and C_{H} heavy chain constant region for the human gamma-1 isotype) having the amino acid sequence of SEQ ID NO:26.

In another preferred embodiment, a MUC1-specific binding member of the invention is an immunocytokine, which comprises a MUC1-specific binding domain and a cytokine domain, which confers an immunomodulatory activity on the MUC1-specific binding member. Preferred cytokines for use in such MUC1-specific binding members include IL-2, GM-CSF, and TNF, or portions thereof, though others may be used. More preferably, the immunocytokine is a fusion protein comprising a diabody fused to a cytokine, such as the IL-2 cytokine. Most preferably, the immunocytokine is the bivPH1-IL-2 of the invention having the amino acid sequence of SEQ ID NO:5.

In another aspect of the invention, variant forms of MUC1-specific binding members are provided that are linked, preferably covalently, to other molecules, including, but not limited to other proteins, polypeptides, peptides, such as cytokines or enzymes; anti-cancer drugs; fluorescent labels; radioactive compounds, such as magnetic resonance imaging compounds or anti-cancer radioactive compounds; and heavy metals. Such variants are especially well suited for use in the diagnostic, imaging, purification, or therapeutic methods of the invention.

The invention also provides MUC1-specific binding members that are proteins, polypeptides, and peptides that comprise an amino acid sequence that is homologous to any of the amino acid sequences described herein. Such homologous proteins, polypeptides, or peptide molecules bind MUC1 or form part of a MUC1-specific binding domain and comprise an amino acid sequence that is about 70% or more, preferably about 80% or more, or more preferably about 90%, 95%, 97%, or even 99% or more homologous to an amino acid sequence described herein. Even more preferably, such a homologous protein, polypeptide, or peptide of the invention comprises a V_{H} and/or V_{L} region, or CDR thereof, that is about 70% or more, preferably about 80% or more, and more preferably about 90%, 95%, 97%, or 99% or more homologous to the amino acid sequence of SEQ ID NO:1 (for the V_{L} region, and CDRs therein) and/or to the amino acid sequence of SEQ ID NO:3 (for the V_{H} region, and CDRs therein).

In another embodiment, the invention provides MUC1-specific binding members and portions thereof, such as a V_{L} or V_{H} region, or CDR, that comprise an amino acid sequence described herein in which one or more of the amino acids have been conservatively substituted with another amino acid.

The invention also provides methods of diagnosing MUC1-expressing cancer, such as adenocarcinoma, using MUC1-specific binding members and variants thereof. Such diagnostic methods comprise contacting cells, tissues, or a body fluid of an individual with a MUC1-specific binding member and detecting the MUC1-specific binding member bound to MUC1 on the cells or tissues or present in the fluid of the individual. Preferably, the methods of the invention are used to diagnose ovarian, breast, bladder, and lung cancer. Diagnostic methods of the invention include the use of a MUC1 binding member described herein in methods of imaging cells, tissues, and/or organs to detect the presence of a cancer in the cells, tissues, and/or organs.

In another embodiment, the MUC1-specific binding members and variants thereof may be used in methods of purifying cancer-associated MUC1, underglycosylated forms of MUC1, or non-glycosylated MUC1 molecules in a mixture or extract.

In yet another embodiment, MUC1-specific binding members, and variants thereof, may be used in methods for therapeutically or prophylactically treating MUC1-expressing cancer in an individual. The treatment methods of the invention may be in vivo or ex vivo methods. The in vivo methods of treating cancer comprise administering to an individual a MUC1-specific binding member, or variant thereof, described herein. The MUC1-specific binding member, or variant thereof, may be administered by any of a variety of routes including parenterally, such as intravenously or intramuscularly; orally; by inhalation; topically; or by direct injection into or close to a tumor or affected site. Various pharmaceutical compositions comprising a MUC1-specific member may be prepared that are particularly suited for a chosen route of administration. Preferably, the MUC1-specific binding member is administered parentally, and more preferably intravenously. In a preferred method of treatment, the MUC1-specific binding member is an immunocytokine or is an immunoglobulin, which may be linked to an anti-tumor compound. More preferably, the method of treatment comprises administering the immunocytokine bivPH1-IL-2 having the amino acid sequence of SEQ ID NO:5 or the immunoglobulinn comprising light chains having the amino acid sequence of SEQ ID NO:24 and heavy chains having the amino acid sequence of SEQ ID NO:26.

More preferably, the method of treating a cancer using an immunocytokine described herein comprises administering to an individual an unconjugated (free) form of a cytokine before, contemporaneously with, or after administering an immunocytokine described herein.

A preferred method of treating a cancer according to the invention comprises administering to an individual in need of treatment a MUC1-specific immunoglobulin described herein linked (preferably covalently) to an anti-cancer compound, such as a derivative or variant of doxorubicin or a toxin molecule.

In another aspect of the invention, ex vivo methods of cancer treatment comprise extracting cells, tissues, or a body fluid from an individual, contacting the extracted cells, tissues, or body fluid with a MUC1-specific binding member, or variant thereof, as described herein; collecting the cells, tissues, or body fluid depleted or purged of cancer-associated MUC1 and/or MUC1-expressing cancer cells; and then returning the remaining cells, tissues, or body fluid, which do not express or contain cancer-associated MUC1 to the individual.

It is another aspect of the invention to provide polynucleotide molecules encoding the various MUC1-specific binding members, V_{L} region, V_{H} region, CDRs, and framework (FR) regions described herein.

In a preferred embodiment, isolated polynucleotide molecules are provided that encode the V_{L} and/or V_{H} region, or portions thereof, of the binding domain of a MUC1-specific binding member, such as the PH1 Fab antibody described herein.

In another preferred embodiment, the polynucleotide molecules comprise the nucleotide sequence of SEQ ID NO:2 encoding a V_{L} region having the amino acid sequence of SEQ ID NO:1, or portions thereof, and/or the nucleotide sequence of SEQ ID NO:4 encoding a V_{H} region having the amino acid sequence of SEQ ID NO:3, or portions thereof.

In another preferred embodiment, the invention provides polynucleotide molecules comprising nucleotide sequences that encode one or more CDRs from an antibody V_{L} or V_{H} region of the PH1 Fab antibody such as: AGGTCTAGTCAGAGCCTCCTGCATAGTAATGGATACACCTATTTGGAT (nucleotides 70-117 of SEQ ID NO:2), which encodes a V_{L} CDR1; TCGGGTTCTCATCGGGCCTCC (nucleotides 163 to 183 of SEQ ID NO:2), which encodes a V_{L} CDR2; ATGCAGGGTCTACAGAGTCCATTCACT (nucleotides 280-306 of SEQ ID NO:2), which encodes a V_{L} CDR3; AGTAACGCCATGGGC (nucleotides 91 to 105 of SEQ ID NO:4), which encodes a V_{H} CDR1; GGTATTAGTGGTAGTGGTGGCAGCACATACTACGCAGACTCCGTGAAGGGC (nucleotides 148-198 of SEQ ID NO:4), which encodes a V_{H} CDR2; CATACCGGGGGGGGCGTTTGGGACCCCATTGACTAC (nucleotides 295 to 330 of SEQ ID NO:4), which encodes a V_{H} CDR3; and combinations thereof.

The polynucleotide molecules of the invention also include polynucleotide molecules comprising degenerate forms of one or more of the previously mentioned nucleotide sequences, which encode the same protein, polypeptide, or peptide.

In yet another embodiment of the invention, polynucleotide molecules are provided which have a nucleotide sequence that is homologous to any of the nucleotide sequences listed herein. A homologous polynucleotide molecule of this invention comprises a nucleotide sequence that is about 60%, more preferably 70%, even more preferably 80%, and most preferably 90%, 95%, 97%, or even 99% or more, homologous to a nucleotide sequence described herein that encodes a MUC1-specific binding member, a MUC1-specific binding domain, or a portion thereof, such as a CDR or a CDR and selected amino acid residues of an adjacent FR of a MUC1-specific binding domain.

The invention also provides methods of producing MUC1-specific binding members using the polynucleotide molecules described herein. Such polynucleotide molecules may be inserted in any of a variety of prokaryotic or eukaryotic vectors for production of a MUC1-specific binding member in cultures of appropriate prokaryotic or eukaryotic host cells. Such vectors useful in the methods of the invention include plasmids, phage, phagemids, and eukaryotic viral vectors.

In another embodiment of the invention, MUC1-specific binding members of the invention are expressed and displayed on the surface of cells or phage particles. Preferably, MUC1-specific binding members described herein are expressed and displayed on the surface of cells or phage particles using phage, phagemid, or yeast display vectors.

### BRIEF DESCRIPTION OF THE DRAWINGS

FIG. 1 shows diagrams (A-D) of the cloning schedule for the construction of the bivalent diabody bivPH1 and bivPH1-IL-2 immunocytokine. FIG. 1A is a diagram of the starting PH1 Fab gene in the vector plasmid pCES1. FIG. 1B is a diagram of the cloning of the PH1 V_{H} and restriction sites into the plasmid vector pCantab6. FIG. 1C illustrates the insertion of the PH1 V_{L} to retrieve the bivPH1 diabody from the plasmid vector pKaPa1. FIG. 1D diagrams the construction of plasmid pKaPa2 by insertion of the IL-2 coding sequence to retrieve the bivalent immunocytokine bivPH1-IL-2. Abbreviations: pLacZ, the LacZ promoter; rbs, ribosome binding site; S, signal sequence; PH1VH, heavy chain variable region of Fab fragment PH1; PH1VL, light chain variable region of Fab fragment PH1; H, tag encoding 6 histidines; tag, myc-tag sequence; *, stop codon; L1, linker 1 nucleotide sequence encoding 5 amino acid L1 linker peptide; L2, linker 2 nucleotide sequence encoding 9 amino acid L2 linker peptide.

FIG. 2 shows the graphs of the binding characteristics of different antibody formats on BIAcore. Abbreviations: open triangles, scFv 10A; open circles, Fab PH1; open squares, bivalent diabody bivPH1-IL-2. MUC1 80-mer was coupled to a chip at a density of 90 Response Units (RU), binding of the three MUC1 antibodies was measured.

FIGS. 3A and 3B show a comparison of the binding of antibodies scFv 10A, PH1 Fab, bivPH1 diabody, bivPH1-IL-2 immunocytokine to cell lines 3T3, the 3T3 MUC1-transfected cell line ETA, OVCAR-3, T47D and LS174T in flow cytometry. Binding characteristics of the antibodies to the different cell lines are given in overlayed histograms. Binding intensities of the antibodies to the cells were measured by secondary staining with FITC-labeled antibodies, and fluorescence was measured (FL1-H). Number of stained cells were measured (COUNTS). Unbroken line indicates binding of antibody; alternating broken and dotted line indicates negative control (in the case of the 3T3 MUC1-transfected cell line ETA, the negative control was the non-transfected cell line 3T3); and broken line indicates competition for cell binding with MUC1 60-mer.

FIG. 4 shows the results of induction of CTLL-16 proliferation by rIL-2 (open circles) and bivPH1-IL-2 (open squares) by uptake of radioactive ³H-thymine measured in counts per minute (cpm).

FIG. 5 shows the results of stimulation of resting PBL by rIL-2 or bivPH1-IL-2, without or with the addition of MUC1 measured by ³H-thymidine uptake assay. Medium alone (stipled bars); PHA without MUC1 (open bars); PHA with MUC1 (diagonal bars). Uptake of ³H-thymidine was measured in counts per minute (cpm).

FIG. 6 shows the results of the ⁵¹chromium-release assay with antibody coated OVCAR-3 target cells (T) by resting PBL effector cells (E). E:T ratios: 100:1 (stipled bars); 50:1 (white bars); 25:1 (horizontal bars); 12.5:1 (diagonal bars). Percent (%) lysis of the OVCAR-3 target cells was calculated by 100×(cpm test ⁵¹Cr released−cpm minimal ⁵¹Cr released/cpm maximal ⁵¹Cr released−cpm minimal ⁵¹Cr released).

### DETAILED DESCRIPTION

The invention provides MUC1-specific binding members that preferentially bind to the protein core of MUC1. The specific binding members of MUC1 described herein include those binding members that comprise a MUC1 antigen binding domain, which comprises a variable light chain region (V_{L}) having the amino acid sequence of SEQ ID NO:1, or portion thereof, such as one or more of the complementarity deteremining regions (CDRs) of V_{L}, and/or a variable heavy chain region (V_{H}) having the amino acid sequence of SEQ ID NO:3, or portion thereof, such as one or more CDRs of V_{H}, as found in or isolated from a human Fab antibody or monoclonal antibody (MAb). As discussed below, MUC1-specific binding members of the invention may be fusion or recombinant proteins. Such fusion proteins include those that comprise a MUC1-specific binding portion and an immunomodulatory portion, such as a cytokine, such as IL-2, or active fragment thereof. The recombinant proteins of the invention include recombinant, immunoglobulin molecules, in which a MUC1-specific binding domain of a Fab antibody or other binding member has been engineered into an immunoglobulin molecule. Such recombinant immunoglobulins exhibit enhanced affinity and avidity for MUC1 over MUC1-binding members that have a single MUC1 binding site.

The MUC1-specific binding members of the invention may be used to diagnose or treat cancer, such as adenocarcinoma, which may be found in a wide variety of tissues including mammary (e.g., breast cancer), ovary, lung, and bladder and which is characterized by overexpression of a glycoform of MUC1 . MUC1 molecules that are produced by cancer cells and tissues (cancer-associated MUC1) are underglycosylated and, therefore, expose the core protein epitopes that are recognized and bound by the MUC1-specific binding members described herein.

In order that the invention may be more fully understood, the following terms are defined:

“Specific binding member” or “binding member” as used and understood herein, refers to a member of a pair of molecules, which have binding specificity for one another. The members of such a specific binding pair may be naturally derived or wholly or partially synthetically produced. One member of the pair of molecules has an area on its surface, or a cavity, which specifically binds to and is therefore complementary to the three-dimensional geometry and chemistry of the other member of the pair of molecules. Thus, the members of the binding pair have the property of binding specifically to each other. Examples of types of specific binding pairs are antigen-antibody, biotin-streptavidin or avidin, hormone-hormone receptor, receptor-ligand, enzyme-substrate. It is also understood that one member of a specific binding pair may also be a member of other specific binding pairs, for example, as is the case with an antigenic protein and different antibodies, where each antibody binds to a different site (epitope) on the same antigen or to the same site, but with a different or same affinity or avidity. This invention is concerned with antigen-antibody type binding members. More specifically, this invention is concerned with specific binding member pairs consisting of a MUC1-specific binding member molecule, such as an antibody molecule as defined below, which has an antigen binding site formed by a variable light (V_{L}) chain region, or portion thereof, and/or variable heavy (V_{H}) chain region, or portion thereof, from a human Fab antibody and of the other binding member of the pair, which is a protein or polypeptide that comprises a MUC1 VNTR (variable number of tandem repeats) protein core amino acid sequence.

“Antibody” or “antibody molecule”, as used and understood herein, refers to a specific binding member that is a protein molecule or portion thereof or any other molecule, whether produced naturally, synthetically, or semi-synthetically, which possesses an antigenic binding domain formed by an immunoglobulin variable light chain region or domain (V_{L}), or portion thereof, and/or an immunoglobulin variable heavy chain region or domain (V_{H}), or portion thereof. The term also covers any polypeptide or protein molecule that has an antigen binding domain which is identical, or homologous to, an antibody binding domain of an immunoglobulin. Examples of an antibody molecule, as used and understood herein, include any of the well known classes of immunoglobulins (e.g., IgG, IgM, IgA, IgE, IgD) and their isotypes; fragments of immunoglobulins that comprise an antigen binding domain, such as Fab or F(ab′)₂ molecules; single chain antibody (scFv) molecules; double scFv molecules; single domain antibody (dAb) molecules; Fd molecules; and diabody molecules. Diabodies are formed by association of two diabody monomers, which form a dimer that contains two complete antigen binding domains wherein each binding domain is itself formed by the intermolecular association of a region from each of the two monomers (see, e.g., Holliger et al., Proc. Natl. Acad. Sci. USA, 90: 6444-6448 (1993)).

It is possible to take an antibody molecule, such as a Fab antibody or monoclonal antibody (MAb) molecule, and use techniques of recombinant DNA technology available in the art to produce other molecules, which retain the specificity of the original (parent) antibody or a particular region of the original antibody. Such techniques may involve introducing DNA comprising a nucleotide sequence(s), which, for example, encodes the immunoglobulin variable regions of the variable light (V_{L}) and/or variable heavy (V_{H}) immunoglobulin chains of a Fab or other MUC1-specific antibody, or which encodes portions of the V_{L} and/or V_{H}, such as one or more of the complementarity determining regions (CDRs), in frame with another DNA sequence, such as a nucleotide sequence encoding an immunoglobulin constant region or constant region and framework (FR) regions of a different immunoglobulin (see, e.g., EP-A-184187, GB 2188638A, EP-A-239400). For example, new, recombinant MUC1-specific immunoglobulins may be produced by cloning nucleotide sequences encoding V_{L} and V_{H} regions, or portions thereof, from one (parent) MUC1-binding member, into plasmid expression vectors used for expressing the light and heavy chains of an immunoglobulin molecule, such as an IgG. The recombinant plasmids are then transfected into a compatible host cell for expression of the recombinant immunoglobulin, which has the MUC1-binding specificity of the parent molecule. Such recombinant immunoglobulins may also exhibit enhanced avidity for MUC1 compared to the parent molecule, owing to the divalent structure (two identical binding sites) for MUC1 and/or other features (see, e.g., Example 3). A hybridoma or other cell that produces an antibody molecule may also be subjected to genetic mutation or other changes, which may alter the binding specificity or other property of the antibody molecule produced by that cell to form a new MUC1 binding member of this invention.

As antibodies can be modified in a number of ways, the term “antibody” is understood to cover any specific binding member or substance having a binding domain as described herein with the required specificity for the other member, i.e., MUC1. Thus, “antibody” or “antibody molecule” covers antibody fragments, derivatives, functional equivalents and homologues of antibodies, including any polypeptide comprising an immunoglobulin binding domain, whether natural or wholly or partially synthetic. Fusion or chimeric protein molecules comprising an immunoglobulin binding domain or CDRs thereof, or equivalent, fused to another polypeptide, such as a cytokine, another immunoglobulin, enzyme, or protein toxin, are also included. Cloning and expression of some examples of chimeric antibodies are described in EP-A-0120694 and EP-A-0 125023.

Various fragments of a whole immunoglobulin molecule are generally known to be capable of performing the function of binding antigens or of being recombined, for example using recombinant DNA methods, to form binding members with the same specificity as a whole immunoglobulin but having a smaller size. For example, classically a Fab fragment is an antibody that can be generated by papain digestion of an immunoglobulin molecule and has a single antigen binding domain (monovalent) consisting of the V_{L}, V_{H}, the constant domain of the light chain (C_{L}), and the CH1 constant domain of the heavy chain. Fab antibodies can also be produced synthetically or in vivo from cells containing recombinant expression vectors, which encode and express a particular Fab antibody. Fab antibodies of the invention also include those molecules selected from a phage display library of human Fab molecules for the ability to bind a MUC1 epitope (see, e.g., Examples 1 and 2). A F(ab′)₂ fragment is an antibody, which classically has been generated by pepsin digestion of an immunoglobulin molecule to yield two linked Fab fragments and, therefore, two complete antigen binding domains (bivalent), which are capable of binding and cross-linking antigen molecules. An Fd fragment or antibody consists of the V_{H} and CH1 domains of the immunoglobulin heavy chain. Another example of a portion of an immunoglobulin that is capable of binding the same antigen as full-length immunoglobulin is an Fv antibody molecule consists of the V_{L} and V_{H} regions of a single immunoglobulin (and absent constant domains). Another antigen-binding portion of a full-length immunoglobulin is a dAb fragment or antibody, which consists of a V_{H} domain (Ward, et al., Nature, 341: 544-546 (1989)). In addition, an isolated CDR region, either alone or together with one or more other CDRs of an immunoglobulin, may form an antigen binding domain. A single chain Fv (scFv) antibody molecule is a monovalent molecule wherein a V_{H} domain and a V_{L} domain are linked by a peptide linker, which allows the two variable domains to associate intramolecularly to form a complete antigen binding site (see, e.g., Bird et al., Science, 242: 423-426 (1988); Huston et al., Proc. Natl. Acad. Sci. USA, 85: 5879-5883 (1988)). It is also possible to form bispecific scFv dimers, which bind two different epitopes (see, e.g., PCT/US92/09965). Diabodies (discussed in more detail below) may be bivalent or even multivalent or multispecific molecules are also typically constructed by gene fusion in which a DNA molecule encoding one or more V_{L} domains is linked in frame with a DNA molecule encoding one or more V_{H} domains.

Diabodies (or diabody antibodies) are multimers (e.g., dimers, tetramers) of polypeptides, wherein each polypeptide comprises a V_{L} region and V_{H} region of an immunoglobulin antigen binding domain that are linked to one another, e.g., by a relatively short peptide linker, such that the two regions are unable to associate with each other intramolecularly to form an antigen binding site. Complete antigen binding domains are only assembled intermolecularly by the association of the V_{L} domain of one polypeptide (monomer) with the V_{H} domain of another polypeptide (monomer) which occurs when a multimer forms (see, e.g., PCT publication number WO 94/13804; P. Holliger et al., Proc. Natl. Acad. Sci. USA, 90: 6444-6448 (1993)).

Where bispecific antibodies, i.e., antibody molecules having binding domains for two different antigens or epitopes, are to be used, these may be conventional bispecific immunoglobulin antibodies, which can be produced by various techniques, including, for example, by chemical modifications, from hybrid hybridomas, or by recombinant immunoglobulin expression vectors transfected into appropriate host cells, or may be any of the bispecific antibody fragments mentioned above (see, e.g., Holliger and Winter, Current Opinion Biotechnol., 4: 446-449 (1993)). Alternatively, it may be preferable to use scFv dimers or diabodies, rather than whole antibodies. Diabodies and scFv molecules can be constructed using variable domains without an Fc region in order to reduce potential effects of anti-idiotypic reactions. Other forms of bispecific antibodies include the single chain “Janusins” described in Traunecker et al., EMBO J., 10: 3655-3659 (1991).

Bispecific diabodies, as opposed to bispecific whole immunoglobulin molecules, may also be particularly useful because they can be conveniently constructed and expressed in procaryotic cells, such as E. coli. Furthermore, diabodies and many other antibody fragments, as described above, of appropriate binding specificity can be readily selected from libraries using phage display (see, e.g., WO 94/13804 and Examples below). In addition, bispecific diabodies may be constructed by maintaining one domain of the diabody having a specificity that is directed against one antigen, while selecting from a library for a different specificity in the other binding domain.

“Antigen”, as used and understood herein refers to any molecule that can elicit an immune response and/or that can be bound by an antibody. An antigen as used herein is not limited by molecular size and includes any molecule, whether produced naturally, synthetically, or semi-synthetically, which can be bound by an antibody molecule. In addition, it is understood that an antigen molecule has one, several, or many different sites at which an antibody may bind.

“Antigenic determinant” or “epitope” are used synonymously and refer to the specific site on an antigen at which an antibody molecule binds. The antigenic determinant or epitope of an antigen is complementary to the antigen binding domain (see, below) of an antibody. An antigen may have only one or, as is usually the case, several or even many epitopes. Epitopes of a given antigen molecule may be present as multiple copies of structurally identical moieties, as in case of repetitive amino acid sequences in a protein, or distinctly different, in which case each epitope could be bound by a different antibody.

“Antigen binding domain,” as used and understood herein refers to the region of an antibody molecule which specifically binds to and is complementary to a particular site on an antigen, which is a specific binding member or partner to the antibody molecule. An antigen binding domain may be provided by one or more antibody variable regions. The antigen binding domain of an immunoglobulin antibody or fragment thereof, such as a Fab or F(ab′)₂ antibodies, comprises an antibody V_{L} region and an antibody V_{H}, which variable regions consists of complementarity determining regions (CDRs) and framework regions (FRs). CDRs are highly variable regions within the V_{L} and V_{H} regions of an antibody and contain the critical amino acid sequences for the specificity and avidity for binding to a particular site (i.e., an epitope) on an antigen (see, e.g., Fundamental Immunology, 4th ed. (Paul, William E., ed.) (Lippincott-Raven, Pa., 1999), pages 58-60). CDRs are located among framework regions (FRs), which provide a structural context to the variable regions necessary for binding to a specific site on an antigen. Using recombinant DNA techniques, it is possible to construct DNA molecules that code for each variable region or domain (V_{L}, V_{H}), or even portions of a variable region, such as individual CDRs or a CDR and contiguous residues of adjacent FRs, which in turn may be inserted into a gene coding for a different antibody, or other protein to form a recombinant antibody protein that has a new antigen binding domain (see, e.g., Example 3).

“Specific,” as used and understood herein refers to the preference of one member of a specific binding pair to bind with the other member. The term is also applicable where an antigen binding domain is specific for a particular epitope which is carried by a number of antigens, in which case the specific binding member carrying the antigen binding domain will be able to bind to the various antigens carrying that epitope. Likewise the term is applicable where an antigen binding domain is specific for a particular epitope of a binding member and the same antigen binding domain is carried by different types of antibody molecules, e.g., scFv or Fab antibodies, in which case the different types of antibody molecules are able to bind to and are, therefore, understood to be, “specific” for the same epitope. “Functionally equivalent variant” or simply “variant”, unless noted otherwise, as used and understood herein, refers to a molecule (the variant), which although having structural differences from another molecule (the parent), has retained some significant homology or at least some of the biological function of the parent molecule, such as the ability to bind a particular antigenic determinant or epitope of MUC1. Variants may be in the form of fragments, such as Fabs or F(ab′)₂ antibodies, which are fragments of larger immunoglobulin molecules, or mutant antibody protein molecules in which the amino acid sequence of a parent antibody protein has been altered to yield a variant antibody, which retains the specificity of the parent for an epitope, but now has an enhanced (or, for some applications, possibly decreased) avidity for the epitope. For example, a selected antibody can be affinity matured for enhanced affinity for an antigen or epitope according to procedures known to persons skilled in the art and described herein by introducing diversity in a nucleotide sequence of a polynucleotide molecule encoding the parent antibody, or portion thereof, by replacing the V_{H} or V_{L} genes with a repertoire of V_{H} or V_{L} genes or by introducing mutations, and then selecting variants against the desired antigen or epitope by phage display (see, e.g., Example 2, De Haard et al., Adv. Drug Del. Rev., 31: 5-31 (1998); Hoogenboom et al., Trends in Biotech., 15: 62-70 (1997)). The variants can then be screened for enhanced affinity.

Variant mutant proteins may be produced synthetically or biologically using recombinant DNA techniques in which case the variant is the expressed product (mutant protein) of a mutated gene. A variant protein may also be formed by linking, preferably covalently, the parent molecule to another protein or molecule, such as a cytokine, a toxin, an epitope tag (such as hemagglutinin or myc epitope tags), a fluorescein dye, streptavidin, biotin, enzyme, or radioactive compound, such ¹²⁵I or ^{99m}Tc, and the like. As with mutant proteins, such variant forms may be produced as determined by the skilled practitioner, at the nucleotide or protein level. For example, the encoded polypeptide may be a Fab fragment which is then linked to an Fc tail from another source, such fusion proteins are usually made using recombinant DNA methods. Alternatively, a marker or label compound, such as an enzyme, fluorescent molecule, radioactive compound, or biotin, are usually linked directly to the parent molecule.

“Homologues” of the MUC1-binding members described herein may be formed by substitution, addition, or deletion of one or more amino acids employing methods well known in the art and for particular purposes known in the art. Such “homologous” proteins, polypeptides, or peptides will be understood to fall within the scope of the present invention so long as the substitution, addition, or deletion of amino acids does not eliminate its ability to bind MUC1 or to form part of a MUC1 binding domain. The term “homologous”, as used herein, refers to the degree of sequence similarity between two polymers (i.e., polypeptide molecules or nucleic acid molecules). When the same nucleotide or amino acid residue occupies a sequence position in the two polymers under comparison, then the polymers are homologous at that position. For example, if the amino acid residues at 60 of 100 amino acid positions in two polypeptide sequences match or “are homologous”, then the two sequences are 60% homologous. The homology percentage figures referred to herein reflect the maximal homology possible between the two polymers, i.e., the percent homology when the two polymers are so aligned as to have the greatest number of matched (homologous) positions. Various computer programs are available for aligning two polymers and also for calculating the percent homology between the two polymers. For example, alignment and/or percent homology calculations between two polymers of interest are routinely performed using the BLAST sequence bank computer program (see, e.g., http://www.ncbi.nlm.nih.gov/blast/) or the MCVECTOR® computer program. For germ line homology studies, Vbase (see, e.g., http://www.mrc-cpe.cam.ac.uk/imt-doc/) performs alignments between new and known germ line sequences in order to determine the source of individual V_{L} or V_{H} regions of an antibody molecule. Protein, polypeptide, and peptide homologues within the scope of the present invention will be about 70%, preferably about 80%, and more preferably about 90% or more (including about 95%, about 97%, or even about 99% or more) homologous to a MUC1-binding member, a MUC1 binding domain, or portion thereof, including a CDR or a CDR and selected contiguous framework (FR) residues, as disclosed herein. Polynucleotide homologues within the scope of the present invention will be about 60%, preferably about 70%, more preferably about 80%, and even more preferably about 90% or more (including about 95%, about 97%, or even about 99% or more) homologous to the nucleotide sequences described herein that encode a MUC1-specific binding member, a MUC1 binding domain, or portion thereof (such as V_{L}, V_{H}, CDR), as disclosed herein.

The amino acid sequences of the proteins, polypeptides, and peptides described herein are recited using either the conventional one letter or three letter abbreviations for amino acids known in the art.

Anti-MUC1 PH1 Fab Antibody

The origin of the MUC1 binding domain of all of the MUC1-specific binding members of the invention is an anti-MUC1 human Fab fragment (Fab antibody), designated PH1, which was obtained by screening a naive (non-immunized) phage display library containing 3.7×10¹⁰ different Fab fragments (see, Examples below). The phage displaying the PH1 Fab fragment was identified and isolated by selection and screening for the ability to bind a VNTR sequence of the MUC1 core protein and for binding to MUC1-expressing cells. The genes encoding the V_{H} and V_{L} regions of PH1 encoded on a phagemid were isolated and sequenced. The PH1 V_{L} region is encoded by the nucleotide sequence of SEQ ID NO:2 and has the amino acid sequence of SEQ ID NO:1. The PH1 V_{H} region is encoded by the nucleotide sequence of SEQ ID NO:4 and has the amino acid sequence of SEQ ID NO:3. Each variable region of the PH1 Fab antibody contains both structural framework (FR) sequences and the highly variable complementarity-determining regions (CDRs), which confer the specificity and avidity of the antigen-binding domain for the epitope of MUC1.

For the V_{L} region of the PH1 Fab molecule, CDR1 is encoded by the nucleotide sequence and reading frame AGG TCT AGT CAG AGC CTC CTG CAT AGT AAT GGA TAC ACC TAT TTG GAT (nucleotides 70 to 117 of SEQ ID NO:2) and has the amino acid sequence of RSSQSLLHSNGYTYLD (amino acids 24 to 39 of SEQ ID NO:1); CDR2 is encoded by the nucleotide sequence and reading frame TCG GGT TCT CAT CGG GCC TCC (163 to 183 of SEQ ID NO:2) and has the amino acid sequence of SGSHRAS (amino acids 55 to 61 of SEQ ID NO:1); and CDR3 is encoded by the nucleotide sequence and reading frame ATG CAG GGT CTA CAG AGT CCA TTC ACT (nucleotides 280 to 306 of SEQ ID NO:2) and has the amino acid sequence of MQGLQSPFT (amino acids 94 to 102 of SEQ ID NO:1). FR1 of the V_{L} region of PH1 is encoded by the nucleotide sequence and reading frame GAA ATT GTG CTG ACT CAG TCT CCA CTC TCC CTG CCC GTC ACC CCT GGA GAG CCG GCC TCC ATC TCC TGC (nucleotides 1 to 69 of SEQ ID NO:2) and has the amino acid sequence of EIVLTQSPLSLPVTPGEPASISC (amino acids 1 to 23 of SEQ ID NO:1); FR2 of the V_{L} region of PH1 is encoded by the nucleotide sequence and reading frame TGG TAC CTG CAG AAG CCA GGG CAG TCT CCA CAG CTC CTG ATC TAT (nucleotides 118 to 162 of SEQ ID NO:2) and has the amino acid sequence of WYLQKPGQSPQLLIY (amino acids 40 to 54 of SEQ ID NO:1); and FR3 of the of the V_{L} region of PH1 is encoded by the nucleotide sequence and reading frame GGG GTC CCT GAC AGG TTC AGT GGC AGT GTA TCA GGC ACA GAT TTT ACA CTG AGA ATC AGC AGA GTG GAG GCT GAG GAT GTT GGA GTT TAT TAC TGC (nucleotides 184 to 279 of SEQ ID NO:2) and has the amino acid sequence GVPDRFSGSVSGTDFTLRISRVEAEDVGVYYC (amino acids 62 to 93 of SEQ ID NO:1).

For the V_{H} region of the PH1 Fab molecule, CDR1 is encoded by the nucleotide sequence and reading frame AGT AAC GCC ATG GGC (nucleotides 91 to 105 of SEQ ID NO:4) and has the amino acid sequence of SNAMG (amino acids 31 to 35 of SEQ ID NO:3); CDR2 is encoded by the nucleotide sequence and reading frame GGT ATT AGT GGT AGT GGT GGC AGC ACA TAC TAC GCA GAC TCC GTG AAG GGC of (nucleotides 148 to 198 of SEQ ID NO:4) and has the amino acid sequence of GISGSGGSTYYADSVKG (amino acids 50 to 66 of SEQ ID NO:3); and CDR3 is encoded by the nucleotide sequence and reading frame CAT ACC GGG GGG GGC GTT TGG GAC CCC ATT GAC TAC (nucleotides 295 to 330 of SEQ ID NO:4) and has the amino acid sequence of HTGGGVWDPIDY (amino acids 99 to 110 of SEQ ID NO:3). FR1 of the V_{H} region of PH1 is encoded by the nucleotide sequence and reading frame CAG GTC CAG CTG GTG CAG TCT GGG GGA GGC TTG GTA CAG CCT GGG GGG TCC CTG AGA CTC TCC TGT GCA GCC TCT GGA TTC ACG TTT AGA (nucleotides 1 to 90 of SEQ ID NO:4) and has the amino acid sequence of QVQLVQSGGGLVQPGGSLRLSCAASGFTFR (amino acids 1 to 30 of SEQ ID NO:3); FR2 of the V_{H} region of PH1 is encoded by the nucleotide sequence and reading frame TGG GTC CGC CAG GCT CCA GGG AAG GGG CTG GAG TGG GTC TCA (nucleotides 106 to 147 of SEQ ID NO:4) and has the amino acid sequence of WVRQAPGKGLEWVS (amino acids 36 to 49 of SEQ ID NO:3); and FR3 of the of the V_{H} region of PH1 is encoded by the nucleotide sequence and reading frame CGG TTC ACC ATC TCC AGA GAC AAT TCC AAG AAC ACG CTG TAT CTG CAA ATG AAC AGC CTG AGA GCC GAG GAC ACG GCC GTA TAT TAT TGT GCG AAA (nucleotides 199 to 294 of SEQ ID NO:4) and has the amino acid sequence RFTISRDNSKNTLYLQMNSLRAEDTAVYYCAK (amino acids 67 to 98 of SEQ ID NO:3).

By indirect epitope fingerprinting (Henderikx et al., Cancer Res., 58: 4324-32 (1998)), the minimal binding epitope in the VNTR of the protein core of MUC1 for the PH1 Fab antibody molecule was determined to have the tripeptide amino acid sequence of Pro Ala Pro. The PH1 Fab bound 3T3-MUC1 cells (expressing MUC1). In BIAcore binding studies using an 80-mer MUC1 core peptide (i.e., four core protein repeat units of a polypeptide having the 20 amino acid sequence of SEQ ID NO:7) as the antigen binding member, PH1 exhibited a slower off-rate (k_{off}=1×10⁻³ sec⁻¹) than other anti-MUC1 scFv antibody molecules, such as scFv-10A (k_{off}=1×10⁻² sec⁻¹), previously retrieved from a scFv phage library (Henderikx et al., Cancer Res., 58: 4324-32 (1998)).

Affinity Maturation of PH1 Fab Antibody MUC1-Binding Site

The PH1 Fab antibody was evaluated for affinity for its MUC1 epitope by surface plasmon resonance (SPR) using a BIAcore 2000 apparatus (BIAcore AB, Uppsala, Sweden) in which the surface of a biotin chip was coated with a MUC1 60-mer peptide antigen (NH₂-(VTSAPDTRPAPGSTAPPAHG)₃-COOH (i.e., containing three copies of SEQ ID NO:8 (von Mensdorff-Pouilly et al., Tumor Biol., 19: 186-195 (1998)). By this analysis, the affinity of the PH1 Fab antibody was determined as a dissociation constant (Kd) for the MUC1 60-mer peptide antigen to be 1.4 micromolar (μM). According to the invention, the intrinsic affinity of a monovalent Fab antibody, such as the monovalent PH1 Fab antibody, for its MUC1 epitope can be improved, for example, by using an in vitro affinity maturation procedure involving phage display to select variants (mutants) of a parent Fab antibody (e.g., PH1 Fab) that bind MUC1, preferably with higher affinity. Details of an actual example of affinity maturation of the PH1 Fab binding site are provided in Example 2, below.

Using affinity maturation and phage display, variants of the PH1 Fab antibody were selected. A list of representative variants of PH1 Fab antibody obtained in one selection (Example 2), is provided in Table 9 (below), which shows that the listed variants contained mutations in the FR3-CDR3 region of the parent PH1 Fab antibody. Dissociation constants (Kds) were calculated for the variants by BIAcore analysis of affinity for the MUC1 60-mer peptide antigen. The results indicated that the affinity of the selected variants for the MUC1 60-mer peptide antigen ranged from about 400 nanomolar (nM), i.e., a 3.5-fold improvement in the PH1 Fab affinity, to about 1.4 μM, i.e., similar to the parent PH1 Fab affinity.

Other MUC1-Specific Binding Member Molecules

In addition to the MUC1-specific Fab antibodies described above, the invention provides other MUC1-specific binding members. The availability of polynucleotide and amino acid molecules encoding specific V_{H} and V_{L} regions of one MUC1-specific binding molecule, such as the PH1 Fab antibody, along with the knowledge of the specific FR and CDR sequences within each variable region of the molecule provide the means for producing any of a variety of other MUC1-specific binding members, or portions thereof, using recombinant DNA procedures or in vitro peptide synthesis protocols. For example, a DNA molecule encoding the antigen binding domain of the PH1 Fab antibody, or portion thereof (such as V_{L}, V_{H}, or one or more CDRs), can be inserted into vectors for expressing new MUC1-specific binding members with the specificity or binding properties of the parent PH1 Fab antibody. Such additional MUC1-specific binding members may include, but are not limited to, full-length immunoglobulin molecules (such as, IgG, IgM, IgA, IgE), other Fab antibodies, F(ab′)₂ antibodies, diabodies, scFv molecules, double-scFv molecules, Fv molecules, domain antibody (dAb) molecules, immunocytokines, and immunotoxins.

MUC1-specific immunoglobulins may be produced by cloning polynucleotides encoding the V_{H} and V_{L} regions of the PH1 Fab antibody into any eukaryotic expression systems available in the art for producing immuoglobulin light and heavy chains, which then assemble into a whole immunoglobulin molecule. An example of such an expression system uses the vectors, VH express (encoding the human gamma-1 heavy constant region) and VKexpress (encoding the human kappa constant domain) (Persic et al., Gene, 187: 9-18 (1997)). Details of a working example of using these expression vectors to produce a completely human, recombinant, MUC1-specific IgG1 antibody (“PH1-IgG1”) from DNA encoding the V_{H} and V_{L} regions of the PH1 Fab antibody are provided in Example 3, below. The PH1-IgG1 comprises an immunoglobulin kappa light chain (V_{L} and C_{L} light chain constant region) having the amino acid sequence of SEQ ID NO:24, which is encoded by the nucleotide sequence of SEQ ID NO:25, and an immunoglobulin heavy chain (V_{H} and heavy chain constant region) having the amino acid sequence of SEQ ID NO:26, which is encoded by the nucleotide sequence of SEQ ID NO:27. BIAcore analysis using the MUC1 60-mer peptide antigen indicated that the PH1-IgG1 molecule exhibited a 100-fold higher apparent Kd (8.7 nM) compared to the Kd of the parent PH1 Fab (1.4 μM). This improved affinity was due to the presence of the two identical MUC1 binding sites of PH1.

The recombinant, human PH1-IgG1 antibody specifically recognizes tumor cells expressing the peptide core epitope of MUC1 of breast and ovarian cancer cell lines, but not colon cancer cell lines, which have heavily glycosylated MUC1 on their surface. Immunohistochemical analysis of PH1-IgG1 indicated that this immunoglobulin intensely stained (i.e., bound) tumor tissue in mammary, ovary, bladder, and lung tissue. In addition, PH1-IgG1 was internalized rapidly into vesicles by human ovarian carcinoma cell line OVCAR-3 cells (see, Example 3). The tumor-associated binding characteristics, the internalization behavior in cancer cells, and the completely human nature of the recombinant, PH1-IgG1 molecule make this molecule, and molecules like PH1-IgG 1, particularly well-suited for use immunotherpeutic, immunodiagnostic, and immunoimaging compositions and procedures. For example, various drugs, polypeptides, and detectable labels (such as, toxins or cytokines, radiolabels or other detectable signals, epitope tags, and imaging compounds) may be conjugated to a MUC1-specific immunoglobulin molecule, such as PH1-IgG1, using standard recombinant DNA methods or in vitro conjugation procedures. The resulting variant is a MUC1-specific immuoglobulin linked to an additional moiety that provides an additional function or label. Such variants can be used as MUC1-specific reagents in various procedures directed or targeted at cancer cells and tissue, especially those directed to tumors found in breast, ovarian, bladder, and lung adenocarcinoma.

In addition, variants of recombinant immunoglobulins may also be prepared from all or a portion of the V_{H} and V_{L} regions from other MUC1 binding members, such as Fab antibodies having improved affinities over the parent PH1 molecule (see, Table 9 and Example 2).

It is also understood that the MUC1-specific immunoglobulins of the invention encompass MUC1-specific immunoglobulin variants, which contain variations in the constant heavy chains of the immunoglobulin molecule, including isotypic variants, such as gamma-1, 2, 3, and 4 isotypes or the alpha-1 and 2 isotypes, and allotypic (intraspecies allelic) variants, such as allotypic variants of gamma-1 or of another isotype.

The V_{H} and V_{L} coding sequences have also been reformatted into a plasmid vector to produce an anti-MUC1 diabody molecule, designated bivPH1. As with all diabodies, bivPH1 is normally (physiological conditions) a dimer of two monomers, each having the motif “V_{H}-L-V_{L}”, where the linker peptide L is a short peptide (for bivPH1, a pentapeptide having the amino acid sequence of G G G A L (amino acids 122 to 126 of SEQ ID NO:5), which restricts intramolecular formation of the MUC1 binding domain from the V_{H} and V_{L} regions. The presence of the linker peptide drives dimer formation resulting in the intermolecular recreation of two MUC1 binding domains. Thus, each bivPH 1 diabody dimer is a bivalent antibody capable of binding to two identical epitopes of a MUC1 core protein VNTR sequence. The anti-MUC1 diabodies of this invention may bind at two identical epitopes in a single MUC1 protein or at the same epitope on two separate MUC1 molecules. Such binding properties are used to advantage in various therapeutic, diagnostic (including imaging), and purification methods described herein.

The invention provides proteins, polypeptides, or peptides that bind MUC1 or that form all or part of a MUC1 binding domain (such as a V_{L}, V_{H}, or one or more CDRs). Such proteins include fusion proteins that are formed by fusing a selected protein of interest to a MUC1-specific binding member, or portion thereof, such as a V_{L}, V_{H}, or CDR(s) from the PH1 Fab antibody described herein. The selected protein of interest may provide the fusion protein with an additional domain useful for purification, diagnostic, or therapeutic application. Thus, the protein of interest for use in a fusion protein of the invention may be any protein, or portion thereof, that can be fused, for example, by recombinant DNA methods, to a MUC1-specific binding member, or portion thereof, described herein and that retains its useful function, activity, or other property in the fusion protein. An example of a fusion protein of the invention is an immunotoxin comprising a MUC1-specific binding portion, such as the bivPH1 diabody, and a toxin portion, which will be toxic to MUC1-expressing tumor cells. Another example of a fusion protein of the invention is an immunocytokine comprising a MUC1-specific binding portion, such as the bivPH1 diabody, and an active cytokine portion, such as IL-2, as described below.

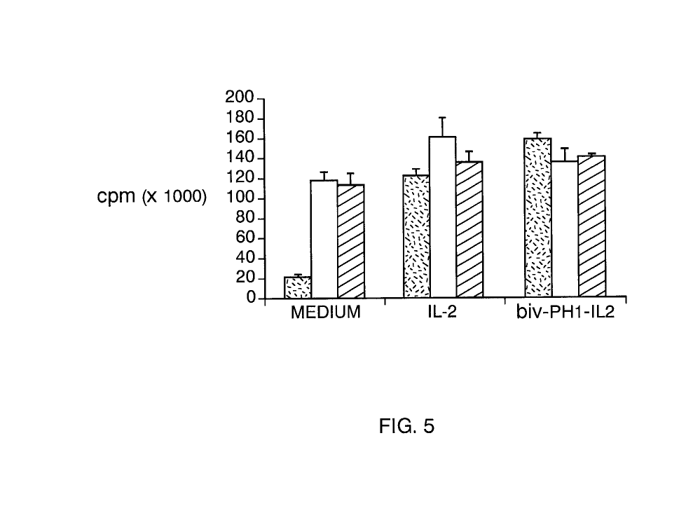

In a further construction, IL-2 was fused to bivPH1 diabody to form a fusion protein, which is an immunocytokine molecule, designated bivPH1-IL-2. The bivPH1-IL-2 has IL-2 immunostimulatory activity as demonstrated by the ability to stimulate peripheral blood lymphocytes (PBL) to lyse cells of the ovarian carcinoma cell line OVCAR-3 in a standard ⁵¹Cr-release assay. In this assay, the bivPH1 diabody did not stimulate lysis by PBL, although the addition of rIL-2 produced a significant increase in killing. The bivPH1-IL-2 immunocytokine enhanced lysis of the OVCAR-3 target cells by the PBL more than the level seen in mixtures of bivPH1 diabody and rIL-2 (see, FIG. 5). Surprisingly, complete killing of tumor cells was achieved using the bivPH1-IL-2 immunocytokine in combination with rIL-2 (FIG. 5).

The bivPH1-IL-2 immunocytokine is a representative of MUC1-specific immunocytokines that comprise a specific MUC1 binding portion fused (conjugated) to an immunomodulatory portion comprising an immunomodulatory protein or peptide, such as a cytokine. The amino acid sequence of bivPH1-IL-2 is shown in SEQ ID NO:5 and a nucleotide sequence encoding the bivPH1-IL-2 immunocytokine is shown in SEQ ID NO:6. Thus, other cytokines could be substituted for the IL-2 immunomodulatory moiety in bivPH1-IL-2, including, but not limited to, GM-CSF and TNF. The MUC1-specific immunocytokines of the invention provide a safer or more efficient means of employing cytokines in cancer therapy because the immunocytokine molecule is able to specifically target MUC1-expressing cancer cells for delivery of the cytokine. The dosage levels used to see an anti-cancer effect with an unconjugated (free) cytokine may also result in a number of undesirable side effects that may even be life-threatening. However, MUC1-specific immunocytokines described herein offer a means for using a cytokine at a relatively low or less toxic dosage level to achieve a therapeutic anti-cancer benefit compared to treatment methods that employ the free cytokine alone.

MUC1-specific immunocytokines may be readily produced by using recombinant DNA techniques in which the V_{H} and V_{L} coding sequences for the PH1 Fab antibody molecule are cloned into a diabody expression vector that also provides a site for the insertion and fusion of a coding sequence for the cytokine of interest, as was done for IL-2 (see, Examples for details). Such immunocytokine fusion proteins are particularly useful for targeting MUC1-expressing cancer cells for killing by a lymphocyte population. The therapeutic effect of using an immunocytokine, such as bivPH1-IL-2, may be further enhanced by additionally administering an unconjugated form of a cytokine (free cytokine), or other compounds, to counteract an anergic or suppressor effect on T cells that is often seen in the area of cancer cells or to augment the anti-tumor effect.

The immunocytokine bivPH1-IL-2 is also an example of the various types of antibody molecules, other than the PH1 Fab antibody, that are provided by the invention which comprise the V_{L} region and/or V_{H} region of the PH1 Fab antibody (SEQ ID NOS:1 and 3, respectively), or may contain one or more CDRs of the PH1 Fab antibody described herein.

The MUC1 binding members of the invention also include derivative proteins that contain amino acid changes (deletions, additions or substitutions) that do not significantly diminish or destroy the MUC1 binding property as described for the various examples of MUC1 binding members provided herein. Such changes in the amino acid sequence of a MUC1 binding member include, but are not limited to, what are generally known as conservative amino acid substitutions, such as substituting one or more amino acids of a V_{H}, V_{L}, CDR, FR, and/or bivPH1-IL-2 amino acid sequence (for example, SEQ ID NOS:1, 3, and 5) with another of similar structure, charge, or hydrophobicity. Any addition or substitution to a MUC1-specific binding member amino acid sequence that maintains MUC1 binding, but also improves another property, such as stability in vivo or in situ, is also useful in the diagnostic, purification, or therapeutic methods of this invention.

An analysis of the PH1 Fab antibody revealed that the heavy chain variable (V_{H}) region is a V_{H} region of the DP47 human germ line and that the light chain variable (V_{L}) region is a V_{L} region of the DPK15 human germ line (see, Example 1, Table 2). Accordingly, the invention also provides MUC1-specific binding members comprising a MUC1-specific binding domain, which binding domain comprises a V_{H} and/or a V_{L} region, or portion thereof (e.g., one or more CDRs), which is encoded on a polynucleotide sequence of the DNA from the DP47 and/or the DPK15 human germ lines.

Furthermore, one or more of the CDRs described herein may be inserted into the FRs from other known germ lines or other cloned antibody domains for cloning and expressing V_{L} and/or V_{H}, or portions thereof, for example using various recombinant DNA methods, to produce additional forms of MUC1-specific antibody molecules.

The invention also provides an isolated MUC1-specific binding member comprising an antigen binding domain, wherein the antigen binding domain comprises an amino acid sequence of the formula:

X₁ X₂ His Thr Gly X₃ Gly Val Trp X₄ Pro X₅ X₆ X₇ (SEQ ID NO:28),

wherein

X₁ is Ala, Ser, Thr, or Val;

X₂ is Lys, Ile Arg, or Gln;

X₃ is Gly, Arg, Val, Glu, Ser, or Ala;

X₄ is Asp or Asn;

X₅ is Ile, Leu, Met, Phe, or Val;

X₆ is Asp, Gly, Lys, Asn, Ala, His, Arg, Ser, Val, or Tyr; and

X₇ is Tyr, His, Lys, Asn, Asp, Ser, Pro.

Preferably, the MUC1-specific binding member comprises the amino acid sequence selected from the group consisting of:

| | |
|---|---|
| Ala Lys His Thr Gly Gly Gly Val Trp Asp Pro Ile Asp Tyr (amino acids 97-110 of SEQ ID NO:3); | |
| Ala Lys His Thr Gly Arg Gly Val Trp Asp Pro Ile Gly Tyr | (SEQ ID NO:29); |
| Ala Lys His Thr Gly Gly Gly Val Trp Asp Pro Ile Lys His | (SEQ ID NO:30); |
| Ala Lys His Thr Gly Gly Gly Val Trp Asp Pro Ile Gly Tyr | (SEQ ID NO:31); and |
| Ala Ile His Thr Gly Gly Gly Val Trp Asp Pro Ile Lys Tyr | (SEQ ID NO:32). |

Such MUC1-specific binding members include any antibody of the various known antibody formats, including immunoglobulin, scFv, double scFv, Fab, F(ab′)₂, Fv, dAb, and diabody antibody formats.

The invention also provides proteins, polypeptides, and peptides comprising amino acid sequences that are not identical, but are homologous, as defined above, to the particular amino acid sequences described herein. In particular, a homologous protein, polypeptide, or peptide useful in the compositions and methods of the invention binds MUC1 or forms all or part of a MUC1-specific binding domain and comprises an amino acid sequence that is about 70%, preferably about 80%, and more preferably about 90% or more (including about 95%, about 97%, or even about 99% or more) homologous to an amino acid sequence for a MUC1-specific binding member, V_{L}, V_{H}, CDR, or portions thereof, described herein.

As mentioned above, the invention also provides MUC1-specific binding members that are variant forms of other MUC1-specific binding members linked to additional domains or molecules, which provide a desirable activity or property. Such variant forms may be formed by linking, preferably covalently, a MUC1-specific binding member molecule described herein to a moiety, such as one or more other proteins or molecules including, but not limited to, a cytokine, a receptor protein, a toxin (e.g., doxorubicin and related drugs, diphtheria toxin, anthrax toxin), an epitope tag (such as a hemagglutinin, polyhistidine, or myc epitope tag), a fluorescein dye, streptavidin, biotin, an enzyme (e.g., horseradish peroxidase (HRP), β-galactosidase, or a site-specific protease), or a radioactive compound, such as ¹²⁵I or ^{99m}Tc, and the like. Linkage of the moiety to the MUC1-specific binding member may involve the use of “linker molecule or peptide” that connects the binding member to the moiety. Such variants find use in purification, diagnostic, imaging, and therapeutic methods of the invention, particularly those directed to adenocarcinoma tumors in mammary, ovary, bladder, and lung tissue.

The invention also provides isolated polynucleotide molecules that encode an amino acid sequence for the various proteins, polypeptides, and peptides described herein that bind MUC1 or that form all or part of a MUC1 binding domain (such as a V_{L}, V_{H}, or a CDR). Such polynucleotide molecules may be DNA or RNA (wherein in RNA contains uracil instead of thymine).

Polynucleotide molecules of the invention also comprise degenerate sequences, i.e., nucleotide sequences that differ from sequences specifically listed herein in that they contain different codons that code for the same amino acid according to the genetic code, and therefore encode the same protein, polypeptide, or peptide, e.g., MUC1-specific binding member, V_{L}, V_{H}, and/or portions thereof such as CDRs and FRs.

Polynucleotide molecules of the invention also include polynucleotide molecules that have nucleotide sequences that are homologous, as defined above, to the particular sequences described herein (e.g., SEQ ID NOS:2, 4, 6, 25, and 27). In one embodiment, a homologous polynucleotide molecules of the invention may comprise a nucleotide sequence that is about 60%, preferably about 70%, more preferably about 80%, and even more preferably 90% or more, homologous to a nucleotide sequence described herein and encodes a MUC1-specific binding member, a MUC1-binding domain, or portion thereof (such as a CDR). A homologous polynucleotide molecule of the invention may also comprise a degenerate polynucleotide sequence as described above.

Isolated nucleic acid molecules, especially DNA molecules, of the invention comprise nucleotide sequences that encode all or a portion of the MUC1 binding domain of the PH1 Fab antibody, including the V_{L} and/or V_{H} regions of PH1 (SEQS ID NOS:2 and 4, respectively), or one or more CDRs and/or FRs of the V_{L} or V_{H} regions. The nucleic acid molecules of the invention, which comprise a nucleotide sequence encoding a MUC1 binding member or MUC1 binding domain, or portion thereof, may be in a variety of forms, including but not limited to, plasmids, which include cloning and expression plasmid vectors used in prokaryotes; phage genomes or phagemids, which include lysogenic phages that may integrate into the bacterial chromosome; eukaryotic expression and cloning plasmid or viral vectors; linear nucleic acid molecules, which include linear DNA or RNA molecules, such as mRNA molecules; and synthetically made nucleic acid molecules.

The various nucleic acid molecules described above may be used to produce MUC1-specific binding members of the invention using recombinant nucleic acid methodologies. For example, nucleic acid molecules comprising nucleotide sequences described herein may be combined or synthesized in vitro using standard cloning methods or chemical synthesis to encode any of the MUC1-specific binding members of the invention and then inserted into an appropriate expression vector, such as an expression plasmid, phagemid, or other expression viral vector. A nucleic acid molecule having a sequence encoding the MUC1-specific binding member must be operably linked to a promoter in the expression vector. The recombinant expression vector containing the coding sequence for the MUC1-specific binding member is then placed or inserted, e.g., by transformation, transfection, electroporation, into an appropriate host cell that will express the MUC1-specific binding member encoded on the vector. The host cell may be a prokaryotic or eukaryotic cell depending on the type of expression vector used.

In addition, a nucleic acid molecule encoding a MUC1-specific binding member may be operably linked in a display vector to an anchor sequence, which encodes all or part of a surface protein, so that the expressed MUC1-specific binding member is displayed on the surface of a particular genetic package, i.e., a phage or cell, which includes, but is not limited to, M13-derived phage, M13-derived phagemids, and yeast cells (see, e.g., Vanlntwerp et al., Biotechnol. Prog., 16: 31-37 (2000); Wittrup, Trends In Biotechnol., 17: 423-424 (1999); Kieke et al., Proc. Natl. Acad. Sci. USA, 96: 5651-5656 (1999)). Such display systems are useful for mutagenizing a gene segment encoding a MUC1-specific binding member (e.g., by introducing alternative CDR sequences) to produce a population of genetic packages, each carrying one member of a family of variant genes and displaying that variant MUC1-specific binding member. From the population of displayed variants, individual variants having a superior property, such as an enhanced avidity or affinity for MUC1, can then be selected by methods known in the art. Preferably, enhancing affinity (affinity maturation) of a MUC1-binding member is carried out using a yeast display vector and an appropriate yeast host cell.

Any of the various polynucleotide molecules of the invention described herein also find use as probes for genes encoding MUC1-specific binding proteins or portions thereof, including alleles or mutated gene sequences encoding corresponding allelic or variant forms of a MUC1-specific binding iprotein or portion thereof.

Diagnostic, Purification, and Therapeutic Methods of Use

The MUC1-specific binding members of the invention may be used in methods for diagnosing and imaging MUC1-expressing cancer cells and tissue; for purifying or isolating non-glycosylated, underglycosylated, or cancer-associated forms of MUC1 or MUC1 epitope-containing molecules; and/or for therapeutically or prophylactically treating (i.e., antibody-based passive immunotherapy) MUC1-expressing cancer, such as adenocarcinoma.

For diagnosing cancer, such as adenocarcinoma, a sample, such as cells, tissues (e.g., biopsy sample), and/or body fluid (e.g., bone marrow, urine, and/or blood) obtained from an individual is contacted with a MUC1-specific binding member described herein. As noted above, the MUC1-specific binding members of this invention comprise a V_{L} and/or V_{H} region, or portion thereof (such as CDRs), which forms a binding domain for an epitope in the VNTR of the MUC1 protein core. Thus, the diagnostic methods described herein may be used to test for evidence of cancer in an individual by detecting binding of a MUC1-specific binding member of this invention to MUC1-expressing cells or tissues or to MUC1 present in blood or other fluid of an individual. Such diagnostic methods may be performed completely in vitro, as with many standard clinical diagnostic tests. Alternatively, a diagnostic procedure may be performed in vivo and involve the administration of a MUC1-specific binding member to a individual. The binding of the administered MUC1-specific binding member to cells or tissues may then be detected either in vivo (e.g., by imaging methods) or in vitro.

A variety of detection systems are available to detect antibody bound to an antigen on cells or tissues or in a fluid, and such detection systems may be employed by the skilled practitioner in the diagnostic methods of this invention to detect bound MUC1-specific binding member. The detection of a bound MUC1-specific binding member will usually involve detecting a signal from a label or tag linked or bound either directly to the MUC1-specific binding member or to a separate detection molecule, which in turn will bind to a MUC1-specific binding member. Whether linked directly to the MUC1-specific binding member or to a separate detection molecule, such labels or tags that are useful in the diagnostic methods of this invention include, without limitation, enzymes, fluorescent labels, radioactive labels, heavy metals, and magnetic resonance imaging (MRI) labels, such as used for diagnostic tumor imaging. If the label is an enzyme, the binding can be detected by using a substrate that produces a detectable signal, such as a colorigenic, bioluminescent, or chemiluminescent substrate. Enzyme label detection systems include those using the biotin-streptavidin (or avidin) pair, for example, in which the MUC1-specific binding member or a detection molecule is conjugated to biotin (or streptavidin) which in turn will bind to streptavidin- (or biotin) conjugated to an enzyme of the detection system, such as β-galactosidase, horseradish peroxidase, or luciferase. For example, a detection antibody linked to a label or tag, such as an enzyme or radioactive label, may also be used to detect a MUC1-specific binding member that has bound to MUC1 on the cells or tissues or in the blood or fluid of an individual. The label or tag on the detection antibody is then detected to determine the amount of and/or location of the bound MUC1-specific binding member. Various methods for detecting such labeled or tagged molecules are well known to those skilled in the art and include, without limitation, enzyme-linked immunosorbent assay (ELISA) or immunoprecipitation protocols. Such methods may employ fully or semi-automated devices to more efficiently read and process multiple test samples. If the label or tag is radioactive, the detection means is anything that is sensitive to the radioactivity, such as, X-ray film, scintillation counters, Geiger counters, or body imagining or scanning devices, such as magnetic resonance imagining (MRI) machines.

The MUC1-specific binding members of this invention may also be used to purify or extract MUC1 protein molecules in a mixture or sample. Procedures that use antibodies for isolating or purifying an antigen may be adapted by substituting a particular MUC1-specific binding member of the invention for the conventional antibody component. Such procedures include without limitation direct binding to MUC1 molecules in solution followed by precipitation, such as in immunoprecipitations, ELISA, and affinity chromatography. For affinity chromatography, resins may be prepared in which a MUC1-specific binding member of this invention is conjugated to resin particles using methods already established for conjugating immunoglobulins and other binding proteins. As with any affinity resin, the ability to bind a cognate partner or ligand on the resin, such as MUC1 molecules, will depend on the availability of exposed MUC1 epitopes on the resin particles after conjugation of the specific binding member to the resin.

The MUC1-specific binding members described herein may also be used as therapeutic or prophylactic reagents to treat cancer, such as adenocarcinoma. MUC1-specific binding members provided herein may be used either in an unmodified form, or as a variant in which a MUC1-specific binding member is bound to, conjugated to, or engineered as a fusion protein to possess another moiety having an effector function that would damage or kill cancerous cells or tissues or that would stimulate or promote an anti-tumor immune response. Thus, the invention provides therapeutic and prophylactic methods of treating cancer, especially adenocarcinoma, in an individual. The methods of treating cancer according to the invention include both in vivo and ex vivo methods.

One method of treating adenocarcinoma in an individual comprises administering to the individual a completely human, recombinant, MUC1-specific immunoglobulin, such as PH1-IgG1 (see, Example 3). Preferably, the immunoglobulin is also linked to another moiety that provides an anti-cancer function, such as an anti-cancer compound or cell toxin, which only is toxic to cells that bind and internalize the MUC1-specific immunoglobulin.

In another treatment method, certain cells are delivered to a MUC1-expressing cancer tumor or cancerous tissue using a MUC1-specific binding member of the invention. To deliver cells, such as T cells or killer cells to a MUC1-expressing tumor or tissue, a MUC1 binding member may be conjugated or fused to another binding domain, such as a receptor, that specifically binds a marker antigen on the surface of the particular cells to be delivered, so that the resultant MUC1 binding member now binds to both MUC1 and the cells to be delivered.

A MUC1-specific immunocytokine of this invention, such as the bivPH1-IL-2 immunocytokine, which is a fusion protein containing an active IL-2 domain, may be administered to an individual to target the IL-2 immunostimulatory function to cancer cells in the body in order to promote a T cell-mediated anti-tumor immune response. The anti-tumor immune response may be further enhanced by also administering one or more doses of a nonconjugated form of the same or related cytokine, for example, recombinant IL-2, or another more preferred immunostimulatory compound. Such a supplemental or augmentation dose of a nonconjugated cytokine or other compound may be administered prior to, contemporaneously with, or subsequently to administering the MUC1-specific binding member to the individual.

A MUC1-specific binding member of this invention may be used alone or as a component in a more complex anti-cancer regimen, which may include one or more other anti-cancer drugs and/or radiation treatments. Also, multiple treatments may be administered to an individual. Preferably, the particular MUC1-specific binding member used for multiple administrations is a protein or polypeptide molecule of human source, such as PH1 Fab, bivPH1-IL-2, or PH1-IgG1 antibody, so that the individual's immune system does not raise antibodies that would inactivate or rapidly clear the MUC1-specific binding member from the body.

Thus, MUC1-specific binding members described herein, may be used to target a wide variety of anti-tumor effector functions to tumors or pre-cancerous cells and tissues including, but not limited to, the immunomodulatory activity of a cytokine, such as IL-2; an anti-cancer drug; a toxin; a radioactive compound; T cells; killer cells; heavy metals; and other anti-cancer molecules.

The MUC1-specific binding members of the invention may also be used in ex vivo methods for treating cancer, which deplete or purge MUC1 and MUC1-expressing cancer cells from cells, tissues, or body fluids, such as bone marrow, blood, or peripheral blood stem cells. For example, in one preferred embodiment, the ex vivo method of cancer treatment comprises extracting a body fluid containing MUC1 and/or MUC1-expressing cancer cells from an individual and contacting the extracted body fluid with a MUC1-specific binding member. Preferably, the MUC1-specific binding member is immobilized on a solid support or surface. The body fluid so treated is thereby depleted or purged of the MUC1 and/or MUC1-expressing cancer cells and returned to the individual. More preferably, the ex vivo methods of treating cancer of the invention comprise using an immobilized MUC1-specific binding member comprising an amino acid sequence selected from the group consisting of SEQ ID NO:1; amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQ ID NO:3, amino acids 50 to 66 of SEQ ID NO:3, amino acids 99 to 110 of SEQ ID NO:3, conservatively substituted versions of any of the preceding sequences, and combinations thereof. A variety of systems are available that may be used to immobilize a MUC1-specific binding member to a surface. Such systems may involve direct or indirect conjugation of a MUC1-binding member to a solid surface such as plastic, Sepharose, magnetic or paramagnetic beads, or various other resins. The body fluid taken from an individual may be contacted with the immobilized MUC1-specific binding member in a batch protocol or using a column or other surface containing the immobilized MUC1-specific binding member. Immobilization of the MUC1-specific binding member may be done before, during or after the addition of the cells, tissues, or body fluid taken from an individual. The ex vivo methods of the invention may employ automated, semi-automated, or manually operated devices. In addition, body fluid may be contacted with the immobilized MUC1-specific binding member in a non-continuous or continuous flow system. Furthermore, the extracted body fluid must be kept from contamination and may be further treated to prevent or eliminate contamination by undesirable cells, viruses, chemicals, and/or antigens. In another embodiment, one or more anti-cancer agents, antibiotics, or other therapeutic compounds are added to the depleted or purged body fluid prior to its return to the individual. Such anti-cancer agents may include an MUC1-specific binding member described herein.

Pharmaceutical Compositions and Modes of Administration

A MUC1-specific binding member is preferably administered to an individual (human or other animal) in a “therapeutically effective amount”, which is understood to mean an amount that is sufficient to show a benefit to a patient. Such a benefit may be at least an amelioration of at least one symptom of a cancer, such as adenocarcinoma, including but not limited to, death of tumor cells, stasis of tumor growth, decrease in development of tumor size, decrease in or prevention of metastasis, increase in patient strength or vigor, healthy tissue weight gain, prolongation of survival time, and absence of relapse.

The actual amount administered, and rate and time-course of administration, will depend on the nature and severity of the cancer being treated. Prescription of treatment and selection of dosages to use for a patient are within the knowledge and responsibility of the skilled healthcare practitioner. In addition, appropriate doses of immunoglobulin antibody molecules are well known in the art and provide guidance for deciding on a dose or range of doses of MUC1-specific binding members of this invention to be used in a particular therapeutic regimen (see, e.g., Ledermann et al., Int. J. Cancer, 47:

659-664 (1991); Bagshawe et al., Antibody, Immunoconjugates and Radiopharmaceuticals, 4: 915-922 (1991)).

Pharmaceutical compositions or medicaments according to the present invention comprise at least one MUC1-specific binding member provided by the invention as an active ingredient and may comprise, in addition to the active ingredient, a pharmaceutically acceptable excipient, carrier, buffer, stabilizer, or other materials that are well known to those skilled in the art. Such materials should be non-toxic and should not interfere with the efficacy of the active ingredient. The precise nature of the carrier or other material will depend on the route of administration, which may be oral, topical, or parenteral, e.g,, by intravenous or intramuscular injection.

The pharmaceutical compositions or medicaments provided by the invention may be prepared in any of a variety of forms particularly suited for the intended mode of administration, including solid, semi-solid or liquid dosage forms, for example, tablets, lozenges, pills, capsules, powders, suppositories, liquids, aqueous or oily suspensions, liposomes or polymer microcapsules or microspheres, syrups, elixirs, and aqueous solutions. Preferably, the pharmaceutical composition is in a unit dosage form suitable for single administration of a precise dosage, which may be a fraction or multiple of a dose, which is calculated to produce an effect on adenocarcinoma tumor cells or the T cell-mediated anti-tumor response of the patient. The compositions will include, as noted above, a therapeutically effective amount of a selected MUC1-specific binding member in combination with a pharmaceutically acceptable carrier and/or buffer, and, in addition, may include other medicinal and anti-cancer agents or pharmaceutical agents, carriers, diluents, fillers and formulation adjuvants, or combinations thereof, which are nontoxic, inert, and pharmaceutically acceptable. In liquid mixtures or preparations, a pharmaceutically acceptable buffer, such as a phosphate buffered saline may be used. By “pharmaceutically acceptable” is meant a material that is not biologically, chemically, or in any other way, incompatible with body chemistry and metabolism and also does not adversely affect the MUC1-specific binding member or any other component that may be present in the pharmaceutical composition.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Pharmaceutically acceptable liquid compositions can, for example, be prepared by dissolving or dispersing a MUC1-specific binding member as described herein and optimal pharmaceutical adjuvants in an excipient, such as, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, triethanolamine oleate.

For intravenous injection, or direct injection into a tumor or at a site of affliction, the selected MUC1-specific binding member of this invention will preferably be formulated in a parenterally acceptable aqueous solution, which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilizers, buffers, antioxidants and/or other additives may be included, as required. Formulations comprising a MUC1-specific binding member described herein may also be prepared for injection or infusion into an individual using pumps or slow drip devices.

Also within the scope of this invention, a MUC1-specific binding member may alternatively be prepared as a bolus, which may contain a mordant for gradual release from an injection site. One approach for parenteral administration involves use of a slow release or sustained release system, such that a constant level of dosage is maintained (see, for example, U.S. Pat. No. 3,710,795).

Additional embodiments and features of the invention will be apparent from the teaching and guidance provided by the following non-limiting examples of MUC1-specific binding members.

### EXAMPLES

The following examples of the invention describe production and use of MUC1-specific binding members, such as MUC1-specific Fab antibodies, a fully human anti-MUC1 immunoglobulin, and an immunocytokine fusion protein. Such MUC1-specific binding members have an unexpected enhanced avidity for the protein core of MUC1. In addition, MUC1-specific binding members that also comprise an immunomodulatory domain, such as the immunocytokine bivPH-1-IL-2, described below, are able to stimulate T cells and, therefore, counteract MUC1-related inhibition of T cell activation, which is necessary for a T cell mediated anti-cancer immune response

### Example 1

Selection, Characterization, and use of the Cell Binding Fab PH1 Antibody to the Core Protein of MUC1

A MUC1 negative murine fibroblast cell line 3T3 and a MUC1-transfected 3T3 cell line 3T3-MUC1 (Acres et al., J. Immunother., 14: 136-43 (1993)), a biotinylated MUC1 100-mer peptide with the sequence NH₂-(PAHGVTSAPDTRPAPGSTAP)₅-COOH (i.e., containing five copies of the sequence of SEQ ID NO:7) (Krambovitis et al., J. Biol. Chem., 273: 10874-10879 (1998)) and a MUC1 60-mer peptide NH₂-(VTSAPDTRPAPGSTAPPAHG)₃-COOH (i.e., containing three copies of the sequence of SEQ ID NO:8) (von Mensdorff-Pouilly et al., Tumor Biol., 19: 186-195 (1998)) were used during the selection. A large, naive, human Fab library expressed on phage, containing 3.7×10¹⁰ different antibodies (de Haard et al., J. Biol. Chem., 274: 18218-18230 (1999)) was used. Cell selections were carried out as described (Hoogenboom et al., Eur. J. Biochem., 260: 774-84 (1999)) after depletion with a cell line not expressing MUC1. Briefly, adherent, confluent cells were washed twice with PBS (0.15 M NaCl, 8 mM Na₂HPO₄, 7.8 rnM KH₂PO₄, pH 7.2) and subsequently trypsinized (trypsin/EDTA). Cells and human Fab library were preincubated in 2 g skimmed milk per 100 ml PBS (M-PBS). To deplete fibroblast cell binders from MUC1-transfected cell binders, 5×10¹³ phages were preincubated with 5×10⁷ 3T3 cells for 1 hour at room temperature in 5 ml M-PBS. Cells were centrifuged (3 minutes at 611×g), and the supernatant liquid containing the depleted phage library was added to 1×10⁷ 3T3-MUC1 cells for 1 hour at room temperature. Cells were washed 10 times with 5 ml M-PBS/10% fetal calf serum (FCS) and 2 times with PBS. After the last wash, the cell pellet was resuspended in 0.6 ml H₂0 and phages were released from the cells by the addition of 0.6 ml triethylamine (200 mM). The suspension was neutralized with 0.6 ml IM Tris-HCI (pH 7.4) and spun down for 5 minutes at 21,000×g. The supernatant contained the selected phages. Two different selection strategies were compared: 4 rounds of selection on cells or two rounds of selection on cells followed by three more selections on the MUC1 60-mer (to avoid remaining cell binders and/or glycosylated MUC1 binders) as described before (Henderikx et al., Cancer Res., 58: 4324-32 (1998)). The latter selection strategy (selections on MUC1-expressing cells followed by selections on the MUC1 60-mer) yielded the PH1 Fab antibody described herein.

Screening and Characterization of Clones Selected from the Fab Library

Screening and characterization of cell binding clones by whole cell ELISA, fingerprint analysis, flow cytometry, sequencing, indirect epitope fingerprinting and immunohistochemistry was performed according to the methods we described before (Hoogenboom et al., Eur. J. Biochem., 260: 774-84 (1999), Henderikx et al., Cancer Res., 58: 4324-32 (1998)). For screening purposes, individual clones were picked and transferred to 96-well plate and phage was produced as described in (Marks et al., J. Mol. Biol., 222: 581-597 (1991)). Individual clones of rounds 4 and 5 were tested for their specificity by whole cell ELISA (Hoogenboom et al., Eur. J Biochem., 260: 774-84 (1999)) on a MUC1-negative murine fibroblast cell line 3T3 and a MUC1-transfected 3T3 cell line. Clones were considered positive in whole cell ELISA when the A₄₅₀ (horseradish peroxidase staining reaction) of the MUC1-transfected 3T3 cell line was at least 3 times higher than the A₄₅₀ of the MUC1-negative 3T3 cell line. Positive clones were screened for diversity in fingerprint analysis by polymerase chain reaction (PCR), using primer CHIFOR (5′-GTC CTT GAC CAG GCA GCC CAG GGC-3′) (SEQ ID NO:9), from the constant CH1 region of Fab antibodies, and pUC-reverse (5′-AGC GGA TAA CAA TTT CAC ACA GG-3′) (SEQ ID NO:10), followed by BstNI enzyme digestion and analysis of the restriction fragments by agarose gel electrophoresis (Marks et al., J. Mol. Biol., 222: 581-597 (1991), Gussow et al., Nucleic Acids Res., 17: 4000 (1989)). Cell binding of unique positive clones was evaluated by flow cytometric analysis of phage binding pattern (Rousch et al., Br. J. Pharmacol., 125: 5-16 (1998)) on the same cell lines as used during the selection. The V-genes of one Fab, clone PH1, were sequenced using a cycle sequencing kit according to the directions of the manufacturer (Edge Biosystems, Gaithersburg, Md.). Primers were the same as for fingerprinting. Nucleotide sequences and their corresponding deduced amino acid sequences were aligned and compared to the germ line sequences of the Sanger Center Sequence database (http://www.sanger.ac.uk/DataSearch/gq_search.shtml) (Table 2). As shown in Table 2, the V_{H} region of the PH1 Fab antibody is a V_{H} region from the DP47 germ line and the V_{L} region is a V_{L} region from the DPK15 germ line. The selection strategies used here are compared with selections on MUC1 that were previously described (see, Table 1; de Haard et al., J Biol Chem., 274: 18218-18230 (1999), Henderikx et al., Cancer Res., 58: 4324-32 (1998)). Likewise, the further characterization of the clones and constructs was performed by methods previously described (see, Henderikx et al., Cancer Res., 58: 4324-32 (1998)) and are specified only briefly herein.

TABLE 1
Selections for specific MUC1 antibodies with a very large Fab library
on cells compared with previously published selections

| | ScFv library^{a} | | | Fab library^{b} | | |
|---|---|---|---|---|---|---|
| Method of | Nr | Nr Cell | k_{off} (s⁻¹) | Nr | Nr Cell | k_{off} (s⁻¹) |
| selection | Abs | binders | (range) | Abs | binders | (range) |
| MUC1 peptide | 3^{c} | 0 | — | 0 | — | — |
| coated on tubes | | | | | | |
| Biotinylated | 5^{c} | 2 | 10⁻¹-10⁻² | 14^{b} | 0 | 10⁻³-10⁻⁴ |
| MUC1 100- | | | | | | |
| mer | | | | | | |
| MUC1 | 0^{c} | — | — | 0 | 0 | — |
| expressing cells | | | | | | |
| MUC1 | 0 | — | — | 6 | 1 | 10⁻²-10⁻³ |
| expressing | | | | | | |
| cells/coating^{d} | | | | | | |

^{a}(Vaughan et al., Nat Biotechnol., 14:309-314 (1996)), ^{b}(de Haard et al., J Biol Chem., 274:18218-18230 (1999)), ^{c}(Henderikx et al., Cancer Res., 58:4324-32 (1998)), ^{d}coating + cell selection for scFv library or cell selection + coating for Fab library, ^{e}not done

TABLE 2

Deduced amino acid sequence of MUC1 specific antibody PH1 compared with germ line
sequences^{a}

| | | |
|---|---|---|
| | FR1 CDR1 FR2 CDR2 | |
| | 10 20 30 40 50 60 | |
| DP47 | EVQLLESGGGLVQPGGSLRLSCAASGFTFS SYAMS WVRQAPGKGLEWVS AISGSGGSTYYADSVKG | |
| PH1^{a} | q---vq-----------------------R -N-G- -------------- G---------------- | |
| | FR3 CDR3 FR4 | |
| | 70 80 90 | |
| DP47 | RFTISRDNSKNTLYLQMNSLRAEDTAVYYCAK | (SEQ ID NO:18) |
| PH1^{a} | -------------------------------- HTGGGVWDPIDY WGQGTLVTVSS | (SEQ ID NO:3) |
| | FR1 CDR1 FR2 CDR2 | |
| | 10 20 30 40 50 60 | |
| DPK15 | DIVMTQSPLSLPVTPGEPASISC RSSQSLLHSNGYNYLD WYLQKPGQSPQLLIY LGSNRAS | |
| pH1^{a} | e—-1------------------- ------------T--- --------------- S—H--- | |
| | FR3 CDR3 FR4 | |
| | 70 80 90 100 | |
| DPK15 | GVPDRFSGSGSGTDFTLKISRVEAEDVGVYYC MQALQTP | (SEQ ID NO:19) |
| PH1^{a} | ---------V-------R-------------- -------FT FGPGTKVDIKR | (SEQ ID NO:1) |

^{a}lower case, primer encoded mutations; upper case, amino acid mutations. FR, framework region; CDR, complementarity determining region.

Specificity of MUC1 cell binding was tested in flow cytometry on the murine fibroblast cell lines 3T3, the 3T3 MUC1-transfected line ETA, the breast carcinoma line T47D, the ovarian carcinoma line OVCAR-3, and the colon cancer cell line LS174T. The relative amounts of antibodies were compared using dot blots. The same amount of scFv, PH1, and bivPH1, and 3 times less bivPH1-IL-2 was used, as determined in dot blot. MUC1 specificity was confirmed by preincubation of the antibodies with 100 μg/ml of the synthetic MUC1 60-mer for 1 hour at room temperature. Tumor tissue binding was evaluated by immunohistochemistry on paraffin embedded tissues of breast, ovarian and colon carcinoma and normal tissues. Fine specificity was measured by indirect epitope fingerprinting (Henderikx et al., Cancer Res., 58: 4324-32 (1998)).

Generation of a Bivalent Diabody-IL-2 Fusion Protein BivPH1-IL-2, an Immunocytokine MUC1-specific Binding Member

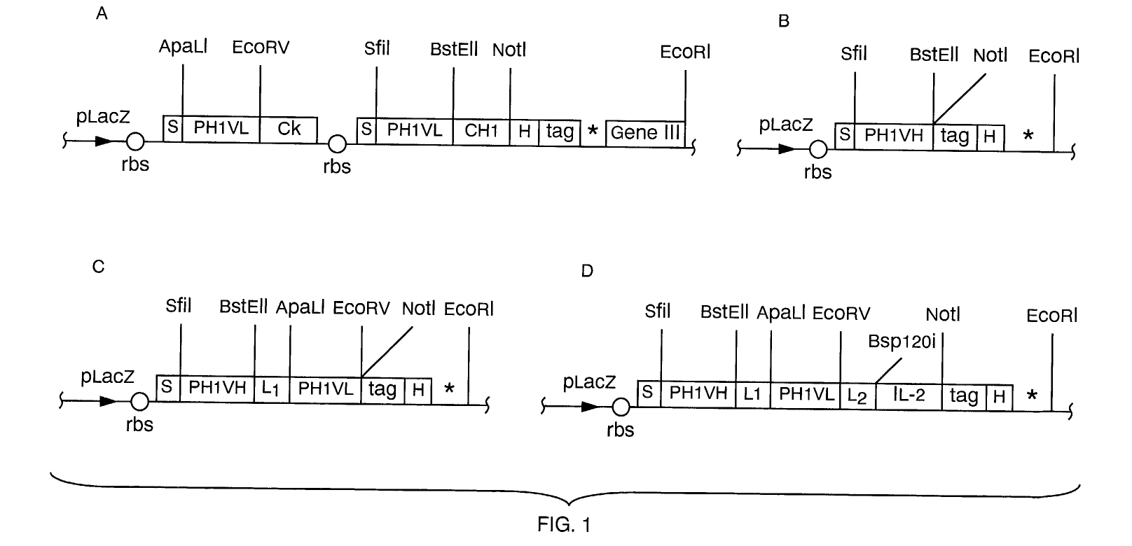

The Fab antibody PH1 was chosen for the construction of a dimeric, bivalent antibody fused to IL-2. The cloning schedule for the immunocytokine into a bacterial expression plasmid is shown schematically in FIG. 1. The first cloning step included the insertion into plasmid pCANTAB6 (McGuinness et al., Nature Biotechnol., 14: 1149-54 (1996)), digested with SfiI and EcoRI, of two fragments: (1) the heavy chain variable region (V_{H}) of PH1 (as SfiI-BstEII restriction fragment), and, (2) a region from the diabody vector pDial (as a BstEII-EcoRI fragment), (Roovers et al., 1999, unpublished) providing the unique restriction site NotI and the myc-tag (GAA CAA AAA CTC ATC TCA GAA GAG GAT CTG AAT GGG GCC GCA (SEQ ID NO:21), encoding the myc-tag amino acid sequence EQKLISEEDLNGAA (SEQ ID NO:20)), and a polyhistidine (“hexaHis”) tag (i.e., CAT CAC CAT CAT CAC CAT (SEQ ID NO:23), encoding the six-histidine amino acid sequence HHHHHH (SEQ ID NO:22)) to yield plasmid pC6-PH1-VH (FIG. 1B). The tags are needed as handles for subsequent detection and purification of the diabody. In a second step (FIG. 1C), a two-step PCR was performed with a first amplification of the V_{L}-C_{L} of the parental Fab PH1 with primers V_{L} backward 35: 5′-ACC GCC TCC ACC AGT GCA CTT GAA ATT GTG CTG ACT CAG TCT CC (SEQ ID NO:11) and V_{L} forward: ACC GCC TCC ACC GGG CGC GCC TTA TTA ACA CTC TCC CCT GTT GAA GCT CTT (SEQ ID NO:12). A second PCR of the V_{L} was performed with primers designed to add a 5 amino acid linker (L1) and restriction sites needed for following cloning steps. A linker of 5 residues favors the folding of scFvs as a diabody (Rousch et al., Br. J. Pharmacol., 125: 5-16 (1998)). The primers were: PH1 V_{L} backward: 5′ GCCGATCGCTCTGGTCACCGTCTCAAGCGGAGGCGGTGCACTTGAAATT GTGCTGACTCAG (SEQ ID NO:13) and PH1 V_{L} forward: 5′ GTCTCGCGAGCGGCCGCCGA TTGGATATCCACTTTGGTCCCAGGGCCGAA) (SEQ ID NO:14). This PCR product was cloned into the pC6-PH1-VH via BstEII/NotI, resulting in plasmid pKaPa1. From this vector, the antibody fragment PH1 will be expressed as a bivalent MUC1 specific diabody bivPH1 (FIG. 1C). In a third step, we fused IL-2 to the diabody construct (FIG. 1D). The template for the PCR amplification of the IL-2 encoding gene was obtained by reverse-transcriptase-PCR (RT-PCR), (kit supplied by Perkin Elmer, Branchburg, N.J.), of total RNA (RNAzol, Campro Scientific, Veenendaal, The Netherlands) derived from PBL stimulated with phytohaemagglutinin (PHA) for 8 h for maximal expression of IL-2 (Fan et al., Clin. Diagn. Lab. Immunol., 5: 335-40 (1998)). The IL-2 gene was inserted in the diabody vector between PH1V_{L} and the tag-encoding fragment (i.e., the myc-tag followed by the six-histidine peptide tag), through NotI/EcoRV, resulting in a phage vector, pKaPa2, encoding a secreted diabody-IL-2 fusion protein (bivPH1-IL-2) (see, FIG. 1D). ScFv-IL-2 fusion proteins with linkers between 4 and 13 residues (Melani et al., Cancer Res., 58: 4146-54 (1998), Savage et al., Br. J. Cancer, 67: 304-10 (1993)) have been described. A nine amino acid encoding linker (GGG GGT GGA TCA GGC GGC GGG GCC CTA) (SEQ ID NO:15) was chosen in order to avoid potential steric hindrance between the two antigen binding sites of the diabody and IL-2 and to minimize enzymatic cleavage. This sequence was primer encoded (PH1-IL-2 backward: 5′ ACCAAAGTGGATATCAAACGAGGGGGTGGATCAGGCGGCGGGGCCCTAGCACCTAC TTCAAGTTCTACA (SEQ ID NO:16); PH1-IL-2 forward: 5′ GTCCCGCGTGCGGCCGCAGT CAGTGTTGAGATGATGCTTTGACAAAAGG) (SEQ ID NO:17)).

BIAcore Analysis of ScFv, Fab, and Bivalent Antibody Fragments

The selected Fab PH1 and other antibody constructs were evaluated by surface plasmon resonance on a BIAcore 2000 apparatus (Pharmacia). A CM-5 chip was coated with the MUC1 80-mer (containing four copies of the amino acid sequence of SEQ ID NO:7) at a density of 90 or 800 response units (RU) in 10 mM acetate buffer at pH 4.6. An empty, activated and subsequently deactivated surface was used as a negative control. The Fab fragment PH1, scFv 10A (Henderikx et al., Cancer Res., 58: 4324-32 (1998)), and the engineered diabody fragments were injected in HBS buffer (Pharmacia, Uppsala, Sweden). To minimize rebinding of the antigen binding fragments, a flow rate of 20 μl/s was used.

Purification of Antibody Fragments

For assays, involving cell culture, antibody fragments were purified by immobilized metal affinity chromatography (Roovers et al., Br. J. Cancer, 78: 1407-16 (1998)). Free IL-2 present in the final product was removed by ultra-filtration against PBS in a centrifugal concentrator (3000 rpm) (Mᵣ cut-off 30 000, Centricon, Millipore, Bedford, Mass.). The volume was reconstituted by the addition of PBS to the maximal capacity of the concentrator and the sample was concentrated again by centrifugation. The reconstitution and concentration was repeated twice. Absence of non-conjugated IL-2 was checked by sodium-dodecyl-sulfate polyacrylamide gell electrophoresis (SDS-page) and Western-blot.

IL-2 Assays

IL-2 concentrations of the bivPH1-IL-2 construct and the IL-2 control (Boehringer, Mannheim, Germany) were quantitated by means of ELISA for the purpose of later use in in vitro stimulation assays. The ELISA was performed following the directions of the supplier (Endogen, Woburn, Mass.). The activity of the bivPH1-IL-2 was measured by stimulation of an IL-2 dependent murine T cell line CTLL-16 (Heeg et al., J. Immunol. Methods., 77: 237-46 (1985), Gillis et al., J. Immunol., 120: 2027-32 (1978)). Cells, cultured in RPMI 1640 (10% FCS, 100 U IL-2 per ml), were washed 3 times. 10⁴ cells per well were incubated with increasing concentrations ranging from 0.2-4 pg/ml of rIL-2 or bivPH1-IL-2 in round bottomed microtiter plates (Coming Costar, Kennebunk, Me.). After 24 h of incubation in a humidified incubator at 37C., 5% CO₂ stimulation of human PBL was tested by the addition of 0.5 μCi/well ^{[3]}H-thymidine to the culture media. Cells were harvested after overnight incubation and incorporation of radioactivity was measured.

To study the MUC1 related inhibition on PHA stimulated PBL (Agrawal et al., Nat. Med., 4: 43-9 (1998)), PHA (10 μl/100 μl) was added to 100,000 freshly prepared PBL from healthy donors/100 μl RPMI, 10% FCS/well in round bottomed microtiter plates. To test inhibition of T cell stimulation by MUC1, 25 μg/ml MUC1-100 mer peptide was added. To test the reversal by IL-2 of this inhibition, 60 U/ml IL-2 or bivPH1-IL-2 was added. The MUC1-specific MAb 1G5 was used as a positive control. Cells were incubated for 6 days at 37° C., 5% CO₂ in a humidified incubator followed by ³H-thymidine labeling, harvesting and counting of the cells as described above.

Cytotoxicity Assay

The cytotoxic activity of PBL as effector cells towards the MUC1 expressing target population, ovarian carcinoma cell line OVCAR-3, was measured by ⁵¹Cr-release assay. Target cells were preincubated in PBS alone or in PBS with 5 μg/ml bivPH1 or bivPH1-IL-2 30 minutes prior to the 60 minute incubation with 1 mCi/ml/10⁶ cells ⁵¹Cr at 37C. Incubation volumes were 100 μl. Target cells were washed 3 times and resuspended in RPMI, 10% FCS at 5000 cells/50 μl and seeded into a flat bottom microtiter plate. PBL (50 μl) were added at a target (5000 cells/50 μl/well) to effector ratio (T/E) of 1:100, 1:50, 1:25 and 1:12.5. Maximum release was determined by the addition of Tween-20 to the target cells. For measurement of minimal release, no PBL were added to the target cells. To measure the influence of IL-2, 100 U/ml IL-2 was added to the appropriate wells. After overnight -incubation, cells were harvested with a supernatant harvesting system and the released ⁵¹Cr was counted in a γ scintillation counter. Percent (%) of lysis was measured as 100×(cpm test ⁵¹Cr released−cpm minimal ⁵¹Cr released/cpm maximal ⁵¹Cr released−cpm minimal ⁵¹Cr released). Tests were performed in triplicates and repeated at least three times.

IL-2 Activity Retained in BivPH1-IL-2 Immunocytokine

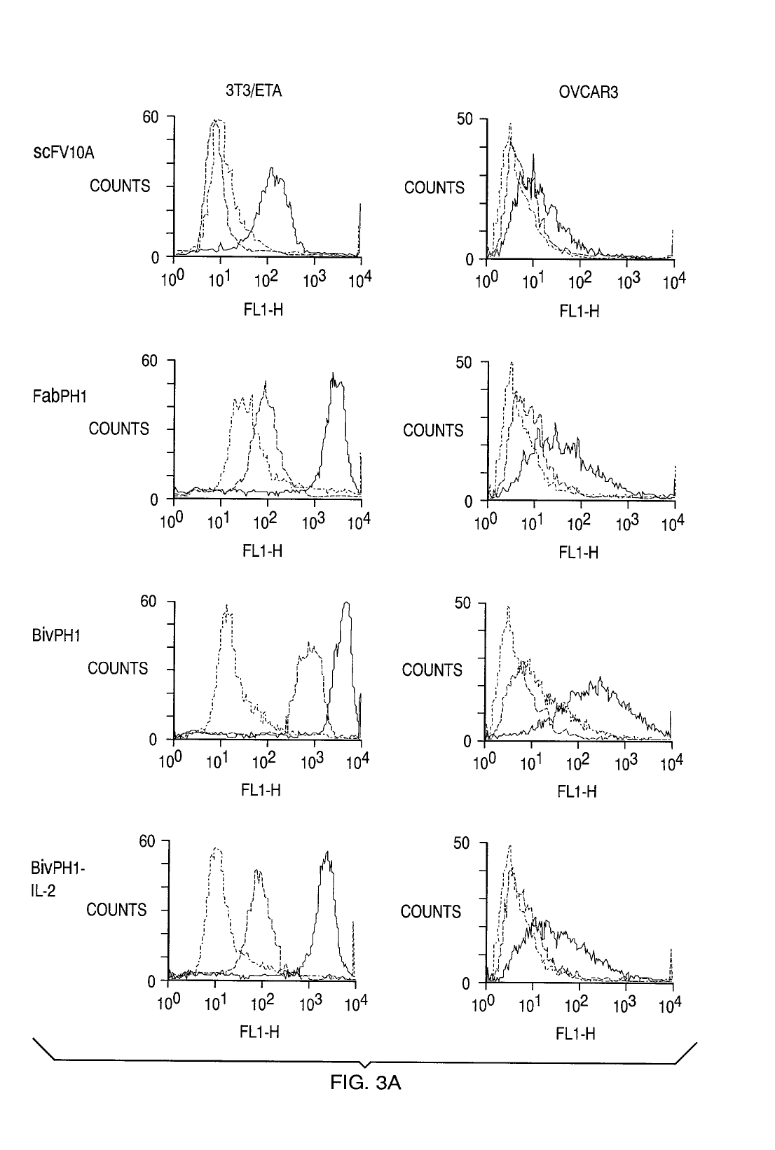

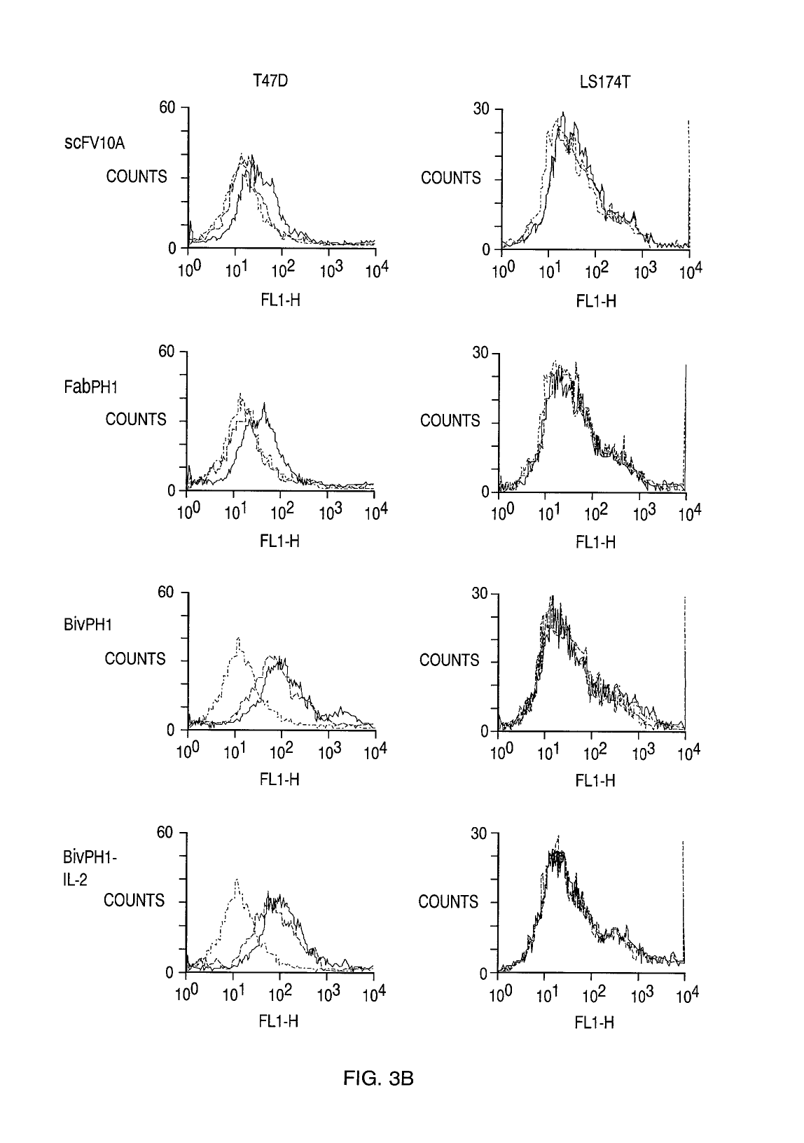

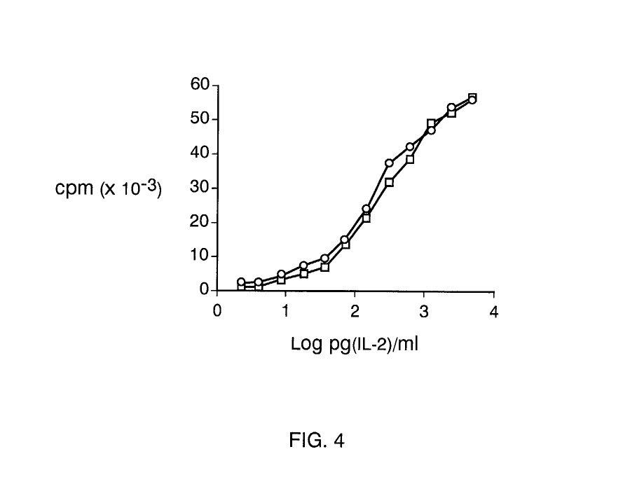

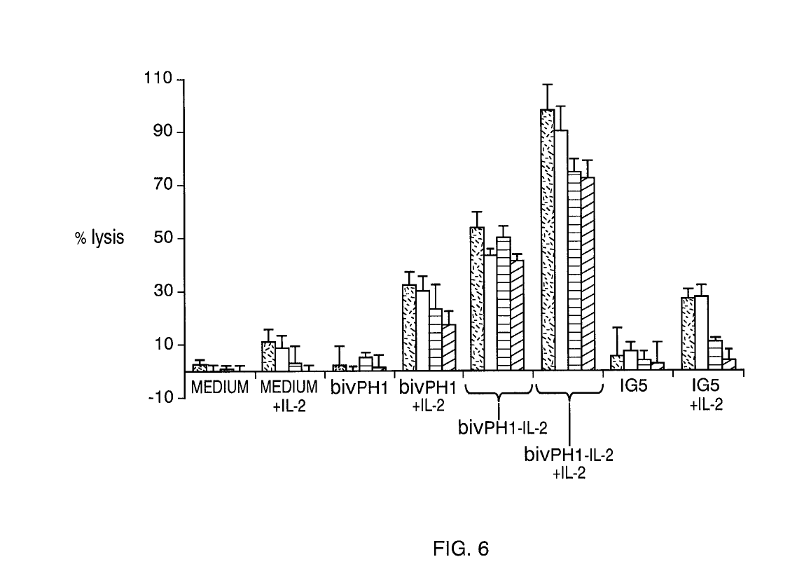

The gene cassette encoding the bivalent antibody was fused to the human IL-2 gene. The fusion protein (bivPH1-IL-2) had retained the binding characteristics in BIAcore as bivPH1 and flow cytometry (FIGS. 3A and 3B) and showed the same binding pattern in immunohistochemistry. In flow cytometry, bivPH1-IL-2 was not competed off with the MUC1 60-mer peptide although a lower concentration of bivPH1-IL-2 was used than for the other antibodies (FIGS. 3A and 3B). Comparison of bivPH1-IL-2 to rIL-2 showed that the immunocytokine has the same specific activity as commercially available rIL-2 (FIG. 4), the diabody bivPH1 did not stimulate this IL-2 dependent cell line (data not shown). This is in accordance to the results observed by others studying similar immunocytokines (Melani et al., Cancer Res., 58: 4146-54 (1998), Gillies et al., Proc. Natl. Acad. Sci. USA, 89: 1428-32 (1992)). The bivPH1-IL-2 stimulated human PBL proliferation to the same extend as native rIL-2 (FIG. 5). In an attempt to reverse MUC1-related inhibition of stimulated PBL by IL-2 as described (Agrawal et al., Nat. Med., 4: 43-9 (1998)), we added the MUC1 100-mer together with PHA and IL-2 to PBL. No inhibition of the stimulated lymphocytes by MUC1 was detected. It was possible to kill tumor cells by resting PBL when target cells were coated with bivPH1-IL-2 (FIG. 6). Moreover, upon the addition of IL-2 to the cultures, bivPH1-IL-2 as well as bivPH1 coated target cells were affected.

It has previously been shown that the principal cause of antibody-IL-2 fusion protein (IgG-IL-2) mediated killing by resting PBL in vitro is due to the induction of NK activity by interaction of FcγRIII on NK cells with the constant region of the antibodies (Naramura et al., Immunol Lett., 39: 91-9 (1994), Gillies et al., Cancer Res., 59: 2159-66 (1999)). However, this cannot be the explanation of the enhanced killing observed in these experiments since no Fc region is present on neither bivPH1 nor bivPH1-IL-2. The data suggest that the killing ability is influenced by several modes of action. First, the immunocytokine brings T cells in close proximity to tumor cells through interaction of the immunocytokine with both the IL-2 receptor and MUC1. Secondly, the MUC1 antibody covers Epotential inhibiting epitopes on the cellular MUC1 and thereby prevents inhibition of T cells. And thirdly, the IL-2 part of the immunocytokine rescues T cells from anergy. This direct killing of tumor cells mediated by resting PBL is influenced by antibody binding to the cells, which is obviously not caused by antibody dependent cell-mediated cytotoxicity (ADCC) through the Fc receptor on NK cells.

Selection and Characterization of Human Anti-MUC1 Antibodies (MUC1-specific Binding Members) from Large Non-immunized scFv and Fab Phase Libraries

As a starting point, a fully human anti-MUC1 antibody was selected from a large non-immunized human Fab library using phage display technology (de Haard et al., J. Biol. Chem., 274: 18218-18230 (1999)). Since the efficiency of immunocytokines improves when repetitive injections are administered (Melani et al., Cancer Res., 58: 4146-54 (1998)), it is important to use components with a minimal immunogenicity for the immunocytokine. The use of human antibody phage libraries allows the retrieval of human anti-MUC1 antibodies (Henderikx et al., Cancer Res., 58: 4324-32 (1998), Griffiths et al., EMBO J., 12: 725-734 (1993)), and permits design and engineering of the antibody format (size, affinity or avidity, multivalency, clearance, etc.) and effector functions for the chosen application (de Haard et al., Adv. Drug Del. Rev., 31: 5-31 (1998), Hoogenboom, Trends in Biotechnol., 15: 62-70 (1997)). To obtain an adenocarcinoma specific, high affinity/avidity antibody binding to MUC1 present on cells, a very large, non-immunized (naive) Fab library was used, containing 3.7×10¹⁰ different antibodies, on a MUC1-transfected cell line (3T3-MUC1). These cell selections were compared with previously published selections on biotinylated synthetic MUC1 peptide with the same library (de Haard et al., J. Biol. Chem., 274: 18218-18230 (1999)) and with a large scFv library with 6×10⁹ different scFv (Henderikx et al., Cancer Res., 58: 4324-32 (1998), Vaughan et al., Nature Biotechnol., 14: 309-314 (1996)) (Table 1). When selections were run using an ELISA system with coated MUC1 100-mer peptide, antibodies were only recovered from the scFv library. In contrast, selections were successful with both the scFv and Fab libraries when a biotinylated antigen was used and selection was carried out in solution.

The antibodies that were isolated from the scFv library have been described previously (Henderikx et al., Cancer Res., 58: 4324-32 (1998)): briefly, 5 different antibodies were found, with scFv 10A and 10B exhibiting the highest ELISA signal, and binding specifically to adenocarcinoma tissue; both have a relative quick off-rate (best k_{off}: 10⁻² s⁻¹) in BIAcore. In terms of number of different antibodies selected and the best off-rate, the Fab library was superior: 14 different antibodies were found, with the best off-rate in the 10⁻⁴ s⁻¹ range. Nevertheless, none of the obtained Fabs bound to cells in flow cytometry. Most likely, the flexible peptide displays selection-dominant epitopes (Hoogenboom et al., Eur. J. Biochem., 260: 774-84 (1999)) that drive the selection away from less abundant, possibly conformational epitopes on MUC1, which are present on the cell surface. When MUC1 expressing cells were used for selections, even after depletion with MUC1 negative cells, no MUC1-peptide specific Fab antibodies were found. When using similar conditions with the scFv library, no MUC1 specific antibodies were detected. Furthermore, using a combination of selections on, first, coated MUC1 100-mer, followed by panning on the MUC1-expressing cell line T47D with the scFv library, no new MUC1 specific antibodies were obtained, nor were the scFv cell binding antibodies 10A and 10B, which are nevertheless known to be present in the library, obtained.

Therefore, the selection strategy was reversed: the first two rounds were carried out on MUC1-transfected 3T3 cells, after an initial depletion step on non-transfected 3T3 cells, and rounds 3 to 5 were performed using coated MUC1 60-mer. After the 4^{th} selection round with the Fab library, 6 different antibodies, based on the BstNI fingerprint pattern, were identified with one pattern dominating the population (58%, represented by clone PH1). In the 5^{th} round of selection, 92% of the ELISA positive clones had the PH1-clone pattern. In flow cytometry of the representative clones of each of the six Fab-DNA fingerprints, only Fab PH1 bound to ETA MUC1-expressing cells.

By BIAcore analysis, human Fab antibody PH1 was shown to have a slower off-rate than any of the antibodies retrieved from the scFv library (k_{off}: 10⁻¹ s⁻¹) and was, therefore, further characterized. By indirect epitope fingerprinting (Henderikx et al., Cancer Res., 58: 4324-32 (1998)), the minimal binding epitope was determined to be the tripeptide Pro Ala Pro of the MUC1 protein core (data not shown). By DNA sequence analysis, the V_{H} of the PH1 human Fab antibody was found to be derived from the germ line segment DP47, and the V_{L} was found to be derived from the germ line sequence DPK15, both with a small number of amino acid mutations (see, Table 2). The nucleotide and corresponding amino acid sequences for the V_{H} region from PH1 are shown in SEQ ID NOS:4 and 3, respectively. The nucleotide and corresponding amino acid sequences for the V_{L} region of PH1 are shown in SEQ ID NOS:2 and 1, respectively. The sequence data revealed the framework (FR) and CDR sequences of the PH1 V_{H} and V_{L} regions (see, e.g., Table 2). In addition, these sequences are not related to the sequences of other anti-MUC1 antibodies cloned by this laboratory (de Haard et al., J. Biol. Chem., 274: 18218-18230 (1999), Henderikx et al., Cancer Res., 58: 4324-32 (1998)) or by others (Griffiths et al., EMBO J, 12: 725-734 (1993)).

Construction, Expression and Biochemical Analysis of Bivalent Anti-MUC1 Diabody and Immunocytokine Molecules

With the selected PH1 Fab antibody V genes, a fully human immunocytokine of the general formula (V_{H}-L-V_{L})-IL-2 was constructed, in which the PH1 V_{H} and V_{L} regions are covalently linked to one another via a linker peptide L, and the V_{H}-L-V_{L} moiety is covalently linked at its carboxy terminal amino acid to the amino terminal amino acid residue of an IL-2 protein. The desired anti-MUC1 immunocytokine molecule was designed to have several particularly advantageous properties: (1) to be larger than the 45 kD scFv-IL-2 molecular weight, (i.e., above the renal filtration threshold) for obtaining a longer circulation half-life, (2) to possess an avidity advantage in its binding to MUC1, by having two distinct binding sites on the same molecule, which, unlike the monovalent PH1 Fab antibody, fully exploits the multimeric nature of the MUC1 antigen, and (3) to not have an Fc receptor binding domain (i.e., CH2 and CH3 domains of IgG not present), which was recently shown to interfere negatively with the efficacy of antibody-IL-2 fusion products (Gillies et al., Cancer Res., 59: 2159-66 (1999)). Such properties were attained by constructing a bivalent diabody-IL-2 fusion of 90 kD molecular weight (see, FIG. 1). The V genes were reformatted in the diabody V_{H}-linker-V_{L} format (Holliger et al., Proc. Natl. Acad. Sci. USA., 90: 6444-8 (1993)). The short, 5 amino acid residue linker (L1) drives the preferential formation of diabodies, i.e., two single-chain Fv molecules that are paired non-covalently to form a dimer with two functional binding sites. The bivalent diabody gene cassette was subsequently fused to the human IL-2 gene. The bivPH1 diabody and the bivPH1-IL-2 diabody immunocytokine fusion proteins were both expressed in E. coli, and both fusion proteins were purified from the periplasmic extract using immobilized metal affinity chromatography (IMAC).

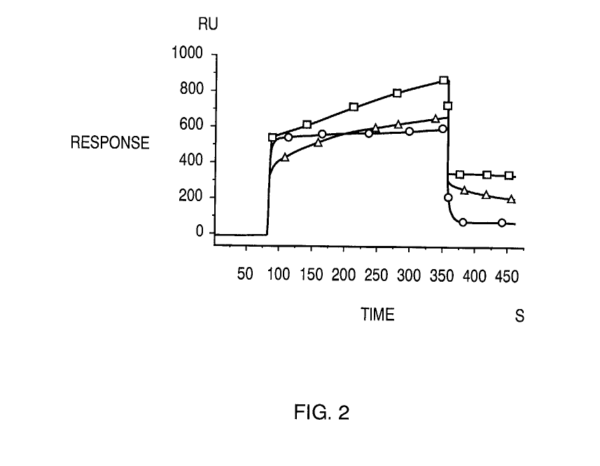

The binding characteristics of the Fab PH1 and scFv 10A antibodies were compared with the two diabody constructs, i.e., the bivalent bivPH1 diabody and the bivalent bivPH1-IL-2 immunocytokine fusion in BIAcore (FIG. 2). The bivalent diabodies bound with off-rates at least 10 times stronger as compared to the Fab binding (k_{off}: 10⁻³ s⁻¹). These binding characteristics were measured on synthetic MUC1 80-mer peptide chips (with 90 RU immobilized antigen). The relative off-rate of the bivalent diabody molecules measured under these optimal conditions was below 10⁻⁴ s⁻¹. This relative off-rate was dependent on the conditions of measurement, such as antigen-density on the chip. The 20 amino acid peptide of MUC1 was repeated 30 to 100 times on cells, in a variable number of tandem repeats (Swallow et al., Nature, 328: 82-4 (1987)). The avidity effect of the bivalent bivPH1 antibody on cells was expected to be at the least of the same magnitude due to binding and rebinding effects on the same molecule. Hence, the binding effect of the monovalent versus bivalent antibodies was measured on cells in flow cytometry (FIGS. 3A and 3B). The bivPH1 diabody bound considerably better to the MUC1-transfected 3T3 cell line, the ovarian carcinoma cell line OVCAR-3, and the breast cancer cell line T47D, than the scFv 10A and the PH1 Fab antibodies, although the same amounts of scFv, PH1 and bivPH1 were used. This binding was one log higher when bivPH1 was compared to scFv 10 A and about 0.5 log better when compared to Fab PH1. This stronger binding to cells was confirmed by preincubation of the antibodies with the MUC1 60-mer where the inhibition of antibody cell binding by the MUC1 60-mer was complete in the case of the scFv 10A antibody, almost complete in the case of the PH1 Fab antibody, and partial in the case of the bivPH1 diabody. This partial inhibition was not due to non-specific binding since none of the antibodies bound to the non-transfected murine fibroblast cell line 3T3 nor to the highly glycosylated colon cell line LS147T. Inhibition by the MUC1 60-mer peptide was less pronounced in the case of the T47D cell line than in the case of the OVCAR-3 cell line.

Effector Function of the Bivalent Human Immunocytokine BivPH1-IL-2

Because of the relative short distance between the two MUC1 binding regions and the IL-2, it was important to test whether this fusion format would impair the IL-2 activity. Therefore, an IL-2 dependent murine T cell line (CTLL-16) was stimulated with increasing amounts of bivPH1-IL-2 and the stimulatory efficiency was compared with that of commercial available recombinant IL-2 (rIL-2). As shown in FIG. 4, both rIL-2 and bivPH1-IL-2, stimulated the murine T cell line with an equal activity, while bivPH1 did not stimulate (data not shown); similarly, rIL-2 and bivPH1-IL-2 stimulated PBL equally well (FIG. 5). In an attempt to verify whether IL-2 and bivPH1-IL-2, were able to reverse the MUC1-related inhibition of T cells, PBL were incubated with PHA and MUC1 for 6 days and tried to reverse the inhibition. However, no inhibition of T cell activation by MUC1 was observed so that reversal of inhibition could not be studied using this protocol.

To prove the fumctionality of both sites of the immunocytokine, a ⁵¹Cr-release assay was performed (FIG. 6). The MUC1 expressing target cells OVCAR-3 were preincubated with bivPH1 or bivPH1-IL-2 and washed. Resting PBL did not mediate lysis of the target cells and the addition of 100 U/ml rIL-2 was not efficient in improving the lysis. The bivPH1 diabody did not significantly increase the level of lysis, though with the addition of rIL-2, lysis rose considerably above the background level (p<0.05). The bivPH1-IL-2 immunocytokine fuision protein enhanced the lysis of target cells by resting PBL more than the non-fusion combination bivPH1 and rIL-2 (p<0.03), proving that the MUC1 binding site as well as the effector site is functional (see, FIG. 6). Moreover, with the addition of rIL-2 to the immunocytokine coated target cells, complete killing was achieved (p<0.001). No killing was observed when the colon cell line LS174T, not binding PH1 in flow cytometry (FIG. 3B), was used as a target in a similar assay (data not shown).

Half-life of Dissociation of BivPH-1-IL-2 Immunocytokine

The PH1 Fab antibody was chosen as the source of V_{H} and V_{L} regions to construct an immunocytokine because of the PH1 cell binding properties in flow cytometry, adenocarcinoma associated immunohistological staining pattern, and the slowest off-rate of all the clones tested. For antibody-mediated immunotherapy, increasing evidence has accumulated that high affinity of the antibody is important for antibody-mediated killing (Velders et al., Br. J. Cancer, 78: 478-83 (1998)); similarly, increased binding due to avidity can benefit tumor uptake of recombinant antibody fragments (Adams et al., Cancer Res., 53: 4026-34 (1993)). The off-rate of the monovalent PH1 Fab on coated 80-mer in BIAcore was 10⁻³ s-1, which indicates that a similarly monovalent effector molecule would have a half-life of dissociation from the antigen of 11 minutes. Therefore, an improvement of binding strength was desirable. Since MUC1 has a variable number of tandem repeats, the goals were: (1) to improve the avidity by making a bivalent form of the PH1 Fab (bivPH1) and (2) to obtain the dissociation effect as described for multivalent receptors (Goldstein et al., Immunol. Today, 17: 77-80 (1996)). Indeed, in BIAcore, the bivPH1 diabody antibody molecule had a more than 10 times slower off-rate: the half-life of binding improves on this antigen surface from about 11 minutes to 2 hours (see, FIG. 2). The bivalency effect of the bivPH1 diabody antibody molecule described herein was similarly dramatic on cells that express a VNTR of MUC1 when measured by flow cytometry (see, FIG.3). Binding intensity increased by approximately 1 log compared with the scFv 10A and 0.5 log compared with the PH1 Fab antibody molecule. Moreover, this binding was not easily competed off by 100 μg/ml of the MUC1 60-mer peptide, confirming the importance of the number of repeats in the MUC1 molecule for the retention binding.

The kinetics of dissociation of antibodies from multivalent receptors expressed on the cell surface such as MUC1, has been studied extensively. If no rebinding occurs, the half-life of dissociation of the complex, described by the formula t_{½}≈1/k_{off}(1nN−1n(1n2)+1n2/2N), increases with the valency of the antigen (N) (Goldstein et al., Immunol. Today, 17: 77-80 (1996)). The t_{½} (half-life of dissociation) for bivPH1-IL-2 immunocytokine on cellular MUC1 can be calculated using this formula and the value of k_{off} measured on BIAcore. Presuming the MUC1 glycoprotein has 100 tandem repeats, this would result in an estimated half-life for dissociation of 14 hours. Furthermore, the rebinding of the antibodies is additionally affected by the density of the antigen (MUC1) on the cell surface (Goldstein et al., Biophys. J., 56: 955-66 (1989)), which is overexpressed in a variety of adenocarcinomas (Burchell et al., Cancer Res.,47: 5476-5482 (1987)). Accordingly, the tumor dissociation half-life of the bivPH1-IL-2 immunocytokine on cells will be substantially higher than 2 hour.

In conclusion, the bivPH1-IL-2 not only directs IL-2 to the tumor site and activates T cells, but also covers potentially inhibitory epitopes, which are desired properties for improving tumor cell killing and further preventing anergy of stimulated T cells in cancers, such as adenocarcinoma.

### Example 2

Affinity Maturation of Human MUC1-Specific Monovalent PH1 Fab Antibody

This example demonstrates the use of phage display methodology to carry out an in vitro selection (i.e., affinity maturation) for Fab antibodies containing monovalent binding sites with an enhanced affinity for MUC1 from libraries of mutated heavy chain molecules from the PH1 Fab antibody described above. Mutagenesis was directed toward residues in the heavy chain CDR1 and CDR2 regions that are frequently mutated in vivo (known as “hot spots” of in vivo mutagenesis), and toward the complete heavy chain CDR3 region.

Escherichia coli (E. coli) TG1: K12, D(lac-pro), supE, thi, hsdD5/F′ traD36, proA⁺B⁺, lac.I^{q}, lacZDM15 was used as the host in the phage display affinity selection procedure.

Preparation of V_{H} Libraries

(a) CDR3 Libraries

The V_{L}C_{L} of PH1 was cloned as an ApaLI-AscI fragment in the phagemid pCES1 vector (de Haard et al., 1999), to yield pCES-PH1-VL. The V_{H} of PH1 was amplified using primers #206 and one of the mutagenic CDR3 primers, as indicated below (see, Table 3). The PCR products were cloned as an SfiI-BstEII fragment in pCES-PH1-VL.

(b) Hotspot Library

In a first PCR, the CDR1 and the CDR2 libraries, were prepared with the PH1-VH as template using the primer pair #701 and #87 and primer pair #206 and #702, respectively (see Table 3). The DNA encoding these libraries were combined by a PCR assembly reaction using primers #206 and #87 and the resulting VH-genes cloned as a SfiI-BstEII fragment in pCES-PH1-VL for phage display and selection.

TABLE 3
Oligonucleotides Used in Affinity Maturation of MUC1 Binding Domain of PH1

| | |
|---|---|
| (a) Primers used for introduction of mutations | |
| #701 Hotspot CDR1 oligo | |
| 5′ -GGA TTC ACG TTT AGA A*G*T* AAC GCC ATG GGC TGG-3′ | (SEQ ID NO:33) |
| #702 Hotspot CDR2 oligo | |
| 5′ -CAC GGA GTC TGC GTA G*T*A* TGT G*C*T* GCC ACC ACT ACC ACT-3′ | (SEQ ID NO:34) |
| CDR3 spiked oligo | |
| 5′ -CTA TGA GAC GGT GAC CAG GGT TCC CTG GCC CCA G*T*A* G*T*C* A*A*T* | (SEQ ID NO:35) |
| G*G*G* G*T*C* C*C*A* A*A*C* G*C*C* C*C*C* C*C*C* G*G*T* A*T*G* T*T*T* | |
| C*G*C* ACA ATA ATA TAC GGC-3′ | |
| CDR3 random oligo 1 | |
| 5′ -CTA TGA GAC GGT GAC CAG GGT TCC CTG GCC CCA GTA GTC AAT GGG GTC | (SEQ ID NO 36) |
| CCA AAC MNN MNN MNN MNN MNN TTT CGC ACA ATA ATA TAC GGC-3′ | |
| CDR3 random oligo 2 | |
| 5′ -CTA TGA GAC GGT GAC CAG GGT TCC CTG GCC CCA GTA GTC MNN MNN MNN | (SEQ ID NO: 37) |
| MNN MNN GCC CCC CCC GGT ATG TTT CGC ACA ATA ATA TAC GGC-3′ | |
| Asterisked nucleotides indicate the following mixtures: | |
| A*=90%A + 2.5%A + 2.5%C + 2.5%G + 2.5%T | |
| C*=90%C + 2.5%A + 2.5%C + 2.5%G + 2.5%T | |
| G*=90%G + 2.5%A + 2.5%C + 2.5%G + 2.5%T | |
| T*=90%T + 2.5%A + 2.5%C + 2.5%G + 2.5%T | |
| (b) Primers used for amplification of V_{H} of PH1 Fab antibody | |
| #87, HuJH4-5-FOR | |
| 5′ -TGA GGA GAC GGT GAC CAG GGT TCC-3′ | (SEQ ID NO:38) |
| #206, VH1c back Sfi | |
| 5′ -GTC CTC GCA ACT GCG GCC CAG CCG GCC ATG GCC SAG GTC CAG CTG GTR | (SEQ ID NO:39) |
| CAG TCT GG-3′ | |
| Nucleotide ambiguity codes: | |
| M =A or C; R =A or G; S =C or G; N =A, C, G or T | |

underlined sequences indicate encoded restriction sites

Affinity Selection

(a) Selection on Biotinylated MUC1

Selections were performed on biotinylated MUC1 60-mer as described by Hawkins et al. (J. Mol. Biol., 226: 889-96 (1992)) with some modifications: phage were incubated on a rotator wheel for 1 hour in 2% M-PBST (PBS supplied with 2% skimmed milk powder and 0.1% Tween-20). Meanwhile, 100 μl of streptavidin-conjugated paramagnetic beads (Dynal, Oslo, Norway) were incubated on a rotator wheel for 2 hour in 2% M-PBST. Biotinylated antigen was added to the pre-incubated phage and the mixture was incubated on a rotator wheel for 30 minutes. Next, the streptavidin-beads were added and the mixture was left on the rotator wheel for 15 minutes. After 14 washes with 2% M-PBST and one wash with PBS, phage particles were eluted with 950 μl 0.1 M triethylamine for 5 minutes. The eluate was neutralized by the addition of 0.5 ml Tris-HCl (pH 7.5) and was used for infection of log-phase E. coli TG1 cells. The TG1 cells were infected for 30 minutes at 37° C. and were plated on 2×TY (16 g Bacto-trypton, 10 g yeast extract and 5 g NaCl per liter) agar plates, containing 2% glucose and 100 μg/ml ampicillin. After overnight incubation at 30° C., the colonies were scraped from the plates and used for phage rescue as described (Marks et al., J. Mol. Biol. 222, 581-597 (1991)).

(b) Selection on MUC1-expressing Cells

Alternating selections were performed on the T47D breast cancer cell line (Hanisch et al., 1996) and on the OVCAR-3 ovarian carcinoma cell line, both are known to express tumor-associated glycoforms of MUC1. Briefly, 10¹² phage and cells (10⁷ T47D, 5×10⁶ OVCAR, 2×10⁶ T47D and 2×10⁶ OVCAR for rounds 1, 2, 3 and 4, respectively) were preincubated with 2% M-PBS (PBS supplied with 2% skimmed milk powder) for 30 minutes; then phages were added to the cells. After 1 hour of incubation, cells were washed 10 times with M-PBS+10% FCS. Specific phage were eluted and used for infection of exponentially growing TG1 cells as described earlier.

ELISA and Kinetic Measurement Using Surface Plasmon Resonance in BIAcore

Soluble Fabs were produced as described (Roovers et al., Br. J. Cancer, 78: 1407-16 (1998)). ELISAs were performed as described by Henderikx et al. (Cancer Res., 58: 4324-4332 (1998)), except the biotinylated MUC1 60-mer was used. The selected PH1 and the affinity-matured antibodies were evaluated for affinity by surface plasmon resonance (SPR) on a BIAcore 2000 apparatus (BIAcore AB, Uppsala, Sweden). Channels of a biotin chip were coated with a MUC1 15-mer, containing the minimal PH1 epitope, PAP, (Ac-PDTRPAPGSTAPPAL-NH₂, (SEQ ID NO:40) 50 RU or 320 RU) or a 60-mer (NH₂-(VTSAPDTRPAPGSTAPPAHG)₃-COOH (i.e., containing three copies of SEQ ID NO:8 (von Mensdorff-Pouilly et al., Tumor Biol., 19: 186-195 (1998), 50 RU) in HBS-EP buffer (Pharmacia). One surface was blocked with biotin (15 RU) and used as a negative control. The antibodies were injected in HBS-EP buffer. To minimize rebinding of the antigen binding fragments, the flow speed was 30 μl/sec. Affinity calculation was performed with the BIA-evaluation software provided by the BIAcore. The affinities of the Fabs were calculated according to a 1:1 stoichiometry at steady state.

DNA Sequencing

The nucleotide sequences of the selected Fabs were determined using dideoxy sequencing. Products of the sequencing reaction were analyzed on a semi-automated sequencer (Alf Express; Pharrnacia). The oligonucleotide used for VH sequencing was CHiFOR: 5′-GTC CTT GAC CAG GCA GCC CAG GGC-3′ (SEQ ID NO:9).

FACS Analysis

Specific binding of the Fabs was measured by FACScalibur analysis (Becton Dickinson, Oxnard, Calif.) as described by Henderikx et al. (Cancer Res., 58: 4324-4332 (1998)). For affinity studies on cells with recombinant Fabs, the following flow cytometry experiment was carried out. Fab fragments were purified from the periplasmic fraction by IMAC and gel filtration as described in (Roovers et al., Br. J. Cancer, 78: 1407-1416 (1998)). Protein concentrations were measured with the bicinchoninic acid method (Sigma, St. Louis, Mo., USA). Two-fold serial dilutions of these Fabs were made and incubated, for each dilution point, with 2×10⁵/100 μl ETA cells (transfected 3T3 cells (Acres et al., J. Immunol., 14: 136-1443 (1993)). After trypsinisation, cells were washed one time in RPMI 10% FCS, 0.1% NaN₃ (incubation buffer). Then, cells were incubated with appropriate dilution for 1 hour at room temperature (RT), on a rotator. As negative controls, 2×10⁵ 3T3 mouse fibroblast cells were also incubated with the highest concentrations of antibodies. As a secondary negative control, ETA cells without primary antibody were used. Cells were spun down by centrifugation for 3 minutes at 611×g. Between incubations, cells were washed with incubation buffer. In a second incubation, anti-Myc antibody (6 μg/ml 9E10), directed against the Myc-tagged Fabs, was added for 30 minutes in incubation buffer at RT. After washing once, rabbit anti-mouse-FITC was used at RT for 30 minutes (Dako). Detection of bound antibodies was performed by means of flow cytometry on a FACSCalibur (Becton Dickinson, Oxnard), and results analyzed with the CELLQuest program (Becton Dickinson). Mean intensity was plotted against the concentration of the antibodies.

Results and Analysis

The affinity maturation selection procedure employed in this study involved mutagenesis to the variable region of the heavy chain of the PH1 Fab antibody, and within this VH to two types of residues: (1) the residues which frequently confer a higher affinity to the antibody-antigen interaction in vivo (“hot spots”): residue 31 in VH-CDR1 and residues 56 and 5, in the VH-CDR2; and (2) the CDR3 regions, which sits at the heart of the antigen combining site, and mutagenesis of which frequently results in higher affinity antibodies (Hoogenboom, Trends Biotechnol., 15: 62-10 (1997)).

Specifically, four different libraries were assembled: one CDR1-2 hot spot library (HSPOT), with mutations at amino acid positions 31, 57, 59 of SEQ ID NO:3; and three libraries for the heavy chain CDR3 (H-CDR3). The HSPOT library was made by assembly-PCR of two DNA fragments, one with the CDR1 region harboring a spiked residue 31, the other with a CDR2 region with residues 57 and 59 spiked and a wild-type CDR3, and cloning this VH gene for expression with the PH1 light chain as Fab fragments displayed on phage (see, HSPOT CDR1 and HSPOT CDR2 oligonucleotides in Table 3). Since the H-CDR3 has a length of 12 amino acid residues, the theoretical diversity in this region is 20¹².

Two different RAN1 and RAN2 libraries were made, with only 5 amino acid residues in each library completely randomized (see, CDR3 random oligonucleotides 1 and 2 in Table 3). The theoretical diversity of these individual libraries would therefore be 3.3×10⁶, represented by 3.3×10⁷ variants in a library with 32 possibilities per codon.

To access additional diversity in the neighboring residues of the CDR3, in the H3 region at amino acid residues 97 and 98 of SEQ ID NO:3, as well as in the last two joining-region encoded residues of the CDR3, amino acid residues 109 and 110 of SEQ ID NO:3, a library called SPIKE was made in which oligonucleotides (spiked at a level of 7.5% of mutant nucleotides with 92.5% wild-type) were used to mutagenize a region of 14 residues. The CDR3-libraries were made by PCR with mutant oligonucleotides (see, CDR3 spiked oligo in Table 3) of the VH of PH1 Fab antibody and cloning of the resulting DNA as an SfiI-BstEII fragment into pCES1-PH1-VL.

All actual library sizes were over 10⁸ clones (see, Table 4).

TABLE 4
Libraries

| | V_{H} Region | | | | | |
|---|---|---|---|---|---|---|
| | FR3 | CDR3 | FR4 | | NT pattern/mut. freq. | % positive clones |
| Library | CAK | HTGGGVWDPIDY | WG | Size | per mutant clone | in phage ELISA |
| RAN1 | | ***** | | 1.8 × 10⁸ | NN (T/G) | 1/20 |
| RAN2 | | ***** | | 2.0 × 10⁸ | NN (T/G) | 3/20 |
| SPIKE | ** | ************ | | 2.1 × 10⁸ | 4/42 | 8/20 |
| HSPOT | | (wt) | | 3.1 × 10⁸ | 2/9 | 17/20 |

*= mutagenized, (wt) = wild type (PH1) sequence

Clones from the unselected libraries were analyzed by sequencing to confirm the mutagenesis pattern, and by ELISA to test for binding to the MUC1 antigen. Not surprisingly, a high frequency of the clones of the HSPOT library were positive as phage antibody for MUC1 binding: most are indeed wild type in sequence (data not shown), and this library has only 8000 variants spread over three residues. Similarly the SPIKE library yields a high frequency of antigen binding variants of PH1, here though with 2-3 amino acid alterations per clone (see, Table 5). It was more striking to find many ELISA positives (detectable signal at OD₄₅₀) in the unselected RAN1 and RAN2 libraries, where a complete stretch of the CDR3 is altered (Table 5). It should be kept in mind that the use of phage particles which can display multiple antibodies per particle, promotes avid binding in this ELISA, and affinity differences between clones are readily masked.

TABLE 5
Sequences of unselected clones

```
                                                                    V_{H} Domains
              --FR3-- --------------------CDR3--------------------- --FR4--
Clone
PH1 A K H T G G G V W D P I D Y W G (97-112 of SEQ ID NO:3)
              gcg aaa cat acc ggg ggg ggc gtt tgg gac ccc att gac tac tgg ggc (289-336 of SEQ ID NO:4)
ELISA OD₄₅₀=0.692
RAN1 library
2 A K H N T S K V W D P I D Y W G (SEQ ID NO:41)
            gcg aaa cat aat acg tct aag gtt tgg gac ccc att gac tac tgg ggc (SEQ ID NO:42)
ELISA OD₄₅₀=0.115
3 A K S S T T T V W D P I D Y W G (SEQ ID NO:43)
            gcg aaa tct agt act acg acg gtt tgg gac ccc att gac tac tgg ggc (SEQ ID NO:44)
ELISA OD₄₅₀=0.138
5 A K & P M A N V W D P I D Y W G (SEQ ID NO:45)
            gcg aaa tag cct atg gcg aat gtt tgg gac ccc att gac tac tgg ggc (SEQ ID NO:46)
ELISA OD₄₅₀=0.361
6 A K & H T K T V W D P I D Y W G (SEQ ID NO:47)
            gcg aaa tag cat acg aag acg gtt tgg gac ccc att gac tac tgg ggc (SEQ ID NO:48)
ELISA OD₄₅₀=0.097
8 A K I T V S R V W D P I D Y W G (SEQ ID NO:49)
            gcg aaa att act gtt agt cgt gtt tgg gac ccc att gac tac tgg ggc (SEQ ID NO:50)
ELISA OD₄₅₀=0.238
B9 A K R Y L Y D V W D P I D Y W G (SEQ ID NO:51)
            gcg aaa cgt tat ctg tat gat gtt tgg gac ccc att gac tac tgg ggc (SEQ ID NO:52)
ELISA OD₄₅₀=0.236
RAN2 library
10 A K H T G G G T L Q R L D Y W G (SEQ ID NO:53)
            gcg aaa cat acc ggg ggg ggc act ttg cag cgg ctg gac tac tgg ggc (SEQ ID NO:54)
ELISA OD₄₅₀=0.103
11 A K H T G G G T Q T P C D Y W G (SEQ ID NO:55)
            gcg aaa cat acc ggg ggg ggc act cag act ccg tgt gac tac tgg ggc (SEQ ID NO:56)
ELISA OD₄₅₀=0.100
13 A K H T G G G R R I C H D Y W G (SEQ ID NO:57)
            gcg aaa cat acc ggg ggg ggc cgt cgt att tgt cat gac tac tgg ggc (SEQ ID NO:58)
ELISA OD₄₅₀=0.421
15 A K H T G G G & R & & R D Y W G (SEQ ID NO:59)
            gcg aaa cat acc ggg ggg ggc tag cgg tag tag cgg gac tac tgg ggc (SEQ ID NO:60)
ELISA OD₄₅₀=0.175
D6 A K H T G G G Q K L Q L D Y W G (SEQ ID NO:61)
            gcg aaa cat acc ggg ggg ggc cag aag ctg cag ctg gac tac tgg ggc (SEQ ID NO:62)
ELISA OD₄₅₀=0.382
SPIKE library
20 A &/S H T G G R G W D P I D Y W G (SEQ ID NO:63)
            gcg tsa cat acg ggg ggg cgc ggt tgg gac ccc att gac tac tgg ggc (SEQ ID NO:64)
ELISA OD₄₅₀=0.109
21 A N Q T G G G V W D P I D Y W G (SEQ ID NO:65)
            gcg aac cag act ggg ggg ggc gtt tgg gac ccc att gac tac tgg ggc (SEQ ID NO:66)
ELISA OD₄₅₀=0.108
22 A R H T G G G V W D P I Y Y W G (SEQ ID NO:67)
            gcg aga cat acc ggt ggg ggc gtk tgg gat ccc ata tac tac tgg ggc (SEQ ID NO:68)
ELISA OD₄₅₀=0.663
23 A K P T G G G A W D P I D Y W G (SEQ ID NO:69)
            gcg aaa cct acc ggg ggg ggc gct tgg gac ccc att gac tac tgg ggc (SEQ ID NO:70)
ELISA OD₄₅₀=0.373
25 A K H T G V G V W H P I Y Y W G (SEQ ID NO:71)
            gcg aaa cat acc ggg gtg ggc gtt tgg cac ccc atc tac tac tgg ggc (SEQ ID NO:72)
ELISA OD₄₅₀=0.315
```

mutated residues indicated in bold; nucleotide “k” indicates residue may be guanine or thymine; “&” indicates end of amino acid sequence because mutation in nucleotide sequence forms a translational stop codon (tga, taa, tag)

The three CDR3 libraries did contain a low frequency of clones with the wild-type sequence of the PH1-VH (4/21 clones with the mix of the 3 libraries; see, Table 6), most likely due to pass-through of the original pCES1-PH1-Fab used as PCR-template; provided higher affinity variants of PH1 are present in the libraries, these wild-type phage should not cause any problems in the affinity maturation process.

The bacterial stocks containing the PH1-based libraries were rescued with helper phage and phage subjected to various selection regimens. To sample these libraries as fully as possible as well as probe cellular MUC1, three different selection conditions were followed, including (a) selections on decreasing amounts of the MUC1 peptide, (b) selections using the antibody PH1 as a competitor, and (c) selections on whole cells.

Selection on MUC1 Peptide

First phage from the rescued RAN1, RAN2, and SPIKE libraries were mixed and selected on biotinylated MUC1 60-mer peptide, which contains three times the 20-mer repeat sequence of the MUC1-1 sequence. Three rounds of selection with decreasing amounts of antigen (60-mer) were performed; the data on this approach are depicted in Table 6.

TABLE 6
Selections of the CDR3-libraries (mix) on biotinylated MUC1 60-mer

| | | [60-mer] | I/O | % pos. Clones | % WT | representative |
|---|---|---|---|---|---|---|
| Round | input | (nM) | (input/output) | (Fab ELISA) | seq. clones | clones |
| Unselected | | | | 6/30 | 4/21 | |
| I | 6.3 × 10^{11†} | 10 | 1.2 × 10⁵ | 18/30 | 6/12 | 3B10 |
| II | 1.2 × 10¹² | 10 | 4.3 × 10³ | 26/30 | 8/18 | |
| | | 1 | 4.3 × 10⁴ | 9/20 | 3/7 | 3D6 |
| III | 2.9*10^{12‡} | 0.1 | 1.5 × 10⁶ | 12/30 | 2/10 | |
| | | 0.01 | 5.2 × 10⁶ | 8/30 | 1/5 | |

^{†}of each library; ^{‡}of 1 nM selection; WT = wild type

An important issue was how to determine the concentration of antigen for selection. The Fab PH1 has an affinity of 1.4 micromolar (μM) for the 60-mer peptide antigen with a very fast off-rate, yet it was selected from a naive phage antibody library. Most likely avidity caused by display of multiple Fabs on the surface of the phage particles contributed to its selection. Since the affinity constant for Fab binding to a 15-mer MUC-1 peptide with just once the epitope of the antibody, is identical to that of binding to the 60-mer (data not shown), the multivalent nature of the antigen appears to have no significant role. Prior work indicated that antigen concentrations can be 100 to 1000-fold lower than the Kd of the antibody, and selection is still possible (Schier et al., J. Mol. Biol., 263: 551-567 (1996)). Thus, the first round of selection was carried out using the 60-mer peptide at an initial concentration of 10 nM, and, thereafter, decreasing this number 10-fold in subsequent steps as indicated in Table 6. Since we do not known what the spread of affinities of clones in the library, the correct concentration can only be determined empirically.

In the first and second selection, a sharp decrease in the ratio Input/Output (I/O) of the phage titers was noted, but further selection in round 3 with less antigen showed an increase again. Similarly, the frequency of positive clones in Fab ELISA increased first to nearly 90% in round 2 when 10 nM antigen was used, and 45% when only 1 nM was used; the frequency decreased again in the third round of selection with 100 and 10 picomolar (pM). This indicated that under these conditions, many lower affinity clones failed to be selected. It was possible that under those conditions the highest affinity clones should become enriched preferentially. This was confirmed by the initial selection and later decrease of the frequency of clones with a wild-type sequence.

Competitive Selection

In a second approach we attempted to select the higher affinity variants of PH1 using competition with the wild-type PH1 Fab fragment. Libraries were now separately selected on the biotinylated MUC-1 60-mer in the presence of 0.2 or 1 μM of the PH1 Fab fragment. After 6 hours of co-incubation of phage, antigen and soluble competitor, the phage that remained bound to the biotinylated antigen were retrieved using streptavidin-coated beads. Phage titers and selection data are summarized in Table 7.

TABLE 7
Selections on biotinylated MUC1 in the presence of soluble PH1 Fab

| | [60-mer] | | | [PH1] | | % pos. Clones | | representative |
|---|---|---|---|---|---|---|---|---|
| Round | (nM) | Library | I | (μM) | I/O | (Fab ELISA) | % WT | clone |
| I | 10 | RAN1 | 6.4 × 10¹⁰ | 1 | 7.2 × 10⁴ | 1/20 | 1/1 | |
| | | | | 0.2 | 9.6 × 10⁴ | 0/20 | | |
| | | RAN2 | 5.2 × 10¹⁰ | 1 | 2.9 × 10⁴ | 1/20 | | |
| | | | | 0.2 | 4.0 × 10⁴ | 0/20 | | |
| | | SPIKE | 1.6 × 10¹¹ | 1 | 1.2 × 10⁵ | 1/20 | 0/1 | 5C8 |
| | | | | 0.2 | 7.6 × 10⁴ | 1/20 | 0/1 | |
| | | HSPOT | 6.4 × 10¹⁰ | 1 | 5.8 × 10⁴ | 6/20 | 3/3 | |
| | | | | 0.2 | 4.6 × 10⁴ | 6/20 | 5/5 | |
| II† | 1 | RAN1 | 2.6 × 10¹² | 1 | 2.3 × 10⁶ | 0/25 | | |
| | | RAN2 | 3.7 × 10¹² | 1 | 6.2 × 10⁶ | 1/25 | 0/1 | |
| | | SPIKE | 2.6 × 10¹² | 1 | 2.5 × 10⁶ | 6/25 | 1/6 | 7D1, 7F3, 7F9 |
| | | HSPOT | 3.8 × 10¹² | 1 | 1.2 × 10⁶ | 10/25 | 7/9 | |

I = input; O = output; †= outputs from the 1 μM PH1 competition selection were used

As expected, the I/O ratio decreased when compared to the selection without competition (Table 6), but more strikingly, the frequency of MUC1-positive clones dropped dramatically (from 60%, 18/30 in Table 6) for the mix to 5% (3/60, Table 7) for the individual libraries), indicating that the selection with competition worked. Similarly, the frequency of MUC-1 positives selected from the HSPOT library decreased after 1 selection round (compare frequencies in Tables 4 and 7), but these clones are still wild-type sequence, which of course dominate the unselected library. In the second selection round with only 1 nM MUC1 antigen also from the HSPOT library, clones appeared that were not wild type.

Selections on cells

The two other procedures led to the isolation of variants with up to a 3.5-fold increase of the Kd for the MUC1 peptide (see below). However, there was a possibility that there were variants in the libraries that would more strongly recognize the cellular MUC1, but show only a minor improvement of the affinity towards the peptidic MUC1 antigen. Therefore, as an alternative to the selections on MUC1 peptide, cells expressing the (partially glycosylated) form of the MUC1 antigen were used in a selection. To prevent the unlikely yet theoretically possible selection of clones for antigens other than MUC1, the selections were alternated between two cell lines, the T47D breast carcinoma and OVCAR ovarian carcinoma cell lines. The selection data are depicted in Table 8.

TABLE 8
Alternating selections on T47D and OVCAR cells

| | | | | Out- | | % positive | % |
|---|---|---|---|---|---|---|---|
| | | Cell | Input | put | | clones | wild |
| Library | Round | line | (I) | (O) | I/O | (Fab ELISA) | type |
| RAN1 | I | T47D | 3.4 × | 1.4 × | 2.4 × | 4/20 | 1/1 |
| | | | 10¹² | 10⁷ | 10⁵ | | |
| | II | OVCAR | 2.2 × | 3.5 × | 6.3 × | 0/20 | |
| | | | 10¹² | 10⁶ | 10⁵ | | |
| | III | T47D | 2.3 × | 2.6 × | 8.8 × | 15/20 | 0/5 |
| | | | 10¹² | 10⁷ | 10⁴ | | |
| | IV | OVCAR | 2.5 × | 5.2 × | 4.8 × | 2/20 | 0/1 |
| | | | 10¹² | 10⁷ | 10⁴ | | |
| RAN2 | I | T47D | 3.0 × | 2.3 × | 1.3 × | 3/20 | |
| | | | 10¹² | 10⁷ | 10⁵ | | |
| | II | OVCAR | 2.4 × | 3.2 × | 7.5 × | 1/20 | |
| | | | 10¹² | 10⁶ | 10⁵ | | |
| | III | T47D | 2.5 × | 1.2 × | 2.1 × | 19/20 | 0/5 |
| | | | 10¹² | 10⁸ | 10⁴ | | |
| | IV | OVCAR | 2.1 × | 1.0 × | 2.0 × | 6/20 | 0/2 |
| | | | 10¹² | 10⁸ | 10⁴ | | |
| SPIKE | I | T47D | 3.2 × | 1.4 × | 2.3 × | 3/20 | 0/1 |
| | | | 10¹² | 10⁷ | 10⁴ | | |
| | II | OVCAR | 1.0 × | 1.6 × | 6.3 × | 5/20 | |
| | | | 10¹² | 10⁷ | 10⁴ | | |
| | III | T47D | 1.4 × | 1.8 × | 7.8 × | 20/20 | 0/7 |
| | | | 10¹² | 10⁹ | 10² | | |
| | IV | OVCAR | 1.7 × | 3.3 × | 5.2 × | 20/20 | 0/5 |
| | | | 10¹² | 10⁸ | 10³ | | |
| HSPOT | I | T47D | 3.0 × | 2.2 × | 1.4 × | 0/20 | |
| | | | 10¹² | 10⁸ | 10⁴ | | |
| | II | OVCAR | 1.2 × | 5.2 × | 2.3 × | 1/20 | |
| | | | 10¹² | 10⁶ | 10⁵ | | |
| | III | T47D | 1.5 × | 1.9 × | 7.9 × | 18/20 | 1/4 |
| | | | 10¹² | 10⁸ | 10³ | | |
| | IV | OVCAR | 1.2 × | 2.7 × | 4.4 × | 15/20 | 2/6 |
| | | | 10¹² | 10⁷ | 10⁴ | | |

Despite some variability, the input-output (I/O) ratio of the phage titers did not really increase over the course of four cell selections. Yet an increase was seen in the frequency of clones binding to the MUC1 peptide, as well as the appearance of non-wild type clones in all selected libraries (Table 8). Upon sequencing it was revealed that all of the clones from the RAN libraries were derived from the SPIKE library, most likely due to cross-contamination between libraries. This suggests that in the RAN libraries, there are not many high affinity variants of PH1 present.

Analysis of Representative Clones for Sequence and Affinity in BIAcore and FACS

Clones from the many different selection rounds were screened initially in BIAcore for improvement of binding towards the MUC1 peptide. From a large screening effort, in which a few hundred clones were screened from the various selection approaches, the best clones were identified for further characterization. An overview of the characterized PH1 CDR3 variants from all rounds of selection is given in Table 9.

TABLE 9
Characterization of a large panel of PH1 variants

| | relative | | | |
|---|---|---|---|---|
| | ELISA | | | |
| Clone | signal | FR3-CDR3 region | Freq. in sel. | SEQ ID NUMBER |
| WT-PH1 | +(+) | AK HTGGGVWDPIDY | | 97-110 of SEQ ID NO:3 |
| 5C8 | +++ | -- ---R------G- | | SEQ ID NO:29 |
| 7D1 | +++ | -- ----------KH | | SEQ ID NO:30 |
| 7F9 | +++ | -- ----------G- | | SEQ ID NO:31 |
| 7F3 | +++ | -I ----------K- | | SEQ ID NO:32 |
| 10C10 | +++ | -- ---V------K- | | SEQ ID NO:73 |
| 11C1 | +++ | -- ---E------K- | | SEQ ID NO:74 |
| 3A7 | +++ | -- -----------K | | SEQ ID NO:75 |
| 6B6 | +++ | -- ----------G- | | SEQ ID NO:76 |
| 10B3 | +++ | -- ----------G- | 3x | SEQ ID NO:76 |
| 11G9 | +++ | -- ----------G- | | SEQ ID NO:76 |
| 10A8 | +++ | -R-----------G- | | SEQ ID NO:77 |
| 6F4 | +++ | S- ----------G- | | SEQ ID NO:78 |
| 6B3 | +++ | -- ----------GH | | SEQ ID NO:79 |
| 10F9 | +++ | -- -------N--GH | | SEQ ID NO:80 |
| 3B9 | +++ | -- ---------LG- | | SEQ ID NO:81 |
| 3B10 | +++ | -- ---------L-N | | SEQ ID NO:52 |
| 3D8 | +++ | -- ----------N- | 2x | SEQ ID NO:53 |
| 6B9 | +++ | -- ----------N- | | SEQ ID NO:53 |
| 3D10 | +++ | -- ----------N- | 2x | SEQ ID NO:83 |
| 6F3 | +++ | -- ----------N- | | SEQ ID NO:83 |
| 7DB | +++ | -- ----------N- | | SEQ ID NO:53 |
| 10B6 | +++ | -- ----------N- | 8x | SEQ ID NO:83 |
| 11E3 | +++ | -- ----------N- | 3x | SEQ ID NO:83 |
| 11B9 | +++ | -R ----------N- | | SEQ ID NO:84 |
| 6A9 | +++ | -- ---S------N- | | SEQ ID NO:85 |
| 6C8 | +++ | -- ----------ND | | SEQ ID NO:86 |
| 11F7 | +++ | -- ---V-----MN- | | SEQ ID NO:87 |
| 11F9 | +++ | T- ----------N- | | SEQ ID NO:88 |
| 3E2 | +++ | -- ----------A- | | SEQ ID NO:89 |
| 6B5 | +++ | -- ----------A- | | SEQ ID NO:89 |
| 7B5 | +++ | -- ----------A- | | SEQ ID NO:89 |
| 8F5 | +++ | -- ----------A- | | SEQ ID NO:89 |
| 10E1 | +++ | -- ----------A- | | SEQ ID NO:89 |
| 3F4 | +++ | -- ----------AN | | SEQ ID NO:90 |
| 3H1 | +++ | -- ---------FA- | | SEQ ID NO:91 |
| 11D4 | +++ | -- ---------MAS | | SEQ ID NO:92 |
| 3H2 | +++ | -- ---------M-- | | SEQ ID NO:93 |
| 6C10 | +++ | -- ----------H- | | SEQ ID NO:94 |
| 11F2 | +++ | -I ---A------R- | | SEQ ID NO:95 |
| 11F4 | +++ | -- ----------SS | | SEQ ID NO:96 |
| 3G1 | ++ | -- -----------D | | SEQ ID NO:97 |
| 6C5 | ++ | V- ----------V- | | SEQ ID NO:98 |
| 6E4 | +(+) | -- ---------V-- | | SEQ ID NO:99 |
| 10F3 | +(+) | -- ----------VP | | SEQ ID NO:100 |
| 10C3 | +(+) | V- ----------A- | | SEQ ID NO:102 |
| 10A9 | +(+) | -- ----------HN | | SEQ ID NO:102 |
| 10F8 | +(+) | -- ---------MH- | | SEQ ID NO:103 |
| 10E10 | +(+) | -- -------N---- | | SEQ ID NO:104 |
| 5A6 | + | V- ------------ | | SEQ ID NO:105 |
| 3B8 | + | -- ---A-------- | | SEQ ID NO:106 |
| 3D7 | + | -- ---A-------- | | SEQ ID NO:106 |
| 3D1 | + | -Q ----------G- | | SEQ ID NO:107 |
| 3F3 | + | -- ---R-------- | | SEQ ID NO:108 |
| 3F7 | + | -- ----------Y- | | SEQ ID NO:109 |

The first number of the clone name in Table 9 indicates its origin: 3-4, directly selected on MUC1 antigen; 5-6-7-8, selected with PH1 competition; 10-11, cell selected. The clones were ranked according to their relative ELISA signal (as soluble Fab fragments). Sequencing of the clones revealed that most of the observed variability in the clones with the strongest signals targeted a few residues in the CDR3 only, and were nearly exclusively found as derived from the SPIKE library. Indeed, the residues most frequently mutated in these clones, were not targeted in the RAN libraries. Within these clones, there is a strong conservation visible of most of the core region of the CDR3, the regions randomized in the RAN libraries, with a lot of mutations visible in the FR3 region and the J-encoded region of the CDR3. In many clones residues K98 (in SEQ ID NO:3) and/or D109 (in SEQ ID NO:3) are frequently mutated, thereby most likely disrupting the putative salt bridge between these charged amino acids. Not all substitutions are allowed; for example mutations to valine or alanine may disrupt this salt bridge, but do not confer a higher affinity. There was some variability at position 1 of the VH , caused by use of oligonucleotide #206, which allows either glutamate (E) or glutamine (Q) to be incorporated; often both variants were found carrying the same mutations in the FR3-CDR3 region, but this never affected the affinity (data not shown). There was little bias in the diversity of the clones selected with the three different procedures (direct selection, selection using competition with a MUC1 peptide antigen, or selection on cells). Indeed, variants with a single substitution at position 109, to glycine (G) or to asparagine (N) are frequently selected in all selection procedures (Table 9).

From the HSPOT library two variants were tested: clone 7G8 (from the competition selection) and clone 10G9 (from the cell selection). Both had a mutation at position 31 in the CDR1 of the VH, more specifically S31N and S31R for 7G8 and 10G9, respectively. The ELISA signal for these clones did not reach the signals seen for most CDR3 variants, and the clones were not further analyzed.

The CDR3 variants were extensively tested in BIAcore for the kinetics of the MUC1 peptide interaction. The off-rate of the wild-type clone could not be determined because it was too fast for analysis; however, based on its Kd (1.4 micromolar), an improvement of the off-rate of over 10-fold should result in a detectable change in off-rate in this assay. Using this off-rate screening with Fabs in the periplasmic extract of E. coli cultures, the clones in Table 9 as well as many more were screened for improvement in off-rate. The best clone, 5C8, was derived from the competition selection (Table 7), and showed a clear increase in off-rate. To get accurate measurements, a Kd assay on the BIAcore was used with the MUC1 15-mer (there was no difference in the kinetics of interaction when the MUC1 60-mer was used, data not shown). Clone 5C8 showed a 3.5-fold increase of the Kd over wild type PH1 Fab antibody (see, Table 10). Some other candidate clones, including 3D6, from the direct selection, and three other clones from a more stringent competition selection clone (i.e., 7D1, 7F3 and 7F9) were extensively investigated using BIAcore and/or flow cytometry analysis. The BIAcore data in Table 10 highlight the data of both the Kd values for peptide binding in BIAcore as well as the sequence differences between these clones; of all variants, clone 5C8 appears to have the best affinity. A single mutation D109G, as in clone 7F9, yields less than a 2-fold improvement, but an additional G102R mutation, as in clone 5C8, brings the affinity gain to 3.5-fold.

A flow cytometry experiment was carried out to determine the relative affinities of these Fabs versus wild type on cellular MUC1, although the data are not directly comparable (data not shown). The relative ranking of the three clones from highest to lowest affinity (i.e., 7D1>7F3>7F9) appear to have stayed the same, but the positioning of both the wild type clone PH1 and best BIAcore mutant 5C8 appearef to be different from what was expected on the basis of the BIAcore affinity. This apparent discrepancy between the binding affinity for the MUC1 peptide and for the cell surface MUC1 is most likely caused by the effect of partial glycosylation of the antibody epitopes of MUC1 glycoprotein on cells, which may effect binding in a different manner depending on the antibody fine specificity and interaction with the MUC1 antigen.

Although it appeared preferable to select and screen antibody affinity variants on cellular MUC1, rather than on a peptide source of the antigen, the selection of the PH1-based antibody libraries on cells did not yield any higher affinity variants than 5C8. Most of the MUC1 peptide binding selected variants, as well as selected clones without detectable peptide binding, harbored sequence variations that were found in clones selected on MUC1 peptide (Table 9 and data not shown).

### Example 3

Production and Characterization of a Recombinant, Human MUC1-Specific Immunoglobulin Molecule PH1-IgG

As described above in Example 1, the MUC1-specific PH1 Fab antibody was selected from a very large phage library displaying 3.7×10¹⁰ Fab antibody molecules. The PH1 Fab antibody has a Kd of 1.4 micromolar (μM) in BIAcore analysis using the MUC1 60-mer peptide antigen. This example demonstrates a method to increase the apparent affinity of a Fab antibody of the invention for cellular MUC1 expressed on cancer cells and tissues by changing the format from the single (monovalent) antigen binding site of the Fab antibody to the two (divalent) binding site format of an immunoglobulin molecule, such as IgG. As described below, a completely human, recombinant PH1-IgG1 antibody molecule was made by cloning the V_{H} and V_{L} genes of PH1 into a mammalian expression vector system (Persic et al., Gene, 187: 9-18 (1997)). The recombinant expression vectors were then cotransfected into mammalian CHO-K1 cells for expression.

Cloning the V_{H} and V_{L} of PH1 Fab Antibody into a Human IgG Molecule

The heavy and the light chains (i.e., V_{H} and V_{L} ) of the PH1 human Fab antibody were recloned into the mammalian VHexpress and VKexpress expression vectors, respectively, for producing a fully human gamma-1/kappa IgG1 antibody (Persic et al., Gene, 187: 9-18 (1997)). The V_{H} fragment of PH1 was amplified by PCR using specific oligonucleotides VH1C Back eukaryotic (5′-GGA CTA GTC CTG GAG TGC GCG CAC TCC CAG GTC CAG CTG GTG CAG TCT GGG GGA GGC TTG GTA CAG-3′ (SEQ ID NO:110)) and M13 commercial sequencing primer (Amersham Pharmacia, Upsala, Sweden), and introduced into the VH express vector as BssHII/BstEII fragment. An ApaL1/Pac1 fragment of PH1 V_{L} was generated by PCR using specific oligonucleotides VKexpress-MUC-for (5′-GCG CTC GCA TTT GCC TGT TAA TTA AGT TAG ATC TAT TCT ACT CAC GTT TGA TAT CCA CTT TGG TCC CAG GGC C-3′ (SEQ ID NO:111)) and MUC1-VL-Back-APA (5′-CCA GTG CAC TCC GAA ATT GTG CTG ACT CAG TCT CC-3′ (SEQ ID NO:112)), and inserted into VKexpress. Transfections of CHO-K1 (ATCC, Manassas, Va.) cells were carried out using a non-liposomal transfection reagent FuGene 6 (Roche, Brussels, Belgium) according to manufacturer's instructions.

Screening of Cell Culture Supernatants in ELISA

Supernatants of clones growing on medium containing selection markers were tested in ELISA for antibody binding to MUC1 and to determine V_{H}/V_{L} production levels. For MUC1 binding tests, the method of Henderickx et al. (Henderickx et al., Cancer Res., 58: 4324-4332 (1998)) was adapted for use in this study. Incubation volumes were 100 μl. MUC1 peptide antigen (i.e., 0.5 μg/ml biotinylated MUC1 60-mer) was immobilized indirectly on a flexible microtiter plate via streptavidin bound to biotinylated BSA, which was coated on the wells of the microtiter plate. Immobilizing MUC1 60-mer was done overnight at 4° C. After three washes with PBS, plates were blocked by incubating 30 minutes at room temperature (RT) with 2% (w/v) skimmed milk powder (Marvel) in PBS. Plates were washed two times with PBS-0.1% Tween 20 and once with PBS, and supernatants were then incubated for 1.5 hours at RT while shaking (diluted 1:4 in 2% (w/v) Marvel/PBS). Subsequently, plates were washed five times with PBS-0.1% Tween 20 and once with PBS. Bound IgG was detected with rabbit anti-human HRP-conjugated IgG (1:6000 diluted in 2% Marvel/PBS). Following the last incubation, staining was performed with tetramethylbenzidine (TMB) and H₂O₂ as substrate and stopped by adding 0.5 volume of 2N H₂SO₄. The optical density was measured at 450 nanometers (nm).

Production of Human IgG

To determine the amount of human PH1-IgG1 produced, a plate was coated for 1 hour at 37° C. with 0.25 μg/ml rabbit anti-human Vκ immunoglobulin in PBS. After three washes with PBS, plates were blocked during 30 minutes at RT with 2% (w/v) semi-skim milk powder (Marvel) in PBS. Plates were washed two times with PBS-0.1% Tween 20 and once with PBS. Supematants were then incubated for 1.5 hour at RT while shaking (diluted 1:4 in 2% (w/v) Marvel/PBS). A 2-fold dilution series of human IgG (huIgG) was used as a standard, starting with a concentration of 500 ng/ml. Subsequently, plates were washed five times with PBS-0.1% Tween 20 and once with PBS. Bound IgG was detected with rabbit anti-human IgG HRP (1 μg/ml in 2% Marvel/PBS). Following the last incubation, staining was performed with tetramethylbenzidine and H₂O₂ as substrate and stopped by adding 0.5 volume of 2N H₂SO₄; the optical density was measured at 450 nm.

Production and Purification of the PH1-IgG1 from Culture Media of CHO-K1 Clone 7F Cells

Approximately 3×10⁸ transfected CHO-K 1 cells (clone 7F) were cultured in T175 triple-layer flasks in a humidified incubator at 37° C. for 3 weeks. The culture medium contained 0.5% fetal calf serum (FCS) and was exchanged once each week. From each harvest, about 1 liter of culture supernatant was obtained. Anti-MUC1 antibody was purified with Protein A. Briefly, 1 liter of culture supernatant was loaded onto a 5 ml HiTrap Protein A column (Amersham/Pharmacia) at a flow rate of 5 ml/minute. The column was extensively washed with PBS. Bound MUC1 antibody was eluted with 12.5 mM citric acid and neutralized with 0.5 M HEPES (pH 9). Protein containing fractions were combined, dialyzed against PBS (16 hours, 4° C.) and sterile filtered. Purified anti-MUC1 antibody was analyzed by SDS-PAGE and silver staining, human IgG specific ELISA, and a BCA micro protein assay (Pierce). PH1-IgG (100-200 ng), purified with Protein A, was separated on a 10% SDS-PAGE gel (Laemmli et al., J. Mol. Biol., 47: 69-85 (1970)) under reducing conditions, and protein bands were visualized by silver staining. For Western blots, purified PH1-IgG was separated on a 10% SDS-PAGE gel under reducing conditions and transferred onto nitrocellulose. PH1-IgG heavy chain and light chain were simultaneously detected with a HRP-conjugated polyclonal antibody against human IgG and an HRP-conjugated monoclonal antibody against human kappa chain, respectively. Production amount was measured in a human IgG ELISA described above.

Surface Plasmon Resonance

The selected PH1-IgG1 and the Fab PH1 antibodies were evaluated for their binding characteristics by surface plasmon resonance on a BIAcore 2000 apparatus (BIAcore AB, Uppsala, Sweden). A biotin chip was coated with a MUC1 15-mer, containing the minimal PH1 epitope, PAP (Ac-PDTRPAPGSTAPPAL-NH₂ (SEQ ID NO:40) (see Example 2, above), 50 RU and 320 RU) and 60-mer (NH₂-(VTSAPDTRPAPGSTAPPAHG)₃-COOH (SEQ ID NO:8) (von Mensdorff-Pouilly et al., Tumor Biol., 19: 186-195 (1998), 50 RU) in HBS-EP buffer (Pharmacia) a surface, blocked with biotin (15 RU), was used as a negative control. The Fab PH1 and PH1-IgG1 were injected in HBS-EP buffer. To minimize rebinding of the antigen binding molecules, a speed of 30 μl/sec was used. Affinity calculation was performed with computer programs provided by BIAcore (BIAEvaluation-version3, BIACore AB). Fitting was accepted when Chi² was lowest, on the two channels with a non-saturated amount (50 RU) of MUC1 peptide bound. The affinity for the PH1 Fab antibody was calculated according to a 1:1 Langmuir stoichiometry at steady state (Chi²: 50.6). Because of the two binding places on the PH1-IgG1, the avidity was calculated as an apparent avidity constant using 1:1 Langmuir determination with mass transfer limitation (Chi²: 42).

Flow Cytometric Analysis

Cellular MUC1 binding was tested in flow cytometry, with PH1-IgG purified as before and with the murine HMFG1 antibody (Autogen Bioclear, Wilthshire, UK). About 500,000 cells were used in each experiment: after trypsinisation, cells were washed one time in RPMI 10% FCS, 0.01% NaN₃ (incubation buffer). To confirm the specificity, the same amount (100 μg/ml) of specific antibodies or non-binding human antibody and with or without 100 μg/ml MUC1 60-mer for 1 hour at room temperature were used. Then the samples were added to the cells and left for 1 hour at room temperature. Cells were spun down by centrifugation for 3 minutes at 611×g. Between incubations, cells were washed twice with incubation buffer. Anti-human IgG1 antibody was added to the cells and incubated for 1 hour at room temperature. Then rabbit anti-mouse-FITC was added to all tubes, and the tubes were incubated for 30 minutes. Detection of bound antibodies was performed by means of flow cytometry on a FACSCalibur (Becton Dickinson, Oxnard), and results analyzed with the CELLQuest program (Becton Dickinson, Oxnard).

Cell lines used in the study were the mouse fibroblast cell line 3T3, the MUC1 transfected cell line 3T3-MUC1 (ETA) (Acres et al., J. Immunother., 14: 136-143 (1993)), the breast carcinoma lines T47D and MCF-7, the ovarian carcinoma line OVCAR-3, the colon cancer cell line LS174T, the colon cell line CaCo2, and the T cell line Jurkat (non-transfected cell lines were provided by ATCC).

Biotinylation and FITC-labeling of PH1-IgG

PH1-IgG1 in 50 mM NaHCO₃, pH 8.5, at a concentration of 250 μg/ml was treated with sulfo-NHS-LC-biotin (Pierce, New York, N.Y.) for 1 hour at RT under gentle agitation. 4 μg of biotin ester was used for 100 μg of the antibody. The reaction was stopped by treatment with Tris/HCl, pH 7.5, at a final concentration of 50 mM, for 30 minutes. To separate the biotinylated antibody from free biotin, the reaction mixture was dialyzed against PBS. Biotinylation of PH1-IgG was verified by flow cytometry analysis by binding of the antibody to the MUC1 positive OVCAR3 cells and ETA cells compared to the MUC1 negative 3T3 cells.

FITC-labeling was performed according to the manufacturer with 200 μg PH-IgG1 in 200 μl reaction mixture using a FITC protein labeling kit (Molecular Probes, Leiden, Netherlands). Labeling was checked on MUC1 positive and negative cell lines in flow cytometry analysis (ETA, OVCAR-3, 3T3).

Immunohistochemistry

A variety of formalin-fixed normal and tumor tissues were tested for the binding pattern of the PH1-IgG1. Tissues were chosen with a preference for diagnosed adenocarcinoma. HMFG-1 was used as a control for a limited number of tumor tissues. The biotinylated PH1-IgG1 antibody was used. Slices (5 μm) of paraffin-embedded tissues were de-paraffinized, rehydrated, hydrogen peroxide treated (0.3% H₂O₂ in PBS), and preincubated with PBS, 15% FCS, 5% human serum (HS) for 20 minutes. Antibodies were diluted to a concentration of 17 μg/ml in PBS, 10% HS and incubated for 1 hour at room temperature. For PH1-IgG 1, slides were then incubated with an avidin-biotin-complex (ABC, Dako, Glostrup, Denmark) for 30 minutes. For HMFG1, slides were first incubated with biotinylated sheep-anti-mouse (RAMPO, Dako) in PBS, 0.1% Tween 20, 1% BSA for 30 minutes and then with the avidin-biotin-complex. For each tissue, a negative control with non-binding human IgG1 was used. Between antibody incubation, slides were washed three times for 5 minutes in PBS. Staining was carried out using diaminobenzidin (DAB) and H₂O₂. The peroxidase reaction was stopped with water, and slides were counter-stained with hematoxylin. The epithelial tissues were evaluated for their binding reactivity (sporadic: <10%, focal: 10%<f<80%, diffuse: >80%) and their localization in the cell (a: apical, polar, c: cytoplasmic, depolarized, m: abundantly expression on the cell membrane). To study glycosylation sensitivity, a normal breast tissue section was pre-treated with periodic acid in acetate buffer 0.05 M, pH 5 for 30 min at room temperature in the dark as described by (Cao et al., Tumour Biol., 19 Suppl. 1: 88-99 (1998)).

Evaluation of Internalization Using a Confocal Microscope

Antibody was FITC labeled according to the manufacturer's instructions (see above). The FITC-labeled antibody bound in flow cytometry to the ETA and OVCAR-3 cells and not to the MUC1 negative 3T3 cell line (data not shown). For internalization studies, the human tumor cell line OVCAR-3 and the MUC1 transfected mouse fibroblast 3T3 cell line, ETA, were used. As negative control, the colon cell line CaCo2 was used. FITC-labeled antibody was added to the cells (10 μg/10⁶ cells at a concentration of 100 μg/ml) for an incubation period of 1 hour on ice. The cells were washed and put on ice to check whether the antibody stayed bound to the membrane or placed at 37° C. to study internalization. At each time point (1, 3, 6 hours and overnight), cells were checked on a confocal microscope for membrane binding and internalization. Fc binding was checked by competition with human IgG1. Staining patterns (membranous or intracellular) were evaluated with a confocal microscope (Asciophat, Zeiss, Atto Instrument, Rockville, Md.).

Cloning of PH1-IgG1 into a Mammalian Expression Vector and Selection of Transfectants

In this study, the human PH1 Fab antibody (Example 1) directed to MUC1 was recloned as a fully human gamma-1/kappa immunoglobulin antibody into the mammalian VHexpress and VKexpress expression vectors. DNA containing a sequence encoding the PH1-V_{H} was cloned into VHexpress, and DNA containing a sequence encoding the PH1-V_{L} fragment was inserted into the expression cassette of VKexpress. Co-transfection of VHexpress and VKexpress recombinant vectors into CHO-K1 cells was carried out using the non-liposomal transfection reagent FuGene 6. At 48 hour after transfection, limiting dilutions were performed into medium containing 700 μg/ml G418. Cells were plated in 96-well plates at 10, 100 and 1000 cells per well. On the 100 cell/well plate, 36 out of 96 wells showed cell growth after 5 days in culture. Supernatants of grown, positive wells were assayed for presence of human gamma-immunoglobulins and binding to MUC1-peptide in ELISA. Of these, 13 were positive for binding to MUC1, with a range of detected human IgG between 5 and 77 ng/ml. Clone 7F (75 ng/ml) was chosen for further study. To guarantee clonality, an additional round of subcloning was carried out (data not shown).

Production and Purification of the PH1-IgG1

The MUC1-specific PH1-IgG1 antibody was purified from 0.5% FCS containing culture media as described above. Under these conditions, no co-purification of bovine IgG appeared, and more than 90% pure PH1-IgG1 protein was obtained as evidenced on silver stained SDS-PAGE. The results of a human IgG1 specific ELISA and a BCA total protein detection assay were in good agreement (data not shown). From 1 liter of culture media, about 0.5 mg PH1-IgG were purified, approximately corresponding to an expression level of 0.3 pg per cell, derived from approximately 3×10⁸ cells within 1 week.

BIAcore Analysis

The affinity of the antibody was determined using BIAcore. Affinities of the Fab PH1 were calculated to be an average of 1.4 μM for binding to the 15-mer and 60-mer MUC1 peptide antigen coated surfaces. Mean avidity of PH1-IgG1 (8.7 nM) was calculated with the BIACore software from binding curves on low density surfaces being 8.3 nM (15-mer) and 9.06 nM (60-mer). The binding affinity of the PH1-IgG1 antibody was found to be over 100 times stronger than with the parent Fab PH1 antibody molecule.

Comparative Flow Cytometric Analysis

Since differences in the fine-specificity of MUC1 antibodies can lead to differences in the panel of tissues and tumors recognized, the PH1-IgG1 antibody was compared with a frequently used murine antibody, HMFG1. PH1-IgG1 recognizes the PAP epitope as determined by epitope fingerprinting of the PH1 Fab (Example 1, above; Henderickx et al., Cancer Res., 58: 43224-4332 (1998)), while HMFG1 recognizes the PDTR (amino acids 9-12 of SEQ ID NO:7) epitope. The two antibodies were tested on different tumor cell lines in flow cytometry. Both antibodies bound with the same binding pattern to most of the cell lines, except for the ovarian carcinoma cell line OVCAR-3, which apparently exposes more of the PH1-IgG1 epitope than the HMFG1 epitope. Both antibodies bind a small subpopulation of the LS 174T colon tumor cell line and of the T cell line Jurkat, which can be inhibited by MUC1 60-mer. No binding to the CaCo2 colon cell line was observed. Binding of MUC1 to cells could be competed off with MUC1 peptide, although the competition appeared not to be quantitative.

This study indicated that there is a difference in the spread and/or density of the various MUC1 epitopes or a differential accessibility of these epitopes due to residual glycosylation. To understand the abundance of the PH1-IgG1 MUC1-epitope, it was necessary to carry out immunohistochemical analysis on a large set of tissues and tumors (see, below).

Immunohistochemical Analysis of PH1-IgG

An immunohistochemical analysis was carried out on a large set of tissues and tumors (see, Table 10 below). The general degree of MUC1 localization (“staining”) in tumor cells was (from most to least staining) depolarized cytoplasmic (c)>abundant membranous staining of the whole cell (m)>polarized apical (a), while in normal tissues the localization pattern was a>c>m (see, Table 10). In Faddition, staining reactivity was higher in tumor tissues than in normal tissues (data not shown).

TABLE 10
Immunohistological staining of normal and tumor epithelial
tissues with PH1-IgG1.

| | Normal tissues | | Tumor tissues* | | | |
|---|---|---|---|---|---|---|
| | Reac- | Local- | Reac- | Local- | | |
| Tissue | tivity | ization | tivity | ization | Freq. | Remarks |
| Bladder | — | — | | | 2/2 | |
| | | | s | a | 1/4 | Transitional |
| | | | f | a | 1/4 | Urothelial |
| | | | f | a, c | 1/4 | |
| | | | d | c, m | 1/4 | |
| Colon | — | — | | | 3/3 | |
| | | | — | — | 1/2 | Squamous |
| | | | f | a, c | 1/2 | Mucinous |
| Endometrium | | | | | | |
| | f | a | | | 2/6 | |
| | f | c | | | 1/6 | |
| | d, f | a, c | | | 2/6 | |
| | | | f | a, c, m | 1/1 | |
| Epididymis | f | a | | | 3/3 | |
| Kidney | | | | | 5/5 | |
| glomeruli | — | — | | | | |
| prox. tub. | — | — | | | | |
| dist. tub. | f | a | | | | |
| coll. ducts | d | a | | | | |
| Liver | — | — | | | 3/3 | |
| bile duct | s | a | | | | |
| | | | — | — | 1/1 | Hepato- |
| | | | | | | cellular |
| Lung | — | — | | | 6/6 | |
| | | | f | c, m | 2/5 | 1 squamous |
| | | | f | a, m | 1/5 | 1 squamous |
| | | | f | a, c, m | 1/5 | |
| | | | f | a | 1/5 | |
| Mamma | f | a | | | 4/5 | |
| | — | — | | | 1/5 | |
| | | | f | a, c, m | 3/7 | |
| | | | d | a, c, m | 2/7 | |
| | | | d | a, m | 1/7 | |
| | | | f | a | 1/7 | Papiloma |
| Ovarian | f | a | | | 2/2 | |
| | | | d | c, m | 2/8 | |
| | | | f | a, c, m | 1/8 | |
| | | | d | a, c, m | 1/8 | |
| | | | f | c | 2/8 | |
| | | | d | c | 1/8 | |
| | | | f | a | 1/8 | Sereus |
| Pancreas | | | | | | |
| acini | d | a | | | 5/5 | |
| exocrine gl. | d | a(c) | | | | |
| isl. Langerhans | — | — | | | | |
| | | | f | a, c | 1/2 | |
| | | | d | a, m, c | 1/2 | |
| Parathyroid | — | — | | | 3/3 | |
| | | | f | a, c | 1/2 | |
| | | | f | c | 1/2 | |
| Prostate | — | — | | | 5/6 | |
| | s | a | | | 1/6 | |
| | | | — | — | 1/3 | |
| | | | d | a, c | 1/3 | |
| | | | d | c, m | 1/3 | |
| Salivary gland | | | | | | |
| ducti | d-f | a-c | | | 2/2 | |
| acini | —-f | —-a | | | | |
| Skin | | | | | | |
| sebaceous gl. | d | m | | | | |
| sweat gland | f | a | | | | |
| hair follicle | — | — | | | | |
| Testes | — | — | | | 3/3 | |
| | | | — | — | 1/1 | |
| Tuba | f | a | | | 2/2 | |
| Thyroid | — | — | | | 2/2 | |
| | | | — | — | 1/1 | |
| Vas deferens | f | a | | | 1/1 | |

*Tumors are adenocarcinoma, except when stated differently.
Abbreviations:
s: sporadic staining (<10%), f: focal staining (10 < s < 80%), d: diffuse staining (>80%); a: polarized apical, c: depolarized cytoplasmic, m: abundantly present on whole cell membrane

A summary of the study of localization of MUC1 using the PH1-IgG antibody in various tissues follows.

Normal bladder was negative in cases tested. Tumor tissues of the bladder had different staining patterns in which both adenocarcinoma tissues had a depolarized staining pattern. Colon cancer, normal tissues, and squamous carcinoma were negative. A mucinous tumor tested in this study had depolarized cytoplasmic staining. In endometrium, some normal tissues showed a depolarized localization. In normal kidney, the staining pattern was always the same with no staining in glomeruli and proximal tubes, focal apical staining in distal tubes and diffuse, apical staining in collecting ducts. In contrast, with lung tissues, normal lung (negative), and adenocarcinoma of the lung was intensively MUC1 positive in a depolarized fashion. In most tumors, an extensive staining of whole cell membranes was found.

Not all tumor cells, per tissue, reacted with the antibody (i.e., focal staining observed). In breast and ovarian adenocarcinoma tissues, there was a differential staining between normal and adenocarcinoma, being polarized in normal and cytoplasmic with membranous staining in adenocarcinoma (6/6 for breast, 4/7 for ovarian adenocarcinoma). Intensity of staining was less in normal tissue than in tumor tissue. The reactivity was diffuse to focal in tumor tissues and focal in normal tissues.

Pancreas adenocarcinoma had a cytoplasmic staining pattern. Normal acini expressed MUC1 apically, and exocrine glands showed a polar staining or cytoplasmic staining. In normal tissues of the endometrium and sebaceous gland of the skin, a depolarized staining pattern for MUC1 was observed. Periodate-treated normal breast epithelium was stained slightly more intensively than the non-treated tissue, indicating that, as expected, de-glycosylation exposes the epitope of PH1.

Taken together, the above study showed that a differential expression of MUC1 was found between normal tissue and tumor in bladder, lung, breast, ovary, pancreas, parathyroid, and prostate tissue. Apical staining was found in normal tissues as well as in tumor tissues, depolarized cellular (cytoplasmic) staining was most frequently detected in tumors, and aberrant staining of the whole cell membrane was only found in tumors with the exception of the sebaceous glands of the skin.

A comparison with the murine HMFG1 antibody for a limited amount of tissues is shown in Table 11. Normal tissues were stained mainly focally apical, except for an endometrium tissue that showed cytoplasmic staining with PH1-IgG1. In tumors, small differences in immunoreactivity were seen which can be confirmed with a larger panel of tissues.

TABLE 11
Comparison in immunohistochemistry between human PH1-IgG1 and
the mouse HMFG1 antibodies

| | HMFG1 | | PH1 | | |
|---|---|---|---|---|---|
| | Distri- | Local- | Distri- | Local- | |
| | bution | ization | bution | ization | Freq. |
| Bladder (N)* | — | — | — | — | 1 |
| Breast (N) | f | a | f | a | 3 |
| Breast (T) | d | a | f | a, c | 1 |
| Breast papiloma | — | — | f | a | 1 |
| Breast (T) | f | m | d | a, c, m | 1 |
| Breast (T)* | d | m, c | f | a | 1 |
| Liver | — | — | — | — | 1 |
| Parathyroid (T) | f | a | d | a, m | 1 |
| Tuba (N)* | f | a | f | a | 1 |
| Endometrium (N) | f | a | f | c | 1 |
| Ovarium (T) | f | c, m | f | c, m | 1 |
| Ovarian (T) | d | a | f | a, c, m | 1 |
| Ovarian (N) | — | — | — | — | 1 |
| Ovarian (T) | d | a | f | a, c | 1 |
| Ovarian (T) | f | a | f | a | 1 |
| Ovarian (T) | d | c, m | d | c, m | 2 |

*T: Tumor tissue, N: Normal tissue
Abbreviations:
a: polarized apical, c: depolarized cytoplasmic, m: abundantly present on whole cell membrane

Evaluation of Internalization of PH1-IgG1, Using Confocal Microscope

To analyze the extent with which PH1-IgG1 after binding would be internalized, an internalization study using FITC-labeled antibody was carried out. The FITC-labeled antibody bound in FACS analysis to the OVCAR-3 and ETA cell lines, and not to the negative 3T3 cell line (data not shown). After 1 hour of incubation on ice with the human antibody PH1-IgG1 , membranous binding was observed on the MUC1 expressing OVCAR-3 and ETA cell lines. As in flow cytometry, the intensity of staining was more pronounced for the ETA cell line as compared with the OVCAR-3 cell line. No auto-fluorescence was observed, and no fluorescence was visible on the CaCo2 negative control cell line. At 37° C., the internalization of the PH1-IgG1-FITC became visible for both the ETA cells and the OVCAR-3 cells. After 1 hour, more than 50% of OVCAR-3 cells had internalized the antibody in vesicles, while the ETA cells had mainly membrane bound antibody. After 3 hours of incubation, more than 80% of the FITC-labeled antibody was internalized by the OVCAR-3 cells: vesicles were visible but also cells with a low level of intracellular fluorescence were visible. After 6 hours, all OVCAR-3 cells had internalized the antibody, and most cells had lost the vesicle internalization pattern and exhibited a low cytoplasmic fluorescence only. At either 3 or 6 hours, OVCAR-3 cells kept on ice had the antibody still bound to the membrane only. The ETA cells had internalized less than 3% of the antibody after 3 hours, but after overnight incubation, the surviving cells had internalized the antibody and no membrane bound antibody was left. In contrast, cells kept overnight on ice showed membranous staining.

Analysis

This study characterized a recombinant, anti-MUC1 antibody formed by recloning the V_{H} and V_{L} regions of the MUC1-specific Fab antibody PH1 into a two-vector, mammalian cell expression system to produce a new, fully human, whole IgG1, which has significantly enhanced affinity for MUC1 compared to the PH1 Fab parent molecule. The somewhat low yield, when compared to the production of other antibodies in CHO-K1 cells (for a review, see Trill et al., Curr. Opin. Biotechnol., 6: 553-560 (1995)), is probably caused by differential expression of the light and the heavy chain and the yet not undertaken optimization of culture conditions. The amount produced was, nevertheless, sufficient for the small-scale production of the antibody for the various laboratory tests described above. For immunotherapy, such characterization is important in order to determine whether a particular antibody will fit a particular therapy or vice versa, especially since all MUC1 antibodies do not behave the same (Cao et al., Tumour Biol., 19 Suppl. 1: 88-99 (1998); Pietersz et al., Cancer Immunol. Immuother., 44: 323-328 (1997)).

First, the affinity of the antibody is a major determining factor in establishing how fast it will bind to a tumor cell and how quickly it will release itself from the antigen-bearing tumor cell. In this study, the avidity of the newly generated antibody was compared with the affinity of the original Fab in BIAcore. Avidities for the PH1 Fab and PH1-IgG were 1.4 μM and 8.7 nM respectively, indicating a 100-fold increase for the whole human antibody (PH1-IgG1). This avidity change is solely due to the change from one to two binding sites, since binding on the 60-mer and 15-mer channel are comparable. Comparison between diabodies obtained from single chain antibodies (scFvs) to ErB2 with different affinities showed that the magnitude of the decrease in the apparent dissociation rate constant (Kd) for the bivalent molecule was inversely proportional to the affinities of the scFvs (Nielsen et al., Cancer Res., 60: 6434-6440 (2000)). The PH1 Fab antibody has a relatively low affinity, and the increase of apparent affinity for the corresponding PH1-IgG molecule is very high, confirming the above observation from Nielsen.

In flow cytometric analysis, PH1-IgG1 was compared with HMFG1, which is reported to recognize a different, glycosylation sensitive, MUC1 epitope (Cao et al., 1998; Burchell et al., Epithelial Cell Biol., 2; 155-162 (1993)). The binding pattern on tumor cell lines did not differ significantly between both antibodies, except for the OVCAR-3 cell line, which was stained less by HMFG1, probably due to the different epitope recognition. On colon cancer cell lines, both antibodies hardly showed any binding. Colon cancer cells can be highly glycosylated, and glycosylation sensitive antibodies rarely stain this glycosylated colon mucin (Sikut et al., Tumour Biol., 19 Suppl. 1: 122-126 (1998); Blockzjil et al., Tumour Biol., 19 Suppl. 1: 46-56 (1998)). This suggests that the antibody PH1-IgG1 recognizes MUC1 in an underglycosylated form, which is expected to be tumor-associated. The antibody C595, binding the RPAP epitope, reacts in FACS analysis to OVCAR-3 and MCF-7 cells with the same pattern as HMFG1 (Reddish et al., Tumour Biol., 19 Suppl. 1: 57-66 (1998)) and consequently also with PH1-IgG1. The antibodies did bind well to the T47D breast cancer cell line known to express different glycoforms of MUC1 (Hanisch et al., Eur. J. Biochem., 236: 318-327 (1996)). The usage of periodate on a normal breast tissue intensified the apical staining confirming the glycosylation sensitivity of this antibody as for many antibodies recognizing an epitope on the protein core of MUC1 (Cao et al., 1998).

Immunohistochemical staining revealed a differential staining between tumor tissues and normal tissues, being apical or absent in normal tissues and depolarized in tumor tissues as described for glycosylation sensitive antibodies (Zotter et al., Cancer Rev., 11-12: 56-101 (1988); Cao et al., 1998). In normal tissues of the ovary and breast, staining was often heterogeneous (f) and not as intense as in tumor. In breast and ovarian tumors, staining was diffuse or heterogeneous, and intense membrane staining was found in 6/6 breast and 4/7 of the ovarian adenocarcinoma. Thus, MUC1 is ubiquitously present on cell membranes. In bladder and lung, differences between tumor and normal tissues are highest. In normal tissues, tested the PH1-IgG1 epitope is not present. This is in contrast with the findings of weakly to focally positive reactivity with monoclonal antibodies recognizing the PDTR (amino acids 9-12 of SEQ ID NO:7) region of MUC1 core protein in normal lung and bladder tissues (Zotter et al., 1988; Walsh et al., Br. J. Urol., 73: 256-262 (1994)). In tumor tissues, heterogeneous staining was observed with mostly focal reactivity in both lung and bladder. In all 20 adenocarcinomas tissues, the PH1-IgG1 epitope is expressed in a non-polar fashion.

Although the staining pattern of the PH1 epitope is different with staining patterns of other glycosylation sensitive antibodies (Zotter et al., 1988), in some cases the PH1-IgG1 meets or even exceeds expectations. The immunohistochemical staining patterns support, as in flow cytometry, that the antibody PH1-IgG1 indeed binds to the underglycosylated tumor-associated MUC1 that is abundantly expressed in a depolarized fashion in adenocarcinoma. Such antibodies, recognizing an epitope of the MUC1 tandem repeat, are described for murine (derived) antibodies and are successfully used in targeting studies in humans (von Hof et al., Cancer Res., 565: 5179-5185 (1996); Biassoni et al., Br. J. Cancer, 77: 131-138 (1998); Kramer et al., Clin. Cancer Res., 4: 1679-1688 (1998)).

Although the peptide epitope is PAP (SEQ ID NO: ), PH1-IgG1 binds specifically and preferentially to underglycosylated MUC. Spencer et al. (Cancer Lett., 100: 11-15 (1996)) investigated the influence of glycosylation on antibody binding with their antibody recognizing the minimal epitope RPAP (amino acids 12-15 of SEQ ID NO:7) and concluded that this antibody in positively influence by glycosylation. This in contrast with an antibody recognizing the PDTR (amino acids 9-12 of SEQ ID NO:7) motif. This could explain the different fine-specificity of the PH1-IgG. The Fab antibody PH1 was selected by phage display technology, by two rounds of selection on ETA cells and 3 rounds of selection on a MUC1 60-mer (see, Example 1). Possibly, by the way the antibody was selected, it favors binding to an underglycosylated epitope PAP of the tandem repeat.

The data indicate that the PH1-IgG antibody would be particularly useful as a targeting tool in bladder, lung, mammary, and ovarian cancer where the PH1-IgG1 epitope is, in most cases, present on the tumor cells in a depolarized fashion (c, m in Table 11). Because of the possible heterogeneous (focal) expression, the PH1-IgG antibody could be used in an immunotherapy that has a bystander effect on surrounding tumor cells, e.g., radio-immunotherapy, a combination of radio-immunotherapy and immunotoxins (see, e.g., Wei et al., Clin. Cancer Res., 6: 631-642 (2000)), or in the use of fusion proteins that stimulate tumor infiltrating lymphocytes (see, e.g., Lode et al., Pharmacol. Therap., 80: 277-292 (1998)). The abundance of expression of the PH1-IgG1 epitope on the membranes of tumor cells is heterogeneously spread. Because of the high amount of MUC1 on the their membranes, these cells provide excellent targets for PH1-IgG. Again, supporting the use of PH1-IgG in a therapy with bystander effects.

Internalization studies demonstrated that the FITC-labeled antibody is internalized by both OVCAR-3 and ETA cells, although with a different rapidity. First, the internalization pattern was almost exclusively in vesicles. Later, the vesicle structure was less abundant and faint staining was found in the cytoplasm. This could be due to the breakdown of the antibody, leaving free FITC in the cytoplasm, or due to a modification of the FITC, itself, and loss of its fluorescence. After 1 hour at 37° C., more than half of the OVCAR-3 cells exhibited fluorescent vesicles, meaning that the antibody rapidly internalized into vesicles. It has been described that the MUC1 antigen recycles 0.9% of surface fraction/minute (Litvino et al., J. Biol. Chem., 268: 221364-21371 (1993)). This study confirms the observation (data not shown) that at 1 hour more than 50% of the cells have internalized the antibody.

Internalization of MUC1 antibodies is not always the same and may depend on the epitope. Pietersz et al. (1997) compared two antibodies for their internalization rate, the antibody specific for MUC1 epitope RPAP (amino acids 12-15 of SEQ ID NO:7) (CTMO1) internalized much better than the antibody specific for the PDTR (amino acids 9-12 of SEQ ID NO:7) epitope. The PH1-IgG antibody, when assayed with the peptide epitope PAP, appears to have a similar internalization rate. The MUC1 transfected 3T3 cell line, ETA, internalized the FITC-labeled antibody much slower. At first sight this could be due to the fact that mouse cells normally do not express human MUC1 and that the internalization machinery is not effective for this xenogenic protein. Some transfected cell lines may internalize better than others (see, e.g., Pietersz et al., 1997). Because of the internalization, the PH1-IgG antibody can be used in a variety of therapies and combination, such as for immunotherapy with pro-drugs, drugs, for gene therapy (for a review of such various therapies, see Syrigos et al., Hybridoma, 18: 219-224 (1999)), and for radio-immunotherapy, where it may not always be necessary that the radiolabel is internalized.

In conclusion, the human antibody PH1-IgG1 was shown to recognize tumor-associated MUC1 on adenocarcinoma. Its affinity is high enough to bind to tumor cells and because the FITC-labeled antibody can be internalized by recycled MUC1, it is a candidate molecule for therapeutic and diagnostic tumor targeting applications, especially in lung, bladder, ovarian, and breast adenocarcinoma.

All documents and publications cited above are incorporated herein by reference.

Other variations and embodiments of the invention described herein will now be apparent to those of ordinary skill in the art without departing from the scope of the invention or the spirit of the claims below.

1. An isolated MUC1-specific binding member comprising an antigen binding domain, wherein the antigen binding domain comprises a region comprising the amino acid sequence of the formula:
X₁ X₂ His Thr Gly X₃ Gly Val Trp X₄ Pro X₅ X₆ X₇ (SEQ ID NO:28),
wherein
X₁ is Ala, Ser, Thr, or Val;
X₂ is Lys, Ile Arg, or Gln;
X₃ is Gly, Arg, Val, Glu, Ser, or Ala;
X₄ is Asp or Asn;
X₅ is Ile, Leu, Met, Phe, or Val;
X₆ is Asp, Gly, Lys, Asn, Ala, His, Arg, Ser, Val, or Tyr; and
X₇ is Tyr, His, Lys, Asn, Asp, Ser, Pro.

2. The MUC1-specific binding member according to claim 1, wherein the variable region comprises the amino acid sequence selected from the group consisting of:

| | |
|---|---|
| Ala Lys His Thr Gly Gly Gly Val Trp Asp Pro Ile Asp Tyr | (amino acids 97-110 of SEQ ID NO:3); |
| Ala Lys His Thr Gly Arg Gly Val Trp Asp Pro Ile Gly Tyr | (SEQ ID NO:29); |
| Ala Lys His Thr Gly Gly Gly Val Trp Asp Pro Ile Lys His | (SEQ ID NO:30); |
| Ala Lys His Thr Gly Gly Gly Val Trp Asp Pro Ile Gly Tyr | (SEQ ID NO:3 1); and |
| Ala Ile His Thr Gly Gly Gly Val Trp Asp Pro Ile Lys Tyr | (SEQ ID NO:32). |

3. An isolated MUC1-specific binding member comprising an antigen binding domain comprising an antibody V_{L} region comprising the amino acid sequence of SEQ ID NO:1, or portion thereof, and an antibody V_{H} region comprising the amino acid sequence of SEQ ID NO:3, or portion thereof.

4. A MUC1-specific binding member comprising an antigen binding domain, wherein the antigen binding domain comprises a CDR of an antibody V_{L} or V_{H} region, wherein said CDR has an amino acid sequence selected from the group consisting of amino acids 24 to 39 of SEQ ID NO:1, amino acids 55 to 61 of SEQ ID NO:1, amino acids 94 to 102 of SEQ ID NO:1, amino acids 31 to 35 of SEQ ID NO:3, amino acids 50 to 66 of SEQ ID NO:3, amino acids 99 to 110 of SEQ ID NO:3, conservatively substituted sequences of any of the preceding sequences, and combinations thereof.

5. The MUC1-specific binding member according to any of claim 1, 2, 3, or 4, wherein said MUC1-specific binding member is a fusion protein.

6. The MUC1-specific binding member according to any one of claim 1, 2, 3, or 4, further comprising a detectable label or tag.

7. The MUC1-specific binding member according to claim 6, wherein the detectable label or tag is selected from the group consisting of epitope tags, fluorescent labels, radioactive labels, heavy metals, anti-cancer drugs, toxins, and magnetic resonance imaging labels.

8. The MUC1-specific binding member according to any one of claim 1, 2, 3, or 4, wherein the MUC1-specific binding member is an antibody molecule selected from the group consisting of immunoglobulins, Fab antibodies, F(ab′)₂ antibodies, diabodies, scFv antibodies, double scFv, Fv molecules, dAb, immunocytokine molecules, and immunotoxin molecules.

9. The MUC1-specific immunocytokine according to claim 8, comprising the amino acid sequence of SEQ ID NO:5.

10. The MUC1-specific immunocytokine according to claim 9, further comprising a detectable label or tag.

11. The MUC1-specific binding member according to claim 10, wherein the detectable label or tag is selected from the group consisting of, epitope tags, fluorescent labels, radioactive labels, and magnetic resonance imaging labels.

12. The MUC1-specific immunoglobulin according to claim 8, comprising a light chain polypeptide comprising the amino acid sequence of SEQ ID NO:24 and a heavy chain polypeptide comprising the amino acid sequence of SEQ ID NO:26.

13. The MUC1-specific immunoglobulin according to claim 12, further comprising a detectable label or tag.

14. The MUC1-specific binding member according to claim 13, wherein the detectable label or tag is selected from the group consisting of enzymes, epitope tags, fluorescent labels, radioactive labels, heavy metals, anti-cancer drugs, toxins, and magnetic resonance imaging labels.

15. A MUC1-specific binding member comprising an antibody antigen binding domain comprising a heavy chain variable region, or CDR thereof, from the DP47 germ line.

16. A MUC1-specific binding member comprising an antibody antigen binding domain comprising a light chain variable region, or a CDR thereof, from the DPK15 germ line.

17. A MUC1-specific binding member comprising an antibody antigen binding domain comprising a heavy chain variable region, or CDR thereof, from the DP47 germ line and a light chain variable region, or CDR thereof, from the DPK15 germ line.

18. A MUC1-specific binding member comprising an amino acid sequence that is about 70% or more homologous to any of the amino acid sequences of claim 1, 2, 3, or 4.

19. A MUC1-specific binding member comprising an amino acid sequence that is about 80% or more homologous to any of the amino acid sequences of claim 1, 2, 3, or 4.

20. A MUC1-specific binding member comprising an amino acid sequence that is about 90% or more homologous to any of the amino acid sequences of claim 1, 2, 3, or 4.

21. A MUC1-specific binding member comprising an amino acid sequence that is about 95% or more homologous to any of the amino acid sequences of claim 1, 2, 3, or 4.

22. A MUC1-specific binding member comprising an amino acid sequence that is about 97% or more homologous to any of the amino acid sequences of claim 1, 2, 3, or 4.

23. A MUC1-specific binding member comprising an amino acid sequence that is about 99% or more homologous to any of the amino acid sequences of claim 1, 2, 3, or 4.

24. A polypeptide molecule comprising an amino acid sequence that is about 70% or more homologous to an amino acid sequence selected from the group consisting of SEQ ID NO:1, amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQ ID NO:3, amino acids 50 to 66 of SEQ ID NO:3, amino acids 99 to 110 of SEQ ID NO:3, and SEQ ID NO:5.

25. A polypeptide molecule comprising an amino acid sequence that is about 80% or more homologous to an amino acid sequence selected from the group consisting of SEQ ID NO:1, amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQ ID NO:3, amino acids 50 to 66 of SEQ ID NO:3, amino acids 99 to 110 of SEQ ID NO:3, and SEQ ID NO:5.

26. A polypeptide molecule comprising an amino acid sequence that is about 90% or more homologous to an amino acid sequence selected from the group consisting of SEQ ID NO:1, amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQ ID NO:3, amino acids 50 to 66 of SEQ ID NO:3, amino acids 99 to 110 of SEQ ID NO:3, and SEQ ID NO:5.

27. A polypeptide molecule comprising an amino acid sequence that is about 95% or more homologous to an amino acid sequence selected from the group consisting of SEQ ID NO:1, amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQ ID NO:3, amino acids 50 to 66 of SEQ ID NO:3, amino acids 99 to 110 of SEQ ID NO:3, and SEQ ID NO:5.

28. A polypeptide molecule comprising an amino acid sequence that is about 97% or more homologous to an amino acid sequence selected from the group consisting of SEQ ID NO:1, amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQ ID NO:3, amino acids 50 to 66 of SEQ ID NO:3, amino acids 99 to 110 of SEQ ID NO:3, and SEQ ID NO:5.

29. A polypeptide molecule comprising an amino acid sequence that is about 99% or more homologous to an amino acid sequence selected from the group consisting of SEQ ID NO:1, amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQ ID NO:3, amino acids 50 to 66 of SEQ ID NO:3, amino acids 99 to 110 of SEQ ID NO:3, and SEQ ID NO:5.

30. An isolated polynucleotide molecule comprising a nucleotide sequence encoding an amino acid sequence selected from the group consisting of SEQ ID NO:1, amino acids 24 to 39 of SEQ ID NO:1, amino acids 55 to 61 of SEQ ID NO:1, amino acids 94 to 102 of SEQ ID NO:1, SEQ ID NO:3, amino acids 31 to 35 of SEQID NO:3, amino acids 50 to 66 of SEQID NO:3, amino acids 99 to 110 of SEQ ID NO:3, SEQ ID NO:5, and combinations thereof.

31. An isolated polynucleotide molecule comprising a nucleotide sequence encoding an amino acid sequence selected from the group consisting of SEQ ID NO:24 and SEQ ID NO:26.

32. An isolated polynucleotide molecule encoding a V_{L} region comprising a nucleotide sequence of SEQ ID NO:2 or degenerate sequences thereof.

33. An isolated polynucleotide molecule encoding a V_{L} region comprising a nucleotide sequence which is about 70% or more homologous to the sequence of SEQ ID NO:2.

34. An isolated polynucleotide molecule encoding a V_{L} region comprising a nucleotide sequence which is about 80% or more homologous to the sequence of SEQ ID NO:2.

35. An isolated polynucleotide molecule encoding a V_{L} region comprising a nucleotide sequence which is about 90% or more homologous to the sequence of SEQ ID NO:2.

36. An isolated polynucleotide molecule encoding a V_{L} region comprising a nucleotide which is about 95% or more homologous to the sequence of SEQ ID NO:2.

37. An isolated polynucleotide molecule encoding a V_{L} region comprising a nucleotide sequence which is about 97% or more homologous to the sequence of SEQ ID NO:2.

38. An isolated polynucleotide molecule encoding a V_{L} region comprising a nucleotide sequence which is about 99% or more homologous to the sequence of SEQ ID NO:2.

39. An isolated polynucleotide molecule encoding a V_{H} region comprising a nucleotide sequence of SEQ ID NO:4, or degenerate sequences thereof.

40. An isolated polynucleotide molecule encoding a V_{H} region comprising a nucleotide which is about 70% homologous to the sequence of SEQ ID NO:4.

41. An isolated polynucleotide molecule encoding a V_{H} region comprising a nucleotide which is about 80% homologous to the sequence of SEQ ID NO:4.

42. An isolated polynucleotide molecule encoding a V_{H} region comprising a nucleotide which is about 90% homologous to the sequence of SEQ ID NO:4.

43. An isolated polynucleotide molecule encoding a V_{H} region comprising a nucleotide which is about 95% homologous to the sequence of SEQ ID NO:4.

44. An isolated polynucleotide molecule encoding a V_{H} region comprising a nucleotide which is about 97% homologous to the sequence of SEQ ID NO:4.

45. An isolated polynucleotide molecule encoding a V_{H} region comprising a nucleotide which is about 99% homologous to the sequence of SEQ ID NO:4.

46. An isolated polynucleotide molecule encoding a CDR of an antibody variable region icomprising a nucleotide sequence selected from the group consisting of nucleotides 70 to 117 of SEQ ID NO:2, nucleotides 163 to 183 of SEQ ID NO:2, nucleotides 280 to 306 of SEQ ID NO:2, nucleotides 91 to 105 of SEQ ID NO:4, nucleotides 148 to 198 of SEQ ID NO:4, nucleotides 295 to 330 of SEQ ID NO:4, degenerate sequences of any of the preceding CDR coding sequences, and combinations thereof.

47. An isolated polynucleotide molecule comprising a nucleotide sequence that is about 60% or more homologous to a nucleotide sequence selected from the group consisting of SEQ ID NO:2, SEQ ID NO:4, SEQ ID NO:6, nucleotides 70 to 117 of SEQ ID NO:2, nucleotides 163 to 183 of SEQ ID NO:2, nucleotides 280 to 306 of SEQ ID NO:2, nucleotides 91 to 105 of SEQ ID NO:4, nucleotides 148 to 198 of SEQ ID NO:4, and nucleotides 295 to 330 of SEQ ID NO:4.

48. An isolated polynucleotide molecule comprising a nucleotide sequence that is about 70% or more homologous to a nucleotide sequence selected from the group consisting of SEQ ID NO:2, SEQ ID NO:4, SEQ ID NO:6, nucleotides 70 to 117 of SEQ ID NO:2, nucleotides 163 to 183 of SEQ ID NO:2, nucleotides 280 to 306 of SEQ ID NO:2, nucleotides 91 to 105 of SEQ ID NO:4, nucleotides 148 to 198 of SEQ ID NO:4, and nucleotides 295 to 330 of SEQ ID NO:4.

49. An isolated polynucleotide molecule comprising a nucleotide sequence that is about 80% or more homologous to a nucleotide sequence selected from the group consisting of SEQ ID NO:2, SEQ ID NO:4, SEQ ID NO:6, nucleotides 70 to 117 of SEQ ID NO:2, nucleotides 163 to 183 of SEQ ID NO:2, nucleotides 280 to 306 of SEQ ID NO:2, nucleotides 91 to 105 of SEQ ID NO:4, nucleotides 148 to 198 of SEQ ID NO:4, and nucleotides 295 to 330 of SEQ ID NO:4.

50. An isolated polynucleotide molecule comprising a nucleotide sequence that is about 90% or more homologous to a nucleotide sequence selected from the group consisting of SEQ ID NO:2, SEQ ID NO:4, SEQ ID NO:6, nucleotides 70 to 117 of SEQ ID NO:2, nucleotides 163 to 183 of SEQ ID NO:2, nucleotides 280 to 306 of SEQ ID NO:2, nucleotides 91 to 105 of SEQ ID NO:4, nucleotides 148 to 198 of SEQ ID NO:4, and nucleotides 295 to 330 of SEQ ID NO:4.

51. An isolated polynucleotide molecule comprising a nucleotide sequence that is about 95% or more homologous to a nucleotide sequence selected from the group consisting of SEQ ID NO:2, SEQ ID NO:4, SEQ ID NO:6, nucleotides 70 to 117 of SEQ ID NO:2, nucleotides 163 to 183 of SEQ ID NO:2, nucleotides 280 to 306 of SEQ ID NO:2, nucleotides 91 to 105 of SEQ ID NO:4, nucleotides 148 to 198 of SEQ ID NO:4, and nucleotides 295 to 330 of SEQ ID NO:4.

52. An isolated polynucleotide molecule comprising a nucleotide sequence that is about 97% or more homologous to a nucleotide sequence selected from the group consisting of SEQ ID NO:2, SEQ ID NO:4, SEQ ID NO:6, nucleotides 70 to 117 of SEQ ID NO:2, nucleotides 163 to 183 of SEQ ID NO:2, nucleotides 280 to 306 of SEQ ID NO:2, nucleotides 91 to 105 of SEQ ID NO:4, nucleotides 148 to 198 of SEQ ID NO:4, and nucleotides 295 to 330 of SEQ ID NO:4.

53. An isolated polynucleotide molecule encoding a MUC1-specific binding member comprising the nucleotide sequence of SEQ ID NO:6.

54. The isolated polynucleotide molecule according to any one of claims 30-53, wherein the polynucleotide molecule is a molecule selected from the group consisting of linear polynucleotide molecules, plasmids, phagemids, bacteriophage vectors, yeast display vectors, and eukaryotic viral vectors.

55. A method of diagnosing cancer in an individual comprising:
providing a biological sample from the individual;
contacting the biological sample from the individual with a MUC1-specific binding member according to any one of claims 1-23, conservatively substituted versions of any of the preceding sequences, and combinations thereof; and
detecting binding of said MUC1-specific binding member to MUC1 in the biological sample of the individual.

56. The method of diagnosing cancer in an individual according to claim 55, wherein the cancer is adenocarcinoma.

57. The method of diagnosing cancer in an individual according to claim 55, wherein the biological sample from the individual is selected from the group consisting of cells, blood, lymph, urine, mammary tissue, ovary tissue, lung tissue, bladder tissue, and combinations thereof.

58. The method of diagnosing cancer in an individual according to claim 55, wherein the binding of said MUC1-specific binding member to MUC1 is detected by a detection means selected from the group consisting of enzyme-linked immunosorbent assay, magnetic resonance imaging, scintillation counting, and X-ray film.

59. A method of treating cancer in an individual comprising:
administering to the individual in need of treatment thereof a MUC1-specific binding member according to any one of claims 1-23, conservatively substituted versions of any of the preceding sequences, and combinations thereof.

60. The method of treating cancer in an individual according to claim 59, wherein the cancer is adenocarcinoma.

61. The method of treating cancer in an individual according to claim 59, further comprising administering a cytokine to the individual before, contemporaneously with, or after administering the MUC1-specific binding member.

62. The method of treating cancer in an individual according to claim 59, wherein the cancer is present in tissue of the breast, ovary, lung, or bladder of the individual.

63. An ex vivo method of treating cancer in an individual comprising:
obtaining a body fluid containing MUC1 and/or MUC1-expressing cancer cells from an individual;
contacting the body fluid with an immobilized MUC1-specific binding member according to any one of claims 1-23, conservatively substituted versions of any of the preceding sequences, and combinations thereof;
collecting the body fluid not bound to the immobilized MUC1-specific binding member; and
returning the collected body fluid not bound to the immobilized MUC1-specific binding member to the individual.

64. The ex vivo method of treating cancer according to claim 63, further comprising the step of adding one or more therapeutic agents to the body fluid prior to returning the fluid to the individual.

65. The ex vivo method of treating cancer according to claim 63, wherein the body fluid is selected from the group consisting of bone marrow, blood, and peripheral blood stem cells.

66. The ex vivo method of treating cancer according to claim 63, wherein the cancer is adenocarcinoma.

67. The ex vivo method of treating cancer according to claim 63, wherein the anti-cancer reagent is a MUC1-specific binding member.

68. A method of making a MUC1-specific binding member comprising:
preparing an expression vector comprising a polynucleotide sequence according to any of claims 30-54, conservatively substituted versions of any of the preceding sequences, and combinations thereof;
inserting said expression vector into a host cell; and
culturing said host cell under conditions in which the MUC1-specific binding member is expressed from the expression vector.

69. The method of making a MUC1-specific binding member according to claim 68, wherein the MUC1-specific binding member is selected from the group consisting of an immunoglobulin, a Fab antibody, F(ab′)₂ antibody, a diabody, a scFv, a double scFv, a dAb, a Fv, an immunotoxin, and an immunocytokine.


SEQUENCE LISTING (112 sequences)

SEQ ID NO: 1 - protein, 113 aa, artificial sequence
  light chain variable region of the MUC1- specific binding domain of PH1 Fab antibody
EIVLTQSPLSLPVTPGEPASISCRSSQSLLHSNGYTYLDWYLQKPGQSPQLLIYSGSHRASGVPDRFSGSVSGTDFTLRISRVEAEDVGVYYCMQGLQSPFTFGPGTKVDIKR

SEQ ID NO: 2 - DNA, 339 bp, artificial sequence
  nucleotide sequence coding for amino acid sequence of SEQ ID NO1
gaaattgtgctgactcagtctccactctccctgcccgtcacccctggagagccggcctccatctcctgcaggtctagtcagagcctcctgcatagtaatggatacacctatttggattggtacctgcagaagccagggcagtctccacagctcctgatctattcgggttctcatcgggcctccggggtccctgacaggttcagtggcagtgtatcaggcacagattttacactgagaatcagcagagtggaggctgaggatgttggagtttattactgcatgcagggtctacagagtccattcactttcggccctgggaccaaagtggatatcaaacga

SEQ ID NO: 3 - protein, 121 aa, artificial sequence
  heavy chain variable region of the MUC1- specific binding domain of the PH1 Fab antibody
QVQLVQSGGGLVQPGGSLRLSCAASGFTFRSNAMGWVRQAPGKGLEWVSGISGSGGSTYYADSVKGRFTISRDNSKNTLYLQMNSLRAEDTAVYYCAKHTGGGVWDPIDYWGQGTLVTVSS

SEQ ID NO: 4 - DNA, 363 bp, artificial sequence
  nucleotide sequence coding for amino acid sequence of SEQ ID NO3
caggtccagctggtgcagtctgggggaggcttggtacagcctggggggtccctgagactctcctgtgcagcctctggattcacgtttagaagtaacgccatgggctgggtccgccaggctccagggaaggggctggagtgggtctcaggtattagtggtagtggtggcagcacatactacgcagactccgtgaagggccggttcaccatctccagagacaattccaagaacacgctgtatctgcaaatgaacagcctgagagccgaggacacggccgtatattattgtgcgaaacataccggggggggcgtttgggaccccattgactactggggccagggaaccctggtcaccgtctcaagc

SEQ ID NO: 5 - protein, 381 aa, artificial sequence [sequence omitted]

SEQ ID NO: 6 - DNA, 1143 bp, artificial sequence
  nucleotide sequence coding for amino acid sequence of SEQ ID NO5
caggtccagctggtgcagtctgggggaggcttggtacagcctggggggtccctgagactctcctgtgcagcctctggattcacgtttagaagtaacgccatgggctgggtccgccaggctccagggaaggggctggagtgggtctcaggtattagtggtagtggtggcagcacatactacgcagactccgtgaagggccggttcaccatctccagagacaattccaagaacacgctgtatctgcaaatgaacagcctgagagccgaggacacggccgtatattattgtgcgaaacataccggggggggcgtttgggaccccattgactactggggccagggaaccctggtcaccgtctcaagcggaggcggtgcacttgaaattgtgctgactcagtctccactctccctgcccgtcacccctggagagccggcctccatctcctgcaggtctagtcagagcctcctgcatagtaatggatacacctatttggattggtacctgcagaagccagggcagtctccacagctcctgatctattcgggttctcatcgggcctccggggtccctgacaggttcagtggcagtgtatcaggcacagattttacactgagaatcagcagagtggaggctgaggatgttggagtttattactgcatgcagggtctacagagtccattcactttcggccctgggaccaaagtggatatcaaacgagggggtggatcaggcggcggggccctagcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 7 - protein, 20 aa, artificial sequence
  peptide of MUC1 protein
PAHGVTSAPDTRPAPGSTAP

SEQ ID NO: 8 - protein, 20 aa, artificial sequence
  peptide of MUC1 protein
VTSAPDTRPAPGSTAPPAHG

SEQ ID NO: 9 - DNA, 24 bp, artificial sequence
  CH1FOR primer
gtccttgaccaggcagcccagggc

SEQ ID NO: 10 - DNA, 23 bp, artificial sequence
  pUC-reverse primer
agcggataacaatttcacacagg

SEQ ID NO: 11 - DNA, 44 bp, artificial sequence
  VL backward primer
accgcctccaccagtgcacttgaaattgtgctgactcagtctcc

SEQ ID NO: 12 - DNA, 51 bp, artificial sequence
  VL forward primer
accgcctccaccgggcgcgccttattaacactctcccctgttgaagctctt

SEQ ID NO: 13 - DNA, 61 bp, artificial sequence
  VL backward primer for light chain variable region of the PH1 Fab antibody with additional linker and restriction sites
gccgatcgctctggtcaccgtctcaagcggaggcggtgcacttgaaattgtgctgactcag

SEQ ID NO: 14 - DNA, 50 bp, artificial sequence
  VL forward primer for light chain variable region of PH1 Fab antibody with additional linker and restriction sites
gtctcgcgagcggccgccgattggatatccactttggtcccagggccgaa

SEQ ID NO: 15 - DNA, 27 bp, artificial sequence
  nucleotide sequence coding for a nine amino acid linker
gggggtggatcaggcggcggggcccta

SEQ ID NO: 16 - DNA, 69 bp, artificial sequence
  PH1-IL-2 backward primer
accaaagtggatatcaaacgagggggtggatcaggcggcggggccctagcacctacttcaagttctaca

SEQ ID NO: 17 - DNA, 49 bp, artificial sequence
  PH1-IL-2 forward primer
gtcccgcgtgcggccgcagtcagtgttgagatgatgctttgacaaaagg

SEQ ID NO: 18 - protein, 98 aa, artificial sequence
  heavy chain variable region from a DP47 germ line
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 19 - protein, 100 aa, artificial sequence
  light chain variable region from a DPK 15 germ line
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 20 - protein, 14 aa, artificial sequence
  myc tag peptide
EQKLISEEDLNGAA

SEQ ID NO: 21 - DNA, 42 bp, artificial sequence
  nucleotide sequence coding for amino acid sequence of SEQ ID NO20
gaacaaaaactcatctcagaagaggatctgaatggggccgca

SEQ ID NO: 22 - protein, 6 aa, artificial sequence
  hexahistidine peptide
HHHHHH

SEQ ID NO: 23 - DNA, 18 bp, artificial sequence
  nucleotide sequence coding for amino acid sequence of SEQ ID NO22
catcaccatcatcaccat

SEQ ID NO: 24 - protein, 220 aa, artificial sequence
  immunoglobulin kappa light chain of MUC1-specific PH1-IgG1
EIVLTQSPLSLPVTPGEPASISCRSSQSLLHSNGYTYLDWYLQKPGQSPQLLIYSGSHRASGVPDRFSGSVSGTDFTLRISRVEAEDVGVYYCMQGLQSPFTFGPGTKVDIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 663 bp, artificial sequence
  nucleotide sequence coding for amino acid sequence of SEQ ID NO24
gaaattgtgctgactcagtctccactctccctgcccgtcacccctggagagccggcctccatctcctgcaggtctagtcagagcctcctgcatagtaatggatacacctatttggattggtacctgcagaagccagggcagtctccacagctcctgatctattcgggttctcatcgggcctccggggtccctgacaggttcagtggcagtgtatcaggcacagattttacactgagaatcagcagagtggaggctgaggatgttggagtttattactgcatgcagggtctacagagtccattcactttcggccctgggaccaaagtggatatcaaacgaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcaccggtgacaaagagcttcaacaggggagagtgttag

SEQ ID NO: 26 - protein, 451 aa, artificial sequence
  immunoglobulin heavy chain of MUC1-specific PH1-IgG1
QVQLVQSGGGLVQPGGSLRLSCAASGFTFRSNAMGWVRQAPGKGLEWVSGISGSGGSTYYADSVKGRFTISRDNSKNTLYLQMNSLRAEDTAVYYCAKHTGGGVWDPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1356 bp, artificial sequence
  nucleotide sequence coding for amino acid sequence of SEQ ID NO26
caggtccagctggtgcagtctgggggaggcttggtacagcctggggggtccctgagactctcctgtgcagcctctggattcacgtttagaagtaacgccatgggctgggtccgccaggctccagggaaggggctggagtgggtctcaggtattagtggtagtggtggcagcacatactacgcagactccgtgaagggccggttcaccatctccagagacaattccaagaacacgctgtatctgcaaatgaacagcctgagagccgaggacacggccgtatattattgtgcgaaacataccggggggggcgtttgggaccccattgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccttaagtccgggaaaataa

SEQ ID NO: 28 - protein, 14 aa, artificial sequence
  variant (1)..(14): amino acid positions designated Xaa may be varied to form alternative regions of a MUC1-specific binding member as explained in the disclosure
XXHTGXGVWXPXXX

SEQ ID NO: 29 - protein, 14 aa, artificial sequence
  region of a MUC1-specific binding member
AKHTGRGVWDPIGY

SEQ ID NO: 30 - protein, 14 aa, artificial sequence
  region of a MUC1-specific binding member
AKHTGGGVWDPIKH

SEQ ID NO: 31 - protein, 14 aa, artificial sequence
  region of a MUC1-specific binding member
AKHTGGGVWDPIGY

SEQ ID NO: 32 - protein, 14 aa, artificial sequence
  amino acid sequence in a MUC1-specific binding member
AIHTGGGVWDPIKY

SEQ ID NO: 33 - DNA, 33 bp, artificial sequence
  misc_feature (1)..(33): n may be varied according to the disclosure to form mutagenic primer sequences
ggattcacgtttagannnaacgccatgggctgg

SEQ ID NO: 34 - DNA, 39 bp, artificial sequence
  misc_feature (1)..(39): n may be varied according to the disclosure to form mutagenic primer sequences
cacggagtctgcgtannntgtnnngccaccactaccact

SEQ ID NO: 35 - DNA, 90 bp, artificial sequence
  misc_feature (1)..(90): n may be varied according to the disclosure to form mutagenic primer sequences
ctatgagacggtgaccagggttccctggccccannnnnnnnnnnnnnnnnnnnnnnnnnnnnnnnnnnnnnnnnnacaataatatacggc

SEQ ID NO: 36 - DNA, 90 bp, artificial sequence
  misc_feature (1)..(90): n may be varied according to the disclosure to form mutagenic primer sequences
ctatgagacggtgaccagggttccctggccccagtagtcaatggggtcccaaacmnnmnnmnnmnnmnntttcgcacaataatatacggc

SEQ ID NO: 37 - DNA, 90 bp, artificial sequence
  misc_feature (1)..(90): n may be varied according to the disclosure to form mutagenic primer sequences
ctatgagacggtgaccagggttccctggccccagtagtcmnnmnnmnnmnnmnngccccccccggtatgtttcgcacaataatatacggc

SEQ ID NO: 38 - DNA, 24 bp, artificial sequence
  HuJH4-5-FOR primer
tgaggagacggtgaccagggttcc

SEQ ID NO: 39 - DNA, 56 bp, artificial sequence
  VH1c back Sfi primer
gtcctcgcaactgcggcccagccggccatggccsaggtccagctggtrcagtctgg

SEQ ID NO: 40 - protein, 15 aa, artificial sequence
  PAP peptide epitope of MUC1 bound by PH1 Fab antibody
PDTRPAPGSTAPPAL

SEQ ID NO: 41 - protein, 16 aa, artificial sequence
  mutant CDR3 region of an antibody heavy chain variable region
AKHNTSKVWDPIDYWG

SEQ ID NO: 42 - DNA, 48 bp, artificial sequence
  nucleotide sequence coding for a mutant CDR3 region
gcgaaacataatacgtctaaggtttgggaccccattgactactggggc

SEQ ID NO: 43 - protein, 16 aa, artificial sequence
  mutant CDR3 region of an antibody heavy chain variable region
AKSSTTTVWDPIDYWG

SEQ ID NO: 44 - DNA, 48 bp, artificial sequence
  nucleotide sequence coding for a mutant CDR3 region
gcgaaatctagtactacgacggtttgggaccccattgactactggggc

SEQ ID NO: 45 - protein, 16 aa, artificial sequence
  variant (1)..(16): Xaa, designated as “&”in the disclosure, indicates end of amino acid sequence because mutation in nucleotide sequence forms a translational stop codon
AKXPMANVWDPIDYWG

SEQ ID NO: 46 - DNA, 48 bp, artificial sequence
  nucleotide sequence coding for a mutant CDR3 region
gcgaaatagcctatggcgaatgtttgggaccccattgactactggggc

SEQ ID NO: 47 - protein, 16 aa, artificial sequence
  variant (1)..(16): Xaa, designated as “&”in the disclosure, indicates end of amino acid sequence because mutation in nucleotide sequence forms a translational stop codon
AKXHTKTVWDPIDYWG

SEQ ID NO: 48 - DNA, 48 bp, artificial sequence
  nucleotide sequence coding for a mutant CDR3 region
gcgaaatagcatacgaagacggtttgggaccccattgactactggggc

SEQ ID NO: 49 - protein, 16 aa, artificial sequence
  mutant CDR3 region of an antibody heavy chain variable region
AKITVSRVWDPIDYWG

SEQ ID NO: 50 - DNA, 48 bp, artificial sequence
  nucleotide sequence coding for a mutant CDR3 region
gcgaaaattactgttagtcgtgtttgggaccccattgactactggggc

SEQ ID NO: 51 - protein, 16 aa, artificial sequence
  mutant CDR3 region of an antibody heavy chain variable region
AKRYLYDVWDPIDYWG

SEQ ID NO: 52 - DNA, 48 bp, artificial sequence
  nucleotide sequence coding for a mutant CDR3 region
gcgaaacgttatctgtatgatgtttgggaccccattgactactggggc

SEQ ID NO: 53 - protein, 16 aa, artificial sequence
  mutant CDR3 region of an antibody heavy chain variable region
AKHTGGGTLQRLDYWG

SEQ ID NO: 54 - DNA, 48 bp, artificial sequence
  nucleotide sequence coding for a mutant CDR3 region
gcgaaacataccggggggggcactttgcagcggctggactactggggc

SEQ ID NO: 55 - protein, 16 aa, artificial sequence
  mutant CDR3 region of an antibody heavy chain variable region
AKHTGGGTQTPCDYWG

SEQ ID NO: 56 - DNA, 48 bp, artificial sequence
  nucleotide sequence coding for a mutant CDR3 region
gcgaaacataccggggggggcactcagactccgtgtgactactggggc

SEQ ID NO: 57 - protein, 16 aa, artificial sequence
  mutant CDR3 region of an antibody heavy chain variable region
AKHTGGGRRICHDYWG

SEQ ID NO: 58 - DNA, 48 bp, artificial sequence
  nucleotide sequence coding for a mutant CDR3 region
gcgaaacataccggggggggccgtcgtatttgtcatgactactggggc

SEQ ID NO: 59 - protein, 16 aa, artificial sequence
  variant (1)..(16): Xaa, designated as “&”in the disclosure, indicates end of amino acid sequence because mutation in nucleotide sequence forms a translational stop codon
AKHTGGGXRXXRDYWG

SEQ ID NO: 60 - DNA, 48 bp, artificial sequence
  nucleotide sequence coding for a mutant CDR3 region
gcgaaacataccggggggggctagcggtagtagcgggactactggggc

SEQ ID NO: 61 - protein, 16 aa, artificial sequence
  mutant CDR3 region of an antibody heavychain variable region
AKHTGGGQKLQLDYWG

SEQ ID NO: 62 - DNA, 48 bp, artificial sequence
  nucleotide sequence coding for a mutant CDR3 region
gcgaaacataccggggggggccagaagctgcagctggactactggggc

SEQ ID NO: 63 - protein, 16 aa, artificial sequence
  variant (1)..(16): Xaa, when designated as “&” in the disclosure, indicates end of amino acid sequence because mutation in nucleotide sequence forms a translational stop codon; or when designated as “s” in the disclosure, Xaa is serine
AXHTGGRGWDPIDYWG

SEQ ID NO: 64 - DNA, 48 bp, artificial sequence
  nucleotide sequence coding for a mutant CDR3 region
gcgtsacatacgggggggcgcggttgggaccccattgactactggggc

SEQ ID NO: 65 - protein, 16 aa, artificial sequence
  mutant CDR3 region of an antibody heavy chain variable region
ANQTGGGVWDPIDYWG

SEQ ID NO: 66 - DNA, 48 bp, artificial sequence
  nucleotide sequence coding for a mutant CDR3 region
gcgaaccagactggggggggcgtttgggaccccattgactactggggc

SEQ ID NO: 67 - protein, 16 aa, artificial sequence
  mutant CDR3 region of an antibody heavy chain variable region
ARHTGGGVWDPIYYWG

SEQ ID NO: 68 - DNA, 48 bp, artificial sequence
  nucleotide sequence coding for a mutant CDR3 region
gcgagacataccggtgggggcgtktgggatcccatatactactggggc

SEQ ID NO: 69 - protein, 16 aa, artificial sequence
  mutant CDR3 region of an antibody heavy chain variable region
AKPTGGGAWDPIDYWG

SEQ ID NO: 70 - DNA, 48 bp, artificial sequence
  nucleotide sequence coding for a mutant CDR3 region
gcgaaacctaccggggggggcgcttgggaccccattgactactggggc

SEQ ID NO: 71 - protein, 16 aa, artificial sequence
  mutant CDR3 region of an antibody heavy chain variable region
AKHTGVGVWHPIYYWG

SEQ ID NO: 72 - DNA, 48 bp, artificial sequence
  nucleotide sequence coding for a mutant CDR3 region
gcgaaacataccggggtgggcgtttggcaccccatctactactggggc

SEQ ID NO: 73 - protein, 14 aa, artificial sequence
  mutant CDR3 region of an antibody heavy chain variable region
AKHTGVGVWDPIKY

SEQ ID NO: 74 - protein, 14 aa, artificial sequence
  mutant CDR3 region of an antibody heavy chain variable region
AKHTGEGVWDPIKY

SEQ ID NO: 75 - protein, 14 aa, artificial sequence
  mutant CDR3 region of an antibody heavy chain variable region
AKHTGGGVWDPIDK

SEQ ID NO: 76 - protein, 14 aa, artificial sequence
  mutant CDR3 region of an antibody heavy chain variable region
AKHTGGGVWDPIGY

SEQ ID NO: 77 - protein, 14 aa, artificial sequence
  mutant CDR3 region of an antibody heavy chain variable region
ARHTGGGVWDPIGY

SEQ ID NO: 78 - protein, 14 aa, artificial sequence
  mutant CDR3 region of an antibody heavy chain variable region
SKHTGGGVWDPIGY

SEQ ID NO: 79 - protein, 14 aa, artificial sequence
  mutant CDR3 region of an antibody heavy chain variable region
AKHTGGGVWDPIGH

SEQ ID NO: 80 - protein, 14 aa, artificial sequence
  mutant CDR3 region of an antibody heavy chain variable region
AKHTGGGVWNPIGH

SEQ ID NO: 81 - protein, 14 aa, artificial sequence
  mutant CDR3 region of an antibody heavy chain variable region
AKHTGGGVWDPLGY

SEQ ID NO: 82 - protein, 14 aa, artificial sequence
  mutant CDR3 region of an antibody heavy chain variable region
AKHTGGGVWDPLDN

SEQ ID NO: 83 - protein, 14 aa, artificial sequence
  mutant CDR3 region of an antibody heavy chain variable region
AKHTGGGVWDPINY

SEQ ID NO: 84 - protein, 14 aa, artificial sequence
  mutant CDR3 region of an antibody heavy chain variable region
ARHTGGGVWDPINY

SEQ ID NO: 85 - protein, 14 aa, artificial sequence
  mutant CDR3 region of an antibody heavy chain variable region
AKHTGSGVWDPINY

SEQ ID NO: 86 - protein, 14 aa, artificial sequence
  mutant CDR3 region of an antibody heavy chain variable region
AKHTGGGVWDPIND

SEQ ID NO: 87 - protein, 14 aa, artificial sequence
  mutant CDR3 region of an antibody heavy chain variable region
AKHTGVGVWDPMNY

SEQ ID NO: 88 - protein, 14 aa, artificial sequence
  mutant CDR3 region of an antibody heavy chain variable region
TKHTGGGVWDPINY

SEQ ID NO: 89 - protein, 14 aa, artificial sequence
  mutant CDR3 region of an antibody heavy chain variable region
AKHTGGGVWDPIAY

SEQ ID NO: 90 - protein, 14 aa, artificial sequence
  mutant CDR3 region of an antibody heavy chain variable region
AKHTGGGVWDPIAN

SEQ ID NO: 91 - protein, 14 aa, artificial sequence
  mutant CDR3 region of an antibody heavy chain variable region
AKHTGGGVWDPFAY

SEQ ID NO: 92 - protein, 14 aa, artificial sequence
  mutant CDR3 region of an antibody heavy chain variable region
AKHTGGGVWDPMAS

SEQ ID NO: 93 - protein, 14 aa, artificial sequence
  mutant CDR3 region of an antibody heavy chain variable region
AKHTGGGVWDPMDY

SEQ ID NO: 94 - protein, 14 aa, artificial sequence
  mutant CDR3 region of an antibody heavy chain variable region
AKHTGGGVWDPIHY

SEQ ID NO: 95 - protein, 14 aa, artificial sequence
  mutant CDR3 region of an antibody heavy chain variable region
AIHTGAGVWDPIRY

SEQ ID NO: 96 - protein, 14 aa, artificial sequence
  mutant CDR3 region of an antibody heavy chain variable region
AKHTGGGVWDPISS

SEQ ID NO: 97 - protein, 14 aa, artificial sequence
  mutant CDR3 region of an antibody heavy chain variable region
AKHTGGGVWDPIDD

SEQ ID NO: 98 - protein, 14 aa, artificial sequence
  mutant CDR3 region of an antibody heavy chain variable region
VKHTGGGVWDPIVY

SEQ ID NO: 99 - protein, 14 aa, artificial sequence
  mutant CDR3 region of an antibody heavy chain variable region
AKHTGGGVWDPVDY

SEQ ID NO: 100 - protein, 14 aa, artificial sequence
  mutant CDR3 region of an antibody heavy chain variable region
AKHTGGGVWDPIVP

SEQ ID NO: 101 - protein, 14 aa, artificial sequence
  mutant CDR3 region of an antibody heavy chain variable region
VKHTGGGVWDPIAY

SEQ ID NO: 102 - protein, 14 aa, artificial sequence
  mutant CDR3 region of an antibody heavy chain variable region
AKHTGGGVWDPIHN

SEQ ID NO: 103 - protein, 14 aa, artificial sequence
  mutant CDR3 region of an antibody heavy chain variable region
AKHTGGGVWDPMHY

SEQ ID NO: 104 - protein, 14 aa, artificial sequence
  mutant CDR3 region of an antibody heavy chain variable region
AKHTGGGVWNPIDY

SEQ ID NO: 105 - protein, 14 aa, artificial sequence
  mutant CDR3 region of an antibody heavy chain variable region
VKHTGGGVWDPIDY

SEQ ID NO: 106 - protein, 14 aa, artificial sequence
  mutant CDR3 region of an antibody heavy chain variable region
AKHTGAGVWDPIDY

SEQ ID NO: 107 - protein, 14 aa, artificial sequence
  mutant CDR3 region of an antibody heavy chain variable region
AQHTGGGVWDPIGY

SEQ ID NO: 108 - protein, 14 aa, artificial sequence
  mutant CDR3 region of an antibody heavy chain variable region
AKHTGRGVWDPIDY

SEQ ID NO: 109 - protein, 14 aa, artificial sequence
  mutant CDR3 region of an antibody heavy chain variable region
AKHTGGGVWDPIYY

SEQ ID NO: 110 - DNA, 66 bp, artificial sequence
  VH1C Back eukaryotic primer
ggactagtcctggagtgcgcgcactcccaggtccagctggtgcagtctgggggaggcttggtacag

SEQ ID NO: 111 - DNA, 73 bp, artificial sequence
  VKexpress-MUC-for primer
gcgctcgcatttgcctgttaattaagttagatctattctactcacgtttgatatccactttggtcccagggcc

SEQ ID NO: 112 - DNA, 35 bp, artificial sequence
  MUC1-VL-Back-APA primer
ccagtgcactccgaaattgtgctgactcagtctcc